(19) United States
(12) Patent Application Publication
(10) Pub. No.: US 2025/0188405 A1
(43) Pub. Date: Jun. 12, 2025

(54) SINGLE CELL PROTEIN WITH ENHANCED DIGESTIVE ENZYME CONTENT

(71) Applicant: LanzaTech, Inc., Skokie, IL (US)

(72) Inventors: James Clomburg, Chicago, IL (US); Sean Simpson, Auckland (NZ); Alex Chou, Lincolnwood, IL (US); Natalie Gayner, Chicago, IL (US); Stephanie Jones, Evanston, IL (US); Timothy Politano, Chicago, IL (US)

(21) Appl. No.: 18/977,438

(22) Filed: Dec. 11, 2024

Related U.S. Application Data
(60) Provisional application No. 63/608,786, filed on Dec. 11, 2023.

(51) Int. Cl. C12N 1/20; C12N 9/16; C12N 9/54

(52) U.S. Cl. CPC C12N 1/20; C12N 9/16; C12N 9/54; C12Y 304/21062

(57) ABSTRACT

The present disclosure relates to single cell protein (SCP) products obtained from Cupriavidus necator that has been engineered to produce digestive enzymes or otherwise enhanced. The biomass produced by the C. necator and the SCP products that include it contain digestive enzymes that are not naturally produced by C. necator.

### CROSS REFERENCE TO RELATED APPLICATIONS

This application claims the benefit of U.S. Provisional Patent Application No. 63/608,786 filed on Dec. 11, 2023, the entirety of which is incorporated herein by reference.

### REFERENCE TO A SEQUENCE LISTING

The application contains a Sequence Listing which has been submitted electronically in ST.26 Sequence listing XML format and is hereby incorporated by reference in its entirety. Said ST.26 Sequence listing XML, created on Nov. 12, 2024, is named LT294US1-Sequences.xml and is 7,384,282 bytes in size.

### BACKGROUND

The following discussion is merely provided to aid the reader in understanding the disclosure and is not admitted to describe or constitute prior art thereto.

Cupriavidus necator is a type of bacteria commonly used for the production of single cell protein for food and feed applications. It is generally high in protein content with a favorable amino acid profile. However, some components of single cell protein compositions may be difficult to digest for some animals or humans.

### SUMMARY

The present disclosure provides nutritionally enhanced single cell protein (SCP) that includes digestive enzymes.

In one aspect, the present disclosure provides strains of Cupriavidus necator, comprising a transgene encoding a heterologous digestive enzyme, wherein the strain of C. necator synthesizes at least one digestive enzyme that is not natively produced by C. necator. The at least one digestive enzyme may be selected from a protease, a carbohydrase, a lipase, a phytase, and any combination thereof.

In another aspect, the C. necator strain comprises a second transgene encoding a second heterologous digestive enzyme selected from a protease, a carbohydrase, a lipase, a phytase, and any combination thereof. In yet another aspect, the C. necator strain comprises a third transgene encoding a second heterologous digestive enzyme selected from a protease, a carbohydrase, a lipase, a phytase, and any combination thereof.

In another aspect, the present disclosure provides a C. necator strain that expresses one or of more heterologous digestive enzymes selected from a protease, a carbohydrase, a lipase, a phytase, and any combination thereof. In some aspects, the C. necator strain expresses at least two heterologous digestive enzymes selected from a protease, a carbohydrase, a lipase, a phytase, and any combination thereof. In yet another aspect, the C. necator strain expresses at least three heterologous digestive enzymes selected from a protease, a carbohydrase, a lipase, a phytase, and any combination thereof.

For the purposes of the present disclosure, the protease may include a serine protease, a cysteine protease, or an aspartic protease. The carbohydrase is a cellulase, a β-glucanase, a xylanase, a mannanase, an amylase, a pectinase, a glucosyltransferase, or a galactosidase.

The heterologous digestive enzymes of the present disclosure may be from a bacteria, a fungi, a cyanobacteria, an archaeum, or a eukaryote. The bacteria may be a Bacillus species (sp), Bifidobacterium species (sp), Caldalkalibacillus species (sp), Caldicellulosiruptor species (sp), Cellvibrio species (sp), Clostridium species (sp), Cupriavidus species (sp), Rhodothermus species (sp), Streptomyces species (sp), Vibrio species (sp), Acidothermus species (sp), butyrivibrio species (sp), Cellulomonas species (sp), Dickeya species (sp), Escherichia species (sp), Fibrobacter species (sp), Halalkalibacter species (sp), Martelella species (sp), Novacetimonas species (sp), Paenibacillus species (sp), Pectobacterium species (sp), Ralstonia species (sp), Ruminiclostridium species (sp), Ruminococcus species (sp), Salipaludibacillus species (sp), Thermoanaerobacterium species (sp), Thermobifida species (sp), Thermoclostridium species (sp), Xanthomonas species (sp), Pseudothermotoga species (sp), Citrobacter species (sp). The Bacillus species may be selected from Bacillus subtilis, Bacillus licheniformis, Bacillus amyloliquefaciens, and Bacillus lentus. The archaeum may be a Thermococcus species. The eukaryote may be a Aspergillus species (sp), Hypocrea species (sp), Candida species (sp), Saccharomyces species (sp), Fusarium species (sp), or a Phanerodontia species (sp).

The transgene(s) used for the purposes of any of the disclosed aspects or embodiments may be codon optimized for expression in Cupriavidus necator.

For the purposes of the present disclosure, the strain of C. necator may be modified by, for example, adaptive laboratory evolution or genetic modification.

For the purposes of the present disclosure, the strain of C. necator may be a strain that grows autotrophically at up to 40° C. For example, the strain of C. necator may be the strain deposited at DSM34774. Additionally or alternatively, the strain of C. necator may have a H₂:CO₂ uptake ratio that is lower than the H₂:CO₂ uptake ratio of a wild type or naturally occurring strain of C. necator cultured under corresponding temperatures and conditions. In some embodiments, the strain of C. necator may comprise a full or partial deletion of a gene or locus encoding a membrane-bound hydrogenase, such as hoxKGXZ.

The strain of C. necator may also be a strain that produces high levels of protein, which has a favorable amino acid profile. For example, the strain of C. necator may produce about 40%, about 50%, about 60%, about 70%, about 80%, about 90%, or about 95% by weight of protein.

The heterologous digestive enzyme(s) expressed by the disclosed strains of C. necator may be selected from a protease, a carbohydrase, a lipase, a phytase, and any combination thereof. Such heterologous digestive enzymes can be from a bacteria, such as a bacillus species (sp), or a eukaryote, such as a yeast.

In another aspect, the present disclosure provides methods of preparing a single-cell protein product, comprising:
(a) culturing a strain of C. necator disclosed herein (e.g., any of the foregoing aspects or embodiments), thereby creating biomass comprising protein and digestive enzyme,
(b) isolating the C. necator or the biomass, and
(c) preparing a single-cell protein product from the C. necator or the biomass.

In another aspect, the present disclosure provides methods of producing a digestive enzyme, comprising culturing a strain of C. necator disclosed herein (e.g., any of the foregoing aspects or embodiments) such that the digestive enzyme is produced. In some embodiments, the methods may further comprise isolating the digestive enzyme or isolating the strain of C. necator.

Culturing may comprise growing the strain of C. necator in a fermentation tank. The fermentation tank can be a gas fermentation tank.

Culturing may also comprise growing the strain of C. necator in autotrophic conditions. The autotrophic conditions comprise providing carbon monoxide (CO) or carbon dioxide (CO₂) as a carbon source. Hydrogen gas may also be present.

Culturing may also comprise growing the strain of C. necator at a temperature above 30° C. and up to 40° C. For example, the temperature of the culture may be 31° C., 32° C., 33° C., 34° C., 35° C., 36° C., 37° C., 38° C., 39° C., or 40° C.

In another aspect, the present disclosure provides single-cell protein (SCP) compositions, comprising a strain of C. necator disclosed herein (e.g., any of the foregoing aspects or embodiments) or biomass created therefrom.

The composition may comprise at least about 40%, about 50%, about 60%, about 70%, about 80%, about 90%, or about 95% by weight of protein.

The composition may comprise at least about 40%, about 50%, about 60%, about 70%, about 80%, about 90%, or about 95% by weight of a digestive enzyme component. The digestive enzyme component may comprise a protease, a carbohydrase, a lipase, a phytase, and any combination thereof.

The composition can be produced by culturing a strain of C. necator disclosed herein, thereby creating biomass comprising protein and digestive enzyme, and isolating the C. necator or the biomass.

In another aspect, the present disclosure provides edible products, comprising (a) a strain of C. necator disclosed herein or biomass created therefrom, or (b) an SCP composition disclosed herein.

The edible product may be an animal feed, such as a fish feed, a livestock or ruminant feed, a swine feed, goat feed, llama feed, turkey feed, a poultry feed, a rodent feed, a dog feed, and a cat feed.

The edible product may be a human food, such as a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.

In another aspect, the present disclosure provides method of preparing an edible product, comprising mixing (a) a strain of C. necator disclosed herein or biomass created therefrom, or (b) an SCP composition of C. necator disclosed herein with one or more edible ingredients.

In another aspect, the present disclosure provides topical product, comprising a digestive enzyme produced by a method disclosed herein or from a strain of C. necator disclosed herein. The topical product may be, for example, a lotion, balm, cream, or makeup.

The foregoing general description and following detailed description are examples and are intended to provide further explanation of the disclosure as claimed. Other objects, advantages, and novel features will be readily apparent to those skilled in the art from the following brief description of the drawings and detailed description of the disclosure.

It should be appreciated that all combinations of the foregoing concepts and additional concepts discussed in greater detail below are provided as being part of the inventive subject matter disclosed herein and may be employed in any combination to achieve the benefits described herein.

### BRIEF DESCRIPTION OF DRAWINGS

These and other aspects of the present disclosure, which should be considered in all its novel aspects, will become apparent from the following description, which is given by way of example only, with reference to the accompanying figures, in which:

FIG. 1 (FIG. 1): Biomass titer (OD600) and reactor temperature throughout the ˜100 day temperature-tolerance evolution of DSM 541 in CSTR. Reactor inputs were kept consistent throughout the experiment, while reactor temperature was gradually increased step-wise.

FIG. 2 (FIG. 2): Biomass titer (OD600) and reactor temperature of a representative autotrophic CSTR run with DSM 34774. Reactor temperature was held constant at 37° C. demonstrating the ability of the temperature evolved strain to grow on CO₂/H₂/O₂ gas mixtures at elevated temperatures.

FIG. 3 (FIG. 3): Expression of protease enzymes in Cupriavidus necator. Protease expression in total cell lysate of synthetic constructs with HiBiT tags detected using HiBiT Lytic Detection System. Genes encoding proteases and linked HiBiT tags were expressed via rhamnose inducible promoter with significant expression detection upon addition of 0.1 mM rhamnose.

FIGS. 4A-4C (FIGS. 4A-4C): show open reading frames (ORFs) in the Δ35433 bp encompassing genes [E6Δ55_33395]-[E6Δ55_33560] of the C. necator genome.

FIG. 5 (FIG. 5): shows the expression and activity of phytase enzymes in Cupriavidus necator.

FIG. 6 (FIG. 6): shows the expression of phytase enzymes in Cupriavidus necator.

### DETAILED DESCRIPTION

The disclosure provides an engineered C. necator bacteria that a heterologous digestive enzyme that is not natively produced by C. necator. The bacteria may be a novel strain of heat-adapted Cupriavidus necator bacteria.

### I. Definitions

It is to be understood that the disclosed compositions and methods are not limited to the particular implementations described, and as such may vary. It is also to be understood that the terminology used herein is for the purpose of describing particular implementations only, and is not intended to be limiting. The scope of the present technology will be limited only by the appended claims.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “about” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself. Thus, by way of example, “about 10” should be understood to mean “10” and a range no larger than “9-11”.

As used herein, the term “bioproduction” is intended to mean production of a compound by way of biological or enzymatic synthesis (as opposed to chemical synthesis). In some implementations, bioproduction may be performed by a transgenic organism or microbe (e.g., C. necator) that has been engineered to express enzymes involved in the biological synthesis of a compound of interest (e.g., a digestive enzymes).

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “protein” is a biological macromolecule comprised of one or more chain(s) of amino acids. An “enzyme” is a type of protein that possesses a biological catalytic activity that accelerates chemical reaction. Thus, for the purposes of this disclosure, enzymes are an example of a protein that can catalyze a reaction, such as digestion or cleavage of another protein or macromolecule.

The terms “engineered cell” or “engineered host cell” refer to a modified cell wherein the modification can be selected from e.g., increased expression of a gene, inhibited expression of a gene, knockout of a gene or genes, introduction of a new gene or genes, introduction of mutant gene(s), or mutation/genetic alteration of gene(s), wherein the increased expression or inhibited expression of a gene can be achieved by using techniques, such as gene deletion, changed gene copy number, changed gene promoter (e.g. by using a strong or weak promoter), etc. An engineered cell or engineered host cell may also include a cell that has been isolated. In some implementations, an engineered cell or engineered host cell is a transgenic cell. In some implementations, an engineered cell or engineered host cell is a transgenic cell capable of producing high levels of a compound or biomolecule of interest. An example of a host cell herein may be a C. necator cell.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The terms “derived from” or “derivative” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes.

For the purposes of this disclosure, all of the compounds, enzymes, and cells disclosed herein can be isolated in a form that is substantially free of other proteins, contaminants, macromolecules (e.g., nucleic acids, lipids, etc.), or cells. However, it should be understood that an “isolated” enzyme may not be 100% free of other proteins, contaminants, or macromolecules, and absolute purity is not required in order for a protein or enzyme to be considered “isolated.”

For the purposes of this disclosure, a “wild type” is the phenotype or sequence of the typical form of a cell, protein, or enzyme as it occurs in nature (i.e., the “normal” or “standard” cell or protein sequence, as opposed to an engineered or otherwise altered variant or a naturally occurring mutant).

As used herein, “optional” or “optionally” means that the subsequently described event or circumstance may or may not occur, and that the description includes instances where said event or circumstance occurs and instances where it does not.

The term “metabolic trait” refers to metabolic features of the cell or organisms, e.g. the capability to produce a certain product under certain conditions, the capability to survive and reproduce under certain conditions, such as certain pH, temperature, certain gas levels, or certain nutrients, preferably certain temperatures or certain nutrients.

As used herein, the term “vector” refers to a nucleic acid molecule that can transport another nucleic acid to which it is attached. One type of vector is a “plasmid,” which stands for a circular double-stranded DNA loop into which additional DNA segments can be ligated.

Another type of vector is a viral vector, whereby additional DNA segments can be ligated into the viral genome. Certain vectors can replicate autonomously in a host cell into which they have been introduced (e.g., bacterial vectors with a bacterial origin of replication). Other vectors are advantageously integrated into the genome of a host cell when introduced into the host cell and are thereby replicated together with the host genome. In addition, certain vectors can control the expression of genes to which they are operably linked. These vectors are referred to here as “expression vectors.” Usually expression vectors suitable for recombinant DNA techniques are in the form of plasmids. In the present description, “plasmid” and “vector” can be used interchangeably because the plasmid is the most commonly used vector form. However, the disclosure is intended to encompass these other expression vector forms, such as viral vectors, which perform similar functions.

“Transgene” or “recombinant” as used in the present disclosure, for example with respect to a nucleic acid sequence, an expression cassette, gene construct, or a vector containing the nucleic acid sequence according to the disclosure or an organism transformed with the nucleic acid sequences, expression cassette or vector according to the disclosure, all those obtained by genetic engineering methods Constructions in which either a) the nucleic acid sequence according to the disclosure, or b) a genetic control sequence functionally linked to the nucleic acid sequence according to the disclosure, for example a promoter, or c) (a) and (b) are not in their natural, genetic environment or were modified by genetic engineering methods, the modification being an example.

A transgenic organism or transgenic bacteria in the sense of the disclosure is to be understood to mean that the nucleic acids used in the method do not bind their natural position in the genome of an organism, the nucleic acids can be expressed homologously or heterologously. However, as mentioned, transgene also means that the nucleic acids according to the disclosure are in their natural place in the genome of an organism, but that the sequence has been changed compared to the natural sequence and/or that the regulatory sequences of the natural sequences have been changed. Transgenic is preferably to be understood as meaning the expression of the nucleic acids according to the disclosure at a non-natural location in the genome, that is to say that the nucleic acids are homologous or preferably heterologous.

As used herein, the term “autotrophic bacteria” refers to a bacteria that is capable of producing all its biomass carbon from CO₂.

For the purpose of the description, a phrase in the form “A/B” or in the form “A and/or B” means (A), (B), or (A and B).

### II. C1-Fixing Microorganisms

As described above, however, the microorganism of the disclosure may also be derived from essentially any parental microorganism, such as a parental microorganism selected from the group consisting of Escherichia coli and Saccharomyces cerevisiae

In another embodiment, the microorganism of the disclosure is an aerobic bacterium. In one embodiment, the microorganism of the disclosure comprises aerobic hydrogen bacteria. In an embodiment, the aerobic bacteria comprising at least one disrupted gene.

A number of aerobic bacteria are known to be capable of carrying out fermentation for the disclosed methods and system. Examples of such bacteria that are suitable for use in the disclosure include bacteria of the genus Cupriavidus and Ralstonia. In some embodiments, the aerobic bacteria is Cupriavidus necator or Ralstonia eutropha. In some embodiments, the aerobic bacteria is Cupriavidus alkaliphilus. In some embodiments, the aerobic bacteria is Cupriavidus basilensis. In some embodiments, the aerobic bacteria is Cupriavidus campinensis. In some embodiments, the aerobic bacteria is Cupriavidus gilardii. In some embodiments, the aerobic bacteria is Cupriavidus laharis. In some embodiments, the aerobic bacteria is Cupriavidus metallidurans. In some embodiments, the aerobic bacteria is Cupriavidus nantongensis. In some embodiments, the aerobic bacteria is Cupriavidus numazuensis. In some embodiments, the aerobic bacteria is Cupriavidus oxalaticus. In some embodiments, the aerobic bacteria is Cupriavidus pampae. In some embodiments, the aerobic bacteria is Cupriavidus pauculus. In some embodiments, the aerobic bacteria is Cupriavidus pinatubonensis. In some embodiments, the aerobic bacteria is Cupriavidus plantarum. In some embodiments, the aerobic bacteria is Cupriavidus respiraculi. In some embodiments, the aerobic bacteria is Cupriavidus taiwanensis. In some embodiments, the aerobic bacteria is Cupriavidus yeoncheonensis.

In some embodiments, the strain is Cupriavidus necator DSM 428, DSM 531, or DSM541, or any derivatives thereof. In another embodiment, the strain is Cupriavidus necator DSM 34774. In some embodiments, the strain comprises SEQ ID NOs: 1-3. In some embodiments, the strain is a derivative of Cupriavidus necator DSM 34774. In another embodiment, the strain is a derivative of SEQ ID NO: 1-3.

In some embodiments, the aerobic bacteria comprises one or more exogenous nucleic acid molecules encoding a naturally occurring polypeptide, wherein the polypeptide is ribulose bisphosphate carboxylase, acetyl-CoA acetyltransferase, 3-hydroxybutyryl-CoA dehydratase, butyryl-CoA dehydrogenase, butanol dehydrogenase, electron-transferring flavoprotein large subunit, 3-hydroxybutyryl-CoA dehydrogenase, bifunctional acetaldehyde-CoA/alcohol dehydrogenase, acetaldehyde dehydrogenase, aldehyde decarbonylase, acyl-ACP reductase, L-1,2-propanediol oxidoreductase, acyltransferase, 3-oxoacyl-ACP synthase, 3-hydroxybutyryl-CoA epimerase/delta(3)-cis-delta(2)-trans-enoyl-CoA isomerase/enoyl-CoA hydratase/3-hydroxyacyl-CoA dehydrogenase, short chain dehydrogenase, trans-2-enoyl-CoA reductase, or any combination thereof.

In the microorganisms of the disclosure, carbon flux is strategically diverted away from nonessential or undesirable products and towards products of interest. In certain embodiments, these disrupted genes divert carbon flux away from nonessential or undesirable metabolic nodes and through target metabolic nodes to improve production of products downstream of those target metabolic nodes. In an embodiment, limitation selected from nutrients, dissolved oxygen, or any combination thereof diverts carbon flux to desired products.

In an embodiment, the fermentation broth comprises the feed streams in combination with the aerobic microorganism in the bioreactor. In some embodiments, the feed streams, e.g., a carbon source feed stream, a flammable gas-containing stream, and an oxygen-containing gas feed stream, react with the microorganism in the bioreactor to at least partially form the fermentation broth (which may also include other products, byproducts, and other media fed to the bioreactor). The unreacted oxygen, or the oxygen that is not consumed by the microorganism, exists as both dissolved oxygen and gaseous oxygen in a dispersed gaseous phase within the fermentation broth. The same holds true for the other gases that are soluble. The dispersed gaseous phase, containing the unreacted components, e.g., oxygen, nitrogen, hydrogen, carbon dioxide and/or water vapor, rises to the headspace of the bioreactor.

In some embodiments, an oxygen-containing gas, e.g., air, can be fed directly into the fermentation broth. In one embodiment, the oxygen-containing gas can be an oxygen-enriched source, e.g., oxygen-enriched air or pure oxygen. In an embodiment, the oxygen-containing gas may comprise greater than 6.0 vol. % of oxygen, e.g., greater than 10.0 vol. %, greater than 20.0 vol. %, greater than 40.0 vol. %, greater than 60.0 vol. %, greater than 80.0 vol. %, or greater than 90.0 vol. %. In some embodiments, the oxygen-containing gas may be pure oxygen.

In one embodiment, the microorganism of the disclosure is capable of producing ethylene. One embodiment is directed to a recombinant C1-fixing microorganism capable of producing ethylene from a carbon source comprising a nucleic acid encoding a group of exogenous enzymes comprising at least one ethylene forming enzyme (EFE). In some embodiments the EFE is derived from Pseudomonas syringae. In an embodiment, the EFE has an E.C. number 1.13.12.19. The microorganism of an embodiment comprising at least one EFE having an E.C. number 1.13.12.19. The microorganism of an embodiment, further comprising a nucleic acid encoding a group of exogenous enzymes comprising at least one alpha-ketoglutarate permease (AKGP).

The microorganism of an embodiment, wherein a nucleic acid encoding a group of exogenous enzymes comprises at least one EFE, at least one AKGP, or any combination thereof. The microorganism of an embodiment, wherein a nucleic acid encoding a group of exogenous enzymes comprises at least one EFE and at least one AKGP. The microorganism of an embodiment, wherein the nucleotide encoding a group of exogenous enzymes is inserted into a bacterial vector plasmid, a high copy number bacterial vector plasmid, a bacterial vector plasmid having an inducible promoter, a nucleotide guide of a homologous recombination system, a CRISPR Cas system, or any combination thereof. In an embodiment, the promoter is a phosphate limited inducible promoter. In some embodiments, the promoter is a nitrogen limited promoter. In some embodiments, the promoter is an NtrC-P activated promoter. In some embodiments, the promoter is a H₂ inducible promoter. In one embodiment, the microorganism comprises an intracellular oxygen concentration limit. In another embodiment, the method limits intracellular oxygen concentration. In one embodiment, the method comprises a step of controlling dissolved oxygen. In an embodiment, the method comprises decreased ethylene production with decreased dissolved oxygen concentration. In some embodiments, the microorganism comprises a molecular switch. In some embodiments, the microorganism comprises an ability to switch the cellular burden under variable conditions.

In some embodiments, the microorganism is a natural or an engineered microorganism that is capable of converting a gaseous substrate as a carbon and/or energy source. In one embodiment, the gaseous substrate includes CO₂ as a carbon source. In some embodiments, the gaseous substrate includes H₂, and/or O₂ as an energy source. In one embodiment, the gaseous substrate includes a mixture of gases, comprising H₂ and/or CO₂ and/or CO.

In some embodiments, the gas fermentation product is selected from an alcohol, an acid, a diacid, an alkene, a terpene, an isoprene, and alkyne. In some embodiments, the method and microorganism disclosed herein are for the improved production of ethylene. In an embodiment, the method and microorganism disclosed herein are for the improved production of a gas fermentation product.

In one embodiment, the aerobic bacteria may produce a product such as acetone, isopropanol, 3-hydroxyisovaleryl-CoA, 3-hydroxyisovalerate, isobutylene, isopentenyl pyrophosphate, dimethylallyl pyrophosphate, isoprene, farnesene, 3-hydroxybutyryl-CoA, crotonyl-CoA, 3-hydroxybutyrate, 3-hydroxybutyrylaldehyde, 1,3-butanediol, 2-hydroxyisobutyryl-CoA, 2-hydroxyisobutyrate, butyryl-CoA, butyrate, butanol, caproate, hexanol, octanoate, octanol, 1,3-hexanediol, 2-buten-1-ol, isovaleryl-CoA, isovalerate, isoamyl alcohol, methacrolein, methyl-methacrylate, or any combination thereof.

In another embodiment, the bacteria of the disclosure may produce ethylene, ethanol, propane, acetate, 1-butanol, butyrate, 2,3-butanediol, lactate, butene, butadiene, methyl ethyl ketone (2-butanone), acetone, isopropanol, a lipid, 3-hydroxypropionate (3-HP), a terpene, isoprene, a fatty acid, 2-butanol, 1,2-propanediol, 1-propanol, 1-hexanol, 1-octanol, a fatty alcohol, chorismate-derived products, 3-hydroxybutyrate, 1,3-butanediol, 2-hydroxyisobutyrate or 2-hydroxyisobutyric acid, isobutylene, adipic acid, keto-adipic acid, 1,3hexanediol, 3-methyl-2-butanol, 2-buten-1-ol, isovalerate, isoamyl alcohol, and monoethylene glycol, or any combination thereof.

The disclosure provides microorganisms capable of producing ethylene comprising culturing the microorganism of the disclosure in the presence of a substrate, whereby the microorganism produces ethylene.

As used herein, the terms “intermediate” and “precursor” can be used interchangeably to refer to a substance, such as a molecule, compound, or protein, that is produced upstream of a particular product. The intermediate may be directly upstream of the product. The intermediate may be indirectly upstream of the product. For example, in the exemplary reaction “compound A”→“compound B”→“compound C”→“compound D”, “compound” C is an intermediate that is directly upstream of the product, “compound D,” and “compound B” is an intermediate that is indirectly upstream of the product, “compound D.”

The enzymes of the disclosure may be codon optimized for expression in the microorganism of the disclosure. “Codon optimization” refers to the mutation of a nucleic acid, such as a gene, for optimized or improved translation of the nucleic acid in a particular strain or species. Codon optimization may result in faster translation rates or higher translation accuracy. In a preferred embodiment, the genes of the disclosure are codon optimized for expression in the microorganism of the disclosure. Although codon optimization refers to the underlying genetic sequence, codon optimization often results in improved translation and, thus, improved enzyme expression. Accordingly, the enzymes of the disclosure may also be described as being codon optimized.

One or more of the enzymes of the disclosure may be overexpressed. “Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The enzymes of the disclosure may comprise a disruptive mutation. A “disruptive mutation” refers to a mutation that reduces or eliminates (i.e., “disrupts”) the expression or activity of a gene or enzyme. The disruptive mutation may partially inactivate, fully inactivate, or delete the gene or enzyme. The disruptive mutation may be a knockout (KO) mutation. The disruptive mutation may be any mutation that reduces, prevents, or blocks the biosynthesis of a product produced by an enzyme. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

The disclosed microorganisms, including C. necator, may utilize various carbon sources, including gaseous carbon sources as a substrate.

In some embodiments, the substrate comprises CO₂ and an energy source. In some embodiments, the substrate comprises CO₂ and an energy source. In an embodiment, the substrate comprises CO₂, H₂, and O₂. In some embodiments, the substrate comprises CO₂ and any suitable energy source. In one embodiment, the substrate comprises CO. In one embodiment, the substrate comprises CO₂ and CO. In another embodiment, the substrate comprises CO₂ and H₂. In another embodiment, the substrate comprises CO₂ and CO and H₂.

“Substrate” refers to a carbon and/or energy source for the microorganism of the disclosure. Often, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons. In other embodiments, however, the substrate may be a carbohydrate, such as sugar, starch, fiber, lignin, cellulose, or hemicellulose or a combination thereof. For example, the carbohydrate may be fructose, galactose, glucose, lactose, maltose, sucrose, xylose, or some combination thereof. In some embodiments, the substrate does not comprise (D)-xylose (Alkim, Microb Cell Fact, 14: 127, 2015). In some embodiments, the substrate does not comprise a pentose such as xylose (Pereira, Metab Eng, 34: 80-87, 2016). In some embodiments, the substrate may comprise both gaseous and carbohydrate substrates (mixotrophic fermentation). The substrate may further comprise other non-carbon components, such as H₂, N₂, or electrons.

In some embodiments, the gaseous substrate generally comprises at least some amount of CO, such as about 1, 2, 5, 10, 20, 30, 40, 50, 60, 70, 80, 90, or 100 mol % CO. The gaseous substrate may comprise a range of CO, such as about 20-80, 30-70, or 40-60 mol % CO. Preferably, the gaseous substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the gaseous substrate may comprise a relatively low amount of CO, such as about 1-10 or 1-20 mol % CO. The microorganism of the disclosure typically converts at least a portion of the CO in the gaseous substrate to a product. In some embodiments, the gaseous substrate comprises no or substantially no (<1 mol %) CO.

The gaseous substrate may comprise some amount of H₂. For example, the gaseous substrate may comprise about 1, 2, 5, 10, 15, 20, or 30 mol % H₂. In some embodiments, the gaseous substrate may comprise a relatively high amount of H₂, such as about 60, 70, 80, or 90 mol % H₂. In further embodiments, the gaseous substrate comprises no or substantially no (<1 mol %) H₂.

The gaseous substrate may comprise some amount of CO₂. For example, the gaseous substrate may comprise about 1-80 or 1-30 mol % CO₂. In some embodiments, the gaseous substrate may comprise less than about 20, 15, 10, or 5 mol % CO₂. In another embodiment, the gaseous substrate comprises no or substantially no (<1 mol %) CO₂.

The gaseous substrate may also be provided in alternative forms. For example, the gaseous substrate may be dissolved in a liquid or adsorbed onto a solid support.

The gaseous substrate and/or C1-carbon source may be a waste gas or an off gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the gaseous substrate and/or C1-carbon source may be captured from the industrial process before it is emitted into the atmosphere, using any convenient method.

The gaseous substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The substrate and/or C1-carbon source may be a waste gas obtained as a byproduct of an industrial process or from another source, such as automobile exhaust fumes, biogas, landfill gas, direct air capture, or from electrolysis. The substrate and/or C1-carbon source may be syngas generated by pyrolysis, torrefaction, or gasification. In other words, carbon in waste material may be recycled by pyrolysis, torrefaction, or gasification to generate syngas which is used as the substrate and/or C1-carbon source. The substrate and/or C1-carbon source may be a gas comprising methane.

In certain embodiments, the industrial process is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, geological reservoirs, gas from fossil resources such as natural gas coal and oil, or any combination thereof.

Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, or gasification processes. Examples of gasification processes include gasification of coal, gasification of refinery residues, gasification of petroleum coke, gasification of biomass, gasification of lignocellulosic material, gasification of waste wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of biogas. Examples of reforming processes include, steam methane reforming, steam naphtha reforming, reforming of natural gas, reforming of biogas, reforming of landfill gas, naphtha reforming, and dry methane reforming. Examples of partial oxidation processes include thermal and catalytic partial oxidation processes, catalytic partial oxidation of natural gas, partial oxidation of hydrocarbons. Examples of municipal solid waste include tires, plastics, fibers, such as in shoes, apparel, and textiles. Municipal solid waste may be simply landfill-type waste. The municipal solid waste may be sorted or unsorted. Examples of biomass may include lignocellulosic material and may also include microbial biomass. Lignocellulosic material may include agriculture waste and forest waste.

The substrate and/or C1-carbon source may be a gas stream comprising methane. Such a methane containing gas may be obtained from fossil methane emission such as during fracking, wastewater treatment, livestock, agriculture, and municipal solid waste landfills. It is also envisioned that the methane may be burned to produce electricity or heat, and the C1 byproducts may be used as the substrate or carbon source.

The composition of the gaseous substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

Regardless of the source or precise content of the gas used as a feedstock, the feedstock may be metered (e.g., for carbon credit calculations or mass balancing of sustainable carbon with overall products) into a bioreactor in order to maintain control of the follow rate and amount of carbon provided to the culture. Similarly, the output of the bioreactor may be metered (e.g., for carbon credit calculations or mass balancing of sustainable carbon with overall products) or comprise a valved connection that can control the flow of the output and products (e.g., ethylene, ethanol, acetate, 1-butanol, etc.) produced via fermentation. Such a valve or metering mechanism can be useful for a variety of purposes including, but not limited to, slugging of product through a connected pipeline and measuring the amount of output from a given bioreactor such that if the product is mixed with other gases or liquids the resulting mixture can later be mass balanced to determine the percentage of the product that was produced from the bioreactor.

In certain embodiments, the fermentation is performed in the absence of carbohydrate substrates, such as sugar, starch, fiber, lignin, cellulose, or hemicellulose.

A microorganism of the present disclosure may classified based on functional characteristics. For example, the microorganism may be or may be derived from a C1-fixing microorganism, an aerobe, a hydrogen-oxidizing bacteria, a hydrogenotroph, an anaerobe, an acetogen, an ethanologen, and/or a carboxydotroph.

III. Cupriavidus necator

Cupriavidus necator (also referred to as Hydrogenomonas eutrophus, Alcaligenes eutropha, Ralstonia eutropha, and Wautersia eutropha) is a Gram-negative, flagellated soil bacterium of the Betaproteobacteria class. It is a hydrogen-oxidizing bacterium that is capable of growing in both anaerobic and aerobic environments.

In addition to wild-type and naturally occurring strains of C. necator, the present disclosure also provides non-naturally occurring C. necator strains that are capable of growing in autotrophic conditions at elevated temperatures (i.e., temperature above 30° C.). Such temperature-resistant strains of C. necator are capable of growth even up to 40° C. Naturally occurring strains of C. necator are incapable of growth and survival at temperatures above 30° C. Thus, for the purposes of the present disclosure, the C. necator strain that is engineered to produce digestive enzymes can be a strain that can go in autotrophic conditions between 31-40° C. In other words, the engineered C. nectar of the present disclosure can be prepared from a strain that can grow autotrophically at 31° C., 32° C., 33° C., 34° C., 35° C., 36° C., 37° C., 38° C., 39° C., or at 40° C. The C. nectar may grow at a range of 32-40° C., 33-40° C., 34-40° C., 35-40° C., 31-39° C., 32-39° C., 33-39° C., 34-39° C., or 35-39° C.

The disclosed temperature resistant strains of C. necator were prepared without genetic modification using Adaptive Laboratory Evolution (ALE), as detailed further in Example 1. Briefly, the bacteria is continuously cultivated under clearly defined conditions for prolonged periods of time. The strain deposited at DSM 34774 possesses the desired phenotype characterized by increased temperature tolerance and surprising growth in autotrophic conditions obtained through ALE. However, such temperature resistant strains could feasibly be prepared through genetic modification as well. SEQ ID NOs: 1-3 provide the genome of an ALE-derived strain of C. necator that is temperature resistant and able to grow in autotrophic conditions at up to 40° C., and those skilled in the art could use this information to prepare further temperature resistant strains via genetic modification of a wild-type strain of C. necator. Such temperature resistant C. necator may be used as a chassis or host cell for the introduction of heterologous genes from other species.

C. necator strains of present disclosure, including both naturally occurring strains or temperature resistant strains, provide several process advantages for the production of single cell protein for food and feed applications as well as other products. The strains are high in protein content with a favorable amino acid profile, and the disclosed strains can be integrated various processing systems, including processing systems that require increased heat integration schemes and/or limited processing reagents, with relative ease. Further, the C. necator bacteria provided herein may be advantageously capable of culture and growth in elevated temperature conditions that may be required for enzyme function, biosynthesis, or any metabolic process that requires elevated temperatures exceeding the tolerance of naturally occurring C. necator bacteria strains.

For the purposes of the present disclosure, the C. necator strain utilized for producing digestive enzymes may be a wild-type or naturally occurring stain that is engineered to express one or more digestive enzymes, including, but not limited to, a protease, a carbohydrase, a lipase, a phytase, and any combination thereof. Alternatively, the C. necator strain utilized for producing the digestive enzymes may optionally be a non-naturally occurring strain, such as a heat adapted or temperature resistant strain of C. necator that can grow at temperatures above 30° C. As explained herein, such heat adapted or temperature resistant strains may be obtained by genetic modification or by ALE. Additionally or alternatively, the C. necator strain utilized for producing digestive enzymes may optionally be modified-either via ALE or genetic modification—to minimize the H₂:CO₂ uptake ratio while maximizing intracellular O₂ during autotrophic growth.

For the purposes of the present disclosure, the C. necator strain utilized for producing digestive enzymes may have a genome comprising or consisting of the nucleic acid sequence of SEQ ID NOs: 1-3 or a genome comprising or consisting of a nucleic acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NOs: 1-3. Additionally or alternatively, the C. necator strain utilized for producing digestive enzymes may have a genome comprising or consisting of a nucleic acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% similarity to SEQ ID NOs: 1-3. The C. necator strain utilized for producing digestive enzymes may also be the strain deposited under accession number DSM 34774, or a strain sharing substantial phenotypic characteristics (e.g., temperature resistance) or genotypic characteristics (e.g., a genome with at least 80% sequence identity to SEQ ID NOs: 1-3 or a genome with at least 80% sequence similarity to SEQ ID NOs: 1-3) with a strain deposited under accession number DSM 34774.

The C. necator strain having identification reference number DSM 34774 was deposited under the provisions of the Budapest Treaty at the German Collection of Microorganisms (DSM) located at Inhoffenstraße 7B, 38124 Braunschweig, Science Campus Braunschweig-Süd, Germany on Oct. 6, 2023. All restrictions on the availability to the public of the deposited material will be irrevocably removed upon the granting of a patent from the above-identified application. The deposited cultures will be replaced should they die or be destroyed during the enforceable life of any patent issued out of this patent application, for five years after the last request for a sample of the deposited microorganism or for a term of at least thirty (30) years. Samples will be stored under agreements that would make them available beyond the enforceable life of the patent for which the deposit was made.

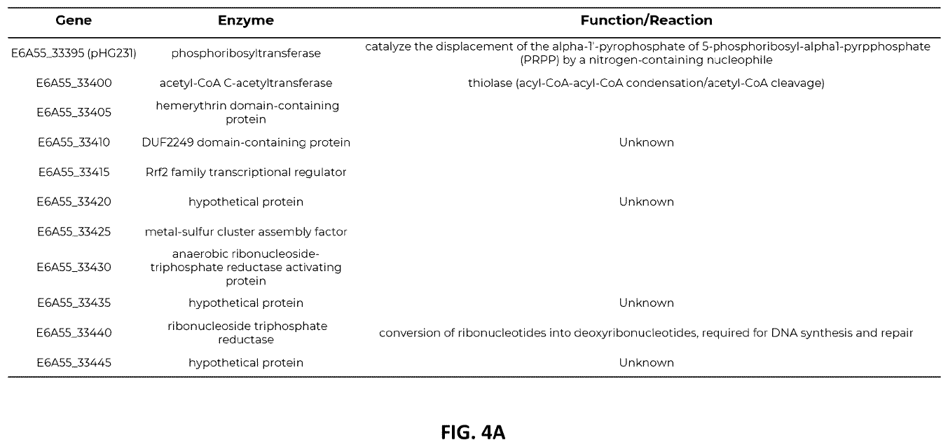

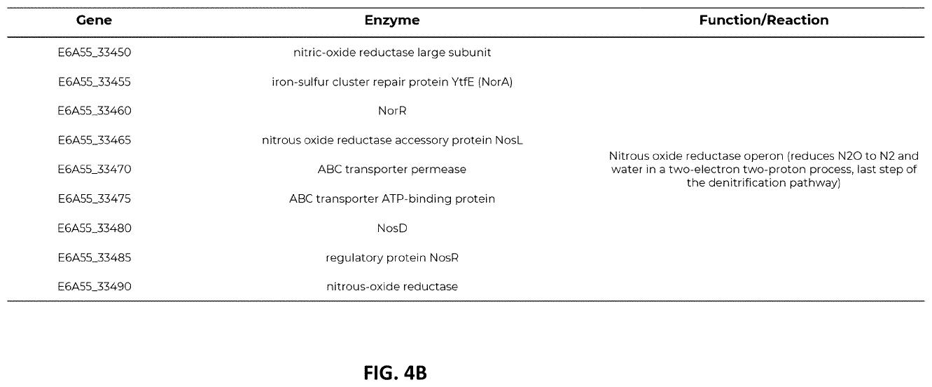

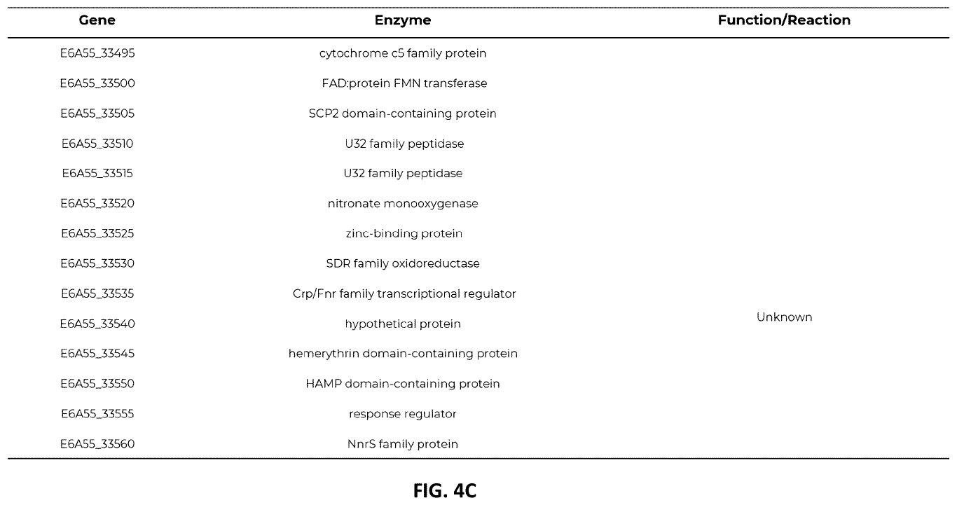

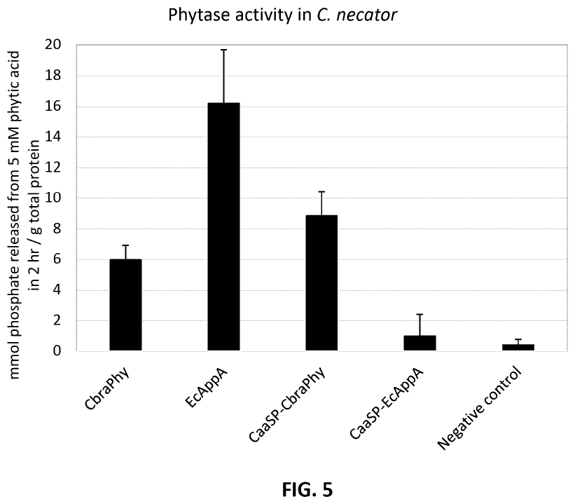

Without being bound by theory, specific examples of genetic modifications that may provide temperature resistance in the non-naturally occurring C1-fixing strain C. necator include, but are not limited to, mutations in a hydrogenase regulator (L405H (cTt→cAt) mutation in HoxA encoded by hoxA, E6Δ55_32285), deletions or frame shifts within certain regions (Δ35433 bp encompassing genes [E6Δ55_33395]-[E6Δ55_33560], the open reading frames of which are shown in FIGS. 4A-4C), or mutations in other genes associated with autotrophic growth. Thus, in some embodiments, a temperature resistant strain of C. necator may be genetically modified to possess a temperature resistant phenotype (as opposed to ALE).

Phenotypic features that the C. necator strain utilized for producing digestive enzymes may possess include, but are not limited to, a growth rate that is at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at temperatures above 30° C. For example the growth rate may be at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% higher than the growth rate of a wild type or naturally occurring strain of C. necator at 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C. Optionally, the improved growth at high temperatures may be observed when the C. necator is cultured in autotrophic conditions.

In some embodiments, the strain is Cupriavidus necator DSM 428, DSM 531, or DSM541, or any derivatives thereof. In another embodiment, the strain is Cupriavidus necator DSM 34774. In some embodiments, the strain comprises SEQ ID NOs: 1-3. In some embodiments, the strain is a derivative of Cupriavidus necator DSM 34774. In another embodiment, the strain is a derivative of SEQ ID NOs: 1-3.

Additionally or alternatively, the strain of C. necator utilized for the present disclosure may be modified to possess one or more other desirable traits. For example, the strain of C. necator may be modified either via genetic modification or ALE to minimize the H₂:CO₂ uptake ratio while maximizing intracellular O₂ during autotrophic growth. This can be done through the removal or reduction in a membrane-bound hydrogenase system. This can aid in reducing the cost of hydrogen associated with commercial-scale gas fermentation.

A strain of C. necator can be modified to minimize the H₂:CO₂ uptake ratio by deleting all or a portion of, or otherwise reducing the expression of a membrane-bound hydrogenase system (e.g., the hoxKGZ locus). The hoxKGZ locus encodes a multi-subunit membrane-bound hydrogenase system in Cupriavidus necator, and deleting this locus does not inhibit C. necator autotrophic growth on a gas mixture composed of hydrogen, oxygen, and carbon dioxide. Without being bound by theory, this may result because while the soluble hydrogenase system is required for autotrophic growth, the membrane-bound hydrogenase system wastefully oxidizes hydrogen gas, increasing the culture's H₂:CO₂ uptake ratio without benefiting growth. By deleting or reducing expression of the hoxKGZ locus, growth is not only permitted autotrophically, but the H₂:CO₂ uptake ratio is also reduced while increasing the intracellular O₂ availability to improve O₂-dependent product synthesis.

Phenotypic features that the C. necator strain utilized for producing digestive enzymes may possess include, but are not limited to, a H₂:CO₂ uptake ratio that is at least 5%, at least 10%, at least 15%, at least 20%, at least 25%, at least 30%, at least 35%, at least 40%, at least 45%, at least 50%, at least 55%, as at least 60%, at least 65%, at least 70%, at least 75%, at least 80%, at least 85%, at least 90%, at least 95%, or at least 100% lower than the H₂:CO₂ uptake ratio of a wild type or naturally occurring strain of C. necator cultured under corresponding temperatures and conditions. For example the H₂:CO₂ uptake ratio may be about 5%, about 10%, about 15%, about 20%, about 25%, about 30%, about 35%, about 40%, about 45%, about 50%, about 55%, as about 60%, about 65%, about 70%, about 75%, about 80%, about 85%, about 90%, about 95%, or about 100% lower than the H₂:CO₂ uptake ratio of a wild type or naturally occurring strain of C. necator cultured under corresponding temperatures and conditions. Optionally, the lower H₂:CO₂ uptake ratio may be observed at elevated culture temperatures, such as 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C. Optionally, the improved growth at elevated culture temperatures may be observed when the C. necator is cultured in autotrophic conditions.

For the purposes of the present disclosure, a strain of C. necator utilized to produce digestive enzymes may possess both temperature resistance and a reduced H₂:CO₂ uptake ratio relative to a wildtype strain of C. necator. For example, the strain deposited under accession number DSM 34774, or a strain sharing substantial phenotypic characteristics (e.g., temperature resistance) or genotypic characteristics (e.g., a genome with at least 80% sequence identity to SEQ ID NOs: 1-3 or a genome with at least 80% sequence similarity to SEQ ID NOs: 1-3) with a strain deposited under accession number DSM 34774 may be further modified by deleting all or a portion of, or otherwise reducing the expression of a membrane-bound hydrogenase system (e.g., the hoxKGZ locus), thus providing a strain of C. necator with both desirable features.

### V. Adaptive Laboratory Evolution (ALE)

Bacterial strains according to the disclosure can be evolved using adaptive laboratory evolution methods to improve heat tolerance and improve growth and biosynthesis in autotrophic conditions. However, it is understood by those skilled in the art and for the purposes of the present disclosure that ALE is not the only methodology that can be used to prepare a temperature resistant strain of C. necator, but merely one example of a suitable method.

ALE refers to the culture of cells or organisms under defined conditions leading to adaptive changes that accumulate in populations of cells or (microbial) organisms during selection under specified growth conditions. In particular, the desired trait is selected in an evolution environment where it provides a fitness benefit. In the target environment, the desired trait is exploited (for instance, heat resistance at elevated temperature conditions). In the target environment, the desired trait allows the increase or decrease of at least one desired trait and/or avoids the increase or decrease of at least one trait. In one specific embodiment, in the target environment the desired trait allows the increase in the production flux of a least one desired product or survival advantage.

Typically, a metabolic trait or a phenotypic trait of interest evolves over several generations of the cell or organism. This may include at least two generations, e.g., at least 10, at least 50, at least 100, at least 200, at least 300 or more generations, preferably at least 50 generations, more preferably about 100 generations or more of the cell or organism are necessary to evolve a metabolic trait. Thus, the cells or organisms are cultured for a certain time in a desired environment for, such as several days, weeks, months or years.

The target environment should be suitable to evolve a desired trait. When the population of cells or organisms is cultured in the target environment, a part of the population of the cell or organism will establish the desired trait, while some of the cells or organisms will die. In this way, the desired trait is selected. Preferably, at least 5%, at least 10%, at least 20%, at least 30%, at least 40%, at least 50%, at least 60%, at least 70%, at least 80%, at least 90% or more of the population have established the desired metabolic or phenotypic trait.

For the purposes of the present application, continuous ALE can be used to obtain a heat-resistant strain of C. necator capable of growth and culture at elevated temperatures, optionally in autotrophic conditions. As one example of continuous ALE, Example 1 of this disclosure describes inoculating a continuous-flow stirred tank reactor (CSTR) using a base strain of C. necator (H16 PHB-4). The culture can be continuously grown on a minimal autotrophic media with H₂/O₂/CO₂ inputs. Once the culture is established and growth was maintained at a steady state, the reactor temperature was increased step-wise and held to allow the culture to adapt to the increased temperature. These step wise changes may be continued over the course of several days; for example, 1-3 days, 3-50 days, 50-100 days, 50 days to 1 year, or 1-5 years.

The pH can be kept between pH 4 and 12, preferably between pH 6 and 9, particularly preferably between pH 7 and 8.

For the purposes of the present disclosure, ALE can be operated batchwise, semi-batchwise or continuously.

### V. Microbial Cultures

For the purposes of the present disclosure, C. necator may be grown under any suitable conditions, in an environment that is suitable for growth and production of biomass. For example, the C. necator may be grown in autotrophic culture conditions, heterotrophic culture conditions, or a combination of autotrophic and heterotrophic culture conditions.

A heterotrophic culture may include a suitable source of carbon and energy, such as one or more sugar (e.g., glucose, fructose, sucrose, etc.). An autotrophic culture may include C1 chemicals such as carbon monoxide, carbon dioxide, methane, methanol, formate, and/or formic acid, and/or mixtures containing C1 chemicals, including, but not limited to, various syngas compositions or various producer gas compositions, e.g., generated from low value sources of carbon and energy, such as, but not limited to, lignocellulosic energy crops, crop residues, bagasse, saw dust, forestry residue, or food, through the gasification, partial oxidation, pyrolysis, or steam reforming of said low value carbon sources, that can be used by an oxyhydrogen microorganism or hydrogen-oxidizing microorganism or carbon monoxide oxidizing microorganism as a carbon source and an energy source.

The culture systems used to grow C. necator described herein can be housed in culture vessels known and used in the art. In some embodiments, large scale production in a bioreactor vessel can be used to produce large quantities of a desired molecule and/or biomass. The bioreactor may include a fermentation tank for culturing the microorganisms under specific conditions that promote bacterial culture and growth.

Bioreactor vessels may be used to contain, isolate, and/or protect the culture environment. The culture vessels include those that are known to those of ordinary skill in the art of large scale microbial culturing. Such culture vessels include but are not limited to one or more of the following: airlift reactors; biological scrubber columns; bubble columns; stirred tank reactors; continuous stirred tank reactors; counter-current, upflow, expanded-bed reactors; digesters and in particular digester systems, for example, such known in the art of bioremediation; filters including but not limited to trickling filters, rotating biological contactor filters, rotating discs, soil filters; fluidized bed reactors; gas lift fermenters; immobilized cell reactors; loop reactors; membrane biofilm reactors; pachuca tanks; packed-bed reactors; plug-flow reactors; static mixers; trickle bed reactors; and/or vertical shaft bioreactors.

Microbial culturing may include the commercial production of biomass and/or organic compounds, e.g., protein product as described herein, specifically single cell protein, cell lysate, protein extract, protein-containing extract, protein concentrate, protein isolate, protein hydrolysate, free amino acids, peptides, oligopeptides, fatty acids or combinations thereof, and/or other nutrients, such as, but not limited to vitamins may be performed in bioreactors at large scale (e.g., 500 L, 1,000 L 5,000 L, 10,000 L, 50,000 L, 100,000 L, 1,000,000 L bioreactor volumes and higher).

The C. necator may be grown in a liquid media inside a bioreactor using methods described herein. The bioreactor containing the microorganisms may be constructed of opaque materials that keep the culture in near or total darkness. Bioreactors constructed out of opaque materials such as steel and/or other metallic alloys and/or reinforced concrete and/or fiberglass and/or various high strength plastic materials can be designed to have large working volumes. In some aspects, fermenters constructed of steel or other metallic alloys that are 50,000 liters and greater in volume may be utilized. In some aspects, bioreactors capable of containing positive headspace pressures above ambient pressure may be utilized. In some aspects, the bioreactor comprising the microorganism does not allow light to penetrate part or most or all of its contained liquid volume. The C. necator may be cultured without significant or any exposure to light. In certain such embodiments, net CO₂ consumption still occurs in the absence of light due to chemoautotrophic metabolism and conditions.

In some embodiments, the C. necator are grown and maintained in a medium containing a gaseous carbon source, such as but not limited to syngas, producer gas, or gas mixtures containing H₂ and CO₂, in the absence of light; where such growth is known as chemoautotrophic growth.

In some aspects, food grade CO₂ and/or air that goes through a direct air capture unit may be utilized by the microorganisms for chemoautotrophic growth. CO₂ may be provided from an industrial source, and optionally may be concentrated via a gas separation procedure, thereby resulting in high concentration food grade CO₂.

The bioreactor or fermenter may be used to culture cells through the various phases of their physiological cycle. A bioreactor is utilized for the cultivation of cells, which may be maintained at particular phases in their growth curve. The use of bioreactors is advantageous in many ways for cultivating chemoautotrophic growth. For certain embodiments, protein-rich cell mass, which is used to produce proteins or protein hydrolysates, is grown to high densities in liquid suspension. Generally, the control of growth conditions, including control of dissolved carbon dioxide, oxygen, and other gases such as hydrogen, as well as other dissolved nutrients, trace elements, temperature and pH, is facilitated in a bioreactor. For certain embodiments, protein-rich cell mass, which is used to produce amino acids, peptides, proteins, fatty acids, hydrolysates, extracts, or whole cell products, is grown to high densities and/or grown at high productivities, in liquid suspension within a bioreactor.

Nutrient media, as well as gases, can be added to the bioreactor as either a batch addition, or periodically, or in response to a detected depletion or programmed set point, or continuously over the period the culture is grown and/or maintained. The bioreactor at inoculation may be filled with a starting batch of nutrient media and/or one or more gases at the beginning of growth, and no additional nutrient media and/or one or more gases are added after inoculation. Nutrient media and/or one or more gases may be added periodically after inoculation. For certain embodiments, nutrient media and/or one or more gases are added after inoculation in response to a detected depletion of nutrient and/or gas. Nutrient media and/or one or more gases may be added continuously after inoculation. In some aspects, the added nutrient media does not contain any organic compounds.

In batch culture systems, the conditions (e.g., nutrient concentration, pH, etc.) under which the microorganism is cultivated generally change continuously throughout the period of growth. To avoid the fluctuating conditions inherent in batch cultures, and to improve the overall productivity of the culture system, the microorganisms that are used for the production of protein and/or other nutrients may be grown in a continuous culture system, such as a chemostat or a continuous-flow stirred tank reactor (CSTR). In such systems, the culture may be maintained in a perpetual exponential phase of growth by feeding it with fresh medium at a constant rate while at the same time maintaining the volume of the culture constant. A continuous culture system ensures that cells are cultivated under environmental conditions that remain roughly constant. Cells may be maintained in a perpetual exponential phase through the use of a chemostat system. In certain cases, the culture may be maintained in a steady state with a roughly fixed amount of standing biomass maintained in the bioreactor over time. The growth rate of a microorganism in continuous culture may be changed by altering the dilution rate. The growth rate of the microorganism may be changed by altering the dilution rate. In aspects, the continuous bioreactor may be maintained as a turbidostat, where a fixed amount of standing biomass is maintained in the bioreactor over time, and where all surplus biomass that is produced beyond that necessary to maintain the fixed amount of standing biomass within the bioreactor, is harvested continuously from the bioreactor.

Inoculation of the culture into the bioreactor may be performed by methods including, but not limited to, transfer of a C. necator culture from an existing culture inhabiting another bioreactor, or incubation from a seed stock raised in an incubator. The stock of the strain may be transported and stored in forms including but not limited to a powder, liquid, frozen, or freeze-dried form as well as any other suitable form, which may be readily recognized by one skilled in the art. Reserve bacterial cultures may be kept in a metabolically inactive, freeze-dried state until required for restart. When establishing a culture in a very large reactor, cultures may be grown and established in progressively larger intermediate scale vessels prior to inoculation of the full-scale vessel.

Bioreactors may have mechanisms to enable mixing of the nutrient media that include, but are not limited to, one or more of the following: spinning stir bars, blades, impellers, or turbines; spinning, rocking, or turning vessels; gas lifts, sparging; recirculation of broth from the bottom of the container to the top via a recirculation conduit, flowing the broth through a loop and/or static mixers. The culture media may be mixed continuously or intermittently.

The microorganism-containing nutrient medium may be removed from the bioreactor partially or completely, periodically or continuously. The microorganism-containing nutrient medium may be replaced periodically with fresh cell-free medium to maintain the cell culture in an exponential growth phase, and/or in another targeted growth phase (e.g. arithmetic growth), and/or to replenish the depleted nutrients in the growth medium, and/or remove inhibitory waste products.

The ports that are standard in bioreactors may be utilized to deliver, or withdraw, gases, liquids, solids, and/or slurries, into and/or from the bioreactor vessel enclosing the microbes. Many bioreactors have multiple ports for different purposes (e.g., ports for media addition, gas addition, probes for pH and dissolved oxygen, and sampling), and a given port may be used for various purposes during the course of a culture process. As an example, a port might be used to add nutrient media to the bioreactor at one point in time, and at another time might be used for sampling. Preferably, the multiple uses of a sampling port can be performed without introducing contamination or invasive species into the growth environment. A valve or other actuator enabling control of the sample flow or continuous sampling can be provided to a sampling port. The bioreactors may be equipped with at least one port suitable for culture inoculation that can additionally serve other uses including the addition of media or gas. Bioreactor ports enable control of the gas composition and flow rate into the culture environment. For example, the ports can be used as gas inlets into the bioreactor through which gases are pumped.

Gases that may be pumped into a bioreactor include, but not are not limited to, one or more of the following: syngas, producer gas, hydrogen gas, CO, CO₂, O₂, air, air/CO₂ mixtures, natural gas, methane, ammonia, nitrogen, noble gases, such as argon, as well as other gases. In some embodiments the CO₂ pumped into the system may come from sources including, but not limited to: CO₂ from the gasification of organic matter; CO₂ from the calcination of limestone, CaCO₃, to produce quicklime, CaO; CO₂ from methane steam reforming, such as the CO₂ byproduct from ammonia, methanol, or hydrogen production; CO₂ from combustion, incineration, or flaring; CO₂ byproduct of anaerobic or aerobic fermentation of sugar; CO₂ byproduct of a methanotrophic bioprocess; geologically or geothermally produced or emitted CO₂; CO₂ removed from acid gas or natural gas.

One or more gases in addition to carbon dioxide, or in place of carbon dioxide as an alternative carbon source, may either be dissolved into solution and fed to the culture broth and/or dissolved directly into the culture broth, including but not limited to gaseous electron donors and/or carbon sources (e.g., hydrogen and/or CO and/or methane gas). Input gases may include other electron donors and/or electron acceptors and/or carbon sources and/or mineral nutrients such as, but not limited to, other gas constituents and impurities of syngas (e.g., hydrocarbons); ammonia; hydrogen sulfide; and/or other sour gases; and/or O₂; and/or mineral containing particulates and ash.

One or more gases may be dissolved into the culture broth, including but not limited to gaseous electron donors such as, but not limited to, one or more of the following: hydrogen, carbon monoxide, methane, hydrogen sulfide or other sour gases; gaseous carbon sources such as, but not limited to one or more of the following: CO₂, CO, CH₄; and electron acceptors such as, but not limited to, oxygen, either within air (e.g., 20.9% oxygen) or as pure O₂ or as an O₂-enriched gas. The dissolution of these and other gases into solution may be achieved using a system of compressors, flowmeters, and flow valves known to one skilled in the art of fermentation engineering, that feed into one of more of the following widely used systems for dispersing gas into solution: sparging equipment; diffusers including but not limited to dome, tubular, disc, or doughnut geometries; coarse or fine bubble aerators; venturi equipment. Surface aeration and/or gas mass transfer may also be performed using paddle aerators and the like. In certain aspects, gas dissolution may be enhanced by mechanical mixing with an impeller or turbine, as well as hydraulic shear devices to reduce bubble size.

Following passage through the reactor system holding microorganisms which uptake the gases, in certain embodiments the residual gases may either be recirculated back to the bioreactor, or burned for process heat, or flared, or injected underground, or released into the atmosphere.

In some aspects, the C. necator may grow on H₂ and CO₂ and other dissolved nutrients under microaerobic conditions. In some aspects, a C1 chemical such as but not limited to carbon monoxide, methane, methanol, formate, or formic acid, and/or mixtures containing C1 chemicals including but not limited to various syngas compositions generated from various gasified, pyrolyzed, or steam-reformed fixed carbon feedstocks, are biochemically converted into longer chain organic chemicals (i.e., C2 or longer and, in some embodiments, C5 or longer carbon chain molecules) under one or more of the following conditions: aerobic, microaerobic, anoxic, anaerobic, and/or facultative conditions.

A controlled amount of oxygen can also be maintained in the culture broth of some.

Oxygen may be actively dissolved into solution fed to the culture broth and/or directly dissolved into the culture broth. In some aspects, conditions suitable for growth of an oxyhydrogen microorganism may be deployed, such as use of H₂ and O₂ gas substrates (electron donors and acceptors), and optionally a C1 gaseous carbon source, such as CO₂ and/or CO.

The C. necator may convert a fuel gas, including but not limited to syngas, producer gas, CO, CO₂, H₂, natural gas, methane, and mixtures thereof. In some embodiments, the heat content of the fuel gas is at least 100 BTU per standard cubic foot (scf). In some embodiments, a bioreactor that is used to contain and grow the microorganisms is equipped with fine-bubble diffusers and/or high-shear impellers for gas delivery.

Introducing and/or raising the gas flow rate into a bioreactor can enhance mixing of the culture and produce turbulence if the gas inlet is positioned beneath the surface of the liquid media such that gas bubbles or sparges up through the media. Mixing may be enhanced through turbulence provided by gas bubbles and/or sparging and/or gas plugging up through the liquid media. The bioreactor may include gas outlet ports for gas escape and pressure release. Gas inlets and outlets may be equipped with check valves to prevent gas backflow.

A nutrient media for culture growth and production may be used, including an aqueous solution containing suitable minerals, salts, vitamins, cofactors, buffers, and other components needed for microbial growth, known to those skilled in the art [Bailey and Ollis, Biochemical Engineering Fundamentals, 2nd ed; pp 383-384 and 620-622; McGraw-Hill: New York (1986)].

Chemicals used for maintenance and growth of microbial cultures as known in the art are included in the nutrient media. These chemicals may include but are not limited to one or more of the following: nitrogen sources such as ammonia, ammonium (e.g., ammonium chloride (NH₄Cl), ammonium sulfate ((NH₄)₂SO₄)), nitrate (e.g., potassium nitrate (KNO₃)), urea or an organic nitrogen source; phosphate (e.g., disodium phosphate (Na₂HPO₄), potassium phosphate (KH₂PO₄), phosphoric acid (H₃PO₄), potassium dithiophosphate (K₃PS₂O₂), potassium orthophosphate (K₃PO₄), dipotassium phosphate (K₂HPO₄)); sulfate; yeast extract; chelated iron; potassium (e.g., potassium phosphate (KH₂PO₄), potassium nitrate (KNO₃), potassium iodide (KI), potassium bromide (KBr)); and other inorganic salts, minerals, and trace nutrients (e.g., sodium chloride (NaCl), magnesium sulfate (MgSO₄7H₂O) or magnesium chloride (MgCl₂), calcium chloride (CaCl₂) or calcium carbonate (CaCO₃), manganese sulfate (MnSO₄7H₂O) or manganese chloride (MnCl₂), ferric chloride (FeCl₃), ferrous sulfate (FeSO₄7H₂O) or ferrous chloride (FeCl₂ 4H₂O), sodium bicarbonate (NaHCO₃) or sodium carbonate (Na₂CO₃), zinc sulfate (ZnSO₄) or zinc chloride (ZnCl₂), ammonium molybdate (NH₄MoO₄) or sodium molybdate (Na₂MoO₄2H₂O), cuprous sulfate (CuSO₄) or copper chloride (CuCl₂ 2H₂O), cobalt chloride (CoCl₂ 6H₂O), aluminum chloride (AlCl₃.6H₂O), lithium chloride (LiCI), boric acid (H₃BO₃), nickel chloride NiCl₂6H₂O), tin chloride (SnCl₂ H₂O), barium chloride (BaCl₂ 2H₂O), copper selenate (CuSeO₄5H₂O) or sodium selenite (Na₂SeO₃), sodium metavanadate (NaVO₃), chromium salts).

C. necator strains described herein can be cultured in media of any type (rich or minimal), including fermentation medium, and any composition. The selected medium can be supplemented with various additional components. Some non-limiting examples of supplemental components include glucose, fructose, sucrose, starches, polysaccharides, protein hydrolysates, antibiotics, IPTG for gene induction, and ATCC Trace Mineral Supplement. Similarly, other aspects of the medium and growth conditions described herein may be optimized through routine experimentation. For example, pH and temperature are non-limiting examples of factors which can be optimized. In some embodiments, factors such as choice of media, media supplements, and temperature can influence production levels of a desired molecule. In some embodiments, the concentration and amount of a supplemental component may be optimized. In some embodiments, how often the media is supplemented with one or more supplemental components, and the amount of time that the media is cultured before harvesting the desired molecule is optimized.

The concentrations of nutrient chemicals (e.g., carbon sources, and/or various mineral nutrients), may be maintained within the bioreactor close to or at their respective optimal levels for optimal carbon uptake and/or fixation and/or conversion and/or production of biomass and/or organic compounds, and in particular protein, which may be routinely determined and/or optimized by one of ordinary skill in the art of culturing microorganisms.

One or more of the following parameters may be monitored and/or controlled in the bioreactor: waste product levels; pH; temperature; salinity; dissolved oxygen; dissolved carbon dioxide gas; liquid flow rates; agitation rate; gas pressure. In certain embodiments, the operating parameters affecting chemoautotrophic growth, and/or other types of growth (e.g., heterotrophic growth) are monitored with sensors (e.g., dissolved oxygen probe or oxidation-reduction probe to gauge electron donor/acceptor concentrations), and/or are controlled either manually or automatically based upon feedback from sensors through the use of equipment including but not limited to actuating valves, pumps, and agitators. The temperature of the incoming broth as well as of incoming gases may be regulated by systems such as, but not limited to, coolers, heaters, and/or heat exchangers.

The C. necator culture and bioreaction may be maintained using continuous influx and removal of nutrient medium and/or biomass, in steady state where the cell population and environmental parameters (e.g., cell density, pH, DO, chemical concentrations) are targeted at a constant level over time.

In some aspects, the pH of the microbial culture is controlled. pH may be controlled within an optimal range for microbial maintenance and/or growth and/or conversion of feedstock and/or production of organic compounds and/or survival. To address a decrease in pH, a neutralization step can be performed directly in the bioreactor environment or prior to recycling the media back into the culture vessel through a recirculation loop. Neutralization of acid in the broth of certain embodiments can be accomplished by the addition of bases, including but not limited to one or more of the following: limestone, lime, sodium hydroxide, ammonia, ammonium hydroxide, caustic potash, magnesium oxide, iron oxide, alkaline ash.

The culture systems may be carried out at varying temperatures controlled by the bioreactor system. The temperature of the culture system may be controlled to optimize enzyme function, protein biosynthesis, or biomass production. Additionally or alternatively, the temperature of the culture system may be dictated by the incorporation of the culture system into an industrial process with a temperature above 30° C. In such situations, the temperature of the culture system may be 31° C., 32° C., 33° C., 34° C., 35° C., 36° C., 37° C., 38° C., 39° C., or 40° C. or within a range of 32-40° C., 33-40° C., 34-40° C., 35-40° C., 31-39° C., 32-39° C., 33-39° C., 34-39° C., or 35-39° C.

VI. Engineered Strains of C. necator For Producing Digestive Enzymes

While C. necator provides an excellent bacterial strain for numerous applications, in many single cell protein (SCP) applications, such as feed products, digestive enzymes are added to the final product in aid in digestibility. Enzymes that facilitate digestion by breaking down starch, protein, and fat in the digestive system or increasing the availability of nutrients can provide significant nutritional value to feed products. Through the development of engineered C. necator strains that heterologously express stable digestive enzymes, such as proteases, carbohydrases, lipases, and/or phytases, among others, specific single cell protein products can be generated that both enhance the nutritional properties of SCP and aid in unlocking the full nutritional value of any final feed product in which SCP serves as a major constituent. As provided in more detail below, the engineered strains of C. necator provided herein are capable of producing digestive enzymes.

The engineered C. necator strains of the present disclosure express at least one transgene encoding a heterologous digestive enzymes, wherein the strain of C. necator synthesizes at least one digestive enzyme that is not natively produced by C. necator. The digestive enzyme may be selected from a protease, a carbohydrase, a lipase, a phytase, and any combination thereof. The present disclosure also provides strains of C. necator that expresses a heterologous digestive enzyme encoded by a transgene, wherein the strain of C. necator synthesizes an increased amount of digestive enzyme relative to a strain of C. necator that does not express the heterologous digestive enzyme. Transgenes encoding various digestive enzymes which are advantageously used in the process according to the invention are those which encode polypeptides with protease, carbohydrase, lipase, and/or phytase activity.

The engineered strains of C. necator are also able to synthesize an increased amount of a digestive enzyme relative to a strain of C. necator that does not express the heterologous digestive enzyme. The digestive enzymes may be a protease, a carbohydrase, a lipase, and/or a phytase.

### A. Digestive Enzymes

Transgenes encoding various digestive enzymes that may be incorporated into and/or expressed by the engineered strains of C. necator include, but are not limited to, transgenes that encode a protease, a carbohydrase, a lipase, and/or a phytase enzyme. In other words, the heterologous digestive enzymes that are expressed by the engineered strains of C. necator include, but are not limited to, one or more of a protease, a carbohydrase, a lipase, a phytase or any combination thereof.

The nucleic acid sequences that encode proteins with protease, carbohydrase, lipase, and/or phytase activity can be codon optimized for expression in C. necator regardless of the native species from which they are derived. The nucleic acids can be transfected into the disclosed engineered C. necator alone or in combination, for example, in an expression cassette (i.e., a nucleic acid construct) that the allows expression of the nucleic acids in the C. necator. There can be more than one nucleic acid sequence encoding a protein with a desired enzymatic activity in the nucleic acid construct, such as, for example, a protease, a carbohydrase, a lipase, and/or a phytase may be included. In some embodiments, a protease, a carbohydrase, a lipase, and/or a phytase may be co-expressed.

The copy number of the heterologous protease, carbohydrase, lipase, and/or phytase genes can be increased, so that the level of protein expression is increased and the yield, production and/or production efficiency of the desired digestive enzymes is also increased.

The strategy of increasing copy number of the transgene or expression level of the heterologous protein applies analogously to the combination with further digestive enzymes involved in fatty acid, lipid, carbohydrate, or protein metabolism.

It is, however, to be understood that the engineered strains of C. necator disclosed herein are not limited to expression of protease, carbohydrase, lipase, and/or phytase from Bacillus (Bacillus), Clostridium (Clostridium), Enterococcus (Enterococcus), Geobacillus (Geobacillus), Lactobacillus (Lactobacillus), Lactococcus (Lactococcus), marine Bacillus (Oceanobacillus), Staphylococcus (Staphylococcus), Streptococcus (Streptococcus), or Streptomyces (Streptomyces) protease; or a gram-negative bacterial polypeptide, such as Campylobacter (Campylobacter), Escherichia coli (E. coli), Flavobacterium (Flavobacterium), Clostridium (Fusobacterium), Helicobacter (Helicobacter), Clavibacterium (Ilyobacter), Neisseria (Neisseria), Pseudomonas (Pseudomonas), Salmonella (Salmonella) or Ureabasma (Ureabasma), but rather may express any number or combination of suitable exogenous or heterologous digestive enzymes.

Heterologous digestive enzymes and the transgenes that express them can be derived from a variety of organisms that natively produce desirable digestive enzymes, such as certain bacteria, cyanobacteria, fungi, algae, and other microbes or animals.

Thus, heterologous digestive enzymes, as well as nucleic acid sequences encoded the same, can be derived from a bacteria such as a bacteria selected from Bacillus, Clostridium, Enterococcus, Geobacillus, Lactobacillus, Lactococcus, marine Bacillus, Staphylococcus, Streptococcus, Streptomyces Campylobacter, Escherichia coli, Flavobacterium, Helicobacter, Clavibacterium, Neisseria, Pseudomonas, Salmonella or Ureabasma. In some aspects, the digestive enzyme is derived from Bacillus species. In some aspects, Bacillus species includes Bacillus alkalophilus, Bacillus amyloliquefaciens, Bacillus brevis, Bacillus circulans, Bacillus clausii, Bacillus coagulans, Bacillus sp. D-6, Bacillus firmus, Bacillus sp. KSM-KP43, Bacillus lautus, Bacillus lentus, Bacillus licheniformis, Bacillus megaterium, Bacillus pumilus, Bacillus stearothermophilus, Bacillus subtilis, Bacillus thuringiensis.

Strains of these species are readily available to the public at a number of culture collections, such as the American Type Culture Collection (ATCC), the German Collection of microorganisms and cell cultures (Deutsche Sammlung von Mikroorganismen und Zellkulturen GmbH, DSMZ), the Netherlands collection of species (CBS), and the northern regional research center of the American Collection of agricultural research species (NRRL).

Thus, heterologous digestive enzymes, as well as nucleic acid sequences encoded the same, can be derived from a fungi such as a fungi selected from Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium acuminatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thermoactinomyces vulgaris, Thielavia terrestris, Trametes villosa, Trametes versicolor, Trichoderma harzianum, Trichoderma koningii, Trichoderma longibrachiatum, Trichoderma reesei, or Trichoderma viride.

### B. Protease Enzymes

C. necator does not naturally produce significant amounts of digestive enzymes exhibiting protease activity. In the context of a single cell protein product, for example, in the use of animal feed produces, may have a significant beneficial effect on the performance of an animal, including improving one or more of the following: feed conversion ratio (FCR), ability to digest a raw material (e.g. nutrient digestibility, such as amino acid digestibility), nitrogen retention, survival, carcass yield, growth rate, weight gain, feed efficiency animals resistance to necrotic enteritis, immune response of the subject, the growth of beneficial bacteria in the gastrointestinal tract of a subject.

Accordingly, the present disclosure provides for the introduction of nucleic acids encoding various proteases. Thus, the engineered C. necator of the present disclosure may be modified to express one or more protease enzymes, including, but not limited to, serine proteases trypsin, chymotrypsin, subtilisin, carboxypeptidase, elastase, nuclease, collagenase, dipeptidase, pepsin, rennin, thrombin, plasmin, renin, hyaluronidase, insulinase, chymase, tryptase, cathepsin, and neurolysin.

Suitable exogenous or heterologous nucleic acid sequences that encode a protein with serine protease activity include, but are not limited to, a nucleic acid sequences that code for proteins or polypeptides having serine protease activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Bacillus amyloliquefaciens BPN:

Bacillus amyloliquefaciens BPN:
AQSVPYGVSQIKAPALHSQGYTGSNVKVAVIDSGIDSSHPDLKVAGGASMVPSETNP
FQDNNSHGTHVAGTVAALNNSIGVLGVAPSASLYAVKVLGADGSGQYSWIINGIEW
AIANNMDVINMSLGGPSGSAALKAAVDKAVASGVVVVAAAGNEGTSGSSSTVGYP
GKYPSVIAVGAVDSSNQRASFSSVGPELDVMAPGVSIQSTLPGNKYGAYNGTSMASP
HVAGAAALILSKHPNWTNTQVRSSLENTTTKLGDSFYYGKGLINVQAAAQ
Bacillus alcalophilus Maxacal Mutant
AQSVPWGISRVQAPAAHNRGLTGSGVKVAVLDTGISTHPDLNIRGGASFVPGEPSTQ
DGNGHGTHVAGTIAALNNSIGVLGVAPNAELYAVKVLGASGSGSVSSIAQGLEWAG
NNGMHVANLSLGMAGPSATLEQAVNSATSRGVLVVAASGNSGAGSISYPARYANA
MAVGATDQNNNRASFSQYGAGLDIVAPGVNVQSTYPGSTYASLNGTSMATPHVAG
AAALVKQKNPSWSNVQIRNHLKNTATSLGSTNLYGSGLVNAEAATRGGGSWSHPQ
FEKGGGSGGGSGGSAWSHPQFEK
Bacillus sp. D-6 E1:
NDVARGIVKADVAQNNYGLYGQGQVVAVADTGLDTGRNDSSMHEAFRGKITALY
ALGRTNNANDPNGHGTHVAGSVLGNALNKGMAPQANLVFQSIMDSSGGLGGLPSN
LNTLFSQAWNAGARIHTNSWGAPVNGAYTANSRQVDEYVRNNDMTVLFAAGNEG
PNSGTISAPGTAKNAITVGATENYRPSFGSIADNPNHIAQFSSRGATRDGRIKPDVTAP
GTFILSARSSLAPDSSFWANYNSKYAYMGGTSMATPIVAGNVAQLREHFIKNRGITP
KPSLIKAALIAGATDVGLGYPSGDQGWGRVTLDKSLNVAYVNEATALTTGQKATYS
FQTQAGKPLKISLVWTDAPGSTTASYTLVNDLDLVITAPNGQKYVGNDFSYPYDNN
WDGRNNVENVFINAPQSGTYTIEVQAYNVPSGPQRFSLAIVHGGGSWSHPQFEKGG
GSGGGSGGSAWSHPQFEK
Bacillus lentus Savinase:
AQSVPWGISRVQAPAAHNRGLTGSGVKVAVLDTGISTHPDLNIRGGASFVPGEPSTQ
DGNGHGTHVAGTIAALNNSIGVLGVAPSAELYAVKVLGASGSGSVSSIAQGLEWAG
NNGMHVANLSLGSPSPSATLEQAVNSATSRGVLVVAASGNSGAGSISYPARYANAM
AVGATDQNNNRASFSQYGAGLDIVAPGVNVQSTYPGSTYASLNGTSMATPHVAGA
AALVKQKNPSWSNVQIRNHLKNTATSLGSTNLYGSGLVNAEAATR
Bacillus sp. KSM-KP43 KP-43:
NDVARGIVKADVAQSSYGLYGQGQIVAVADTGLDTGRNDSSMHEAFRGKITALYAL
GRTNNANDTNGHGTHVAGSVLGNGSTNKGMAPQANLVFQSIMDSGGGLGGLPSNL
QTLFSQAYSAGARIHTNSWGAAVNGAYTTDSRNVDDYVRKNDMTILFAAGNEGPN
GGTISAPGTAKNAITVGATENLRPSFGSYADNINHVAQFSSRGPTKDGRIKPDVMAPG
TFILSARSSLAPDSSFWANHDSKYAYMGGTSMATPIVAGNVAQLREHFVKNRGITPK
PSLLKAALIAGAADIGLGYPNGNQGWGRVTLDKSLNVAYVNESSSLSTSQKATYSFT
ATAGKPLKISLVWSDAPASTTASVTLVNDLDLVITAPNGTQYVGNDFTSPYNDNWD
GRNNVENVFINAPQSGTYTIEVQAYNVPVGPQTFSLAIVNGGGSWSHPQFEKGGGSG
GGSGGSAWSHPQFEK
Bacillus lichenformis Alcalase:
AQTVPYGIPLIKADKVQAQGFKGANVKVAVLDTGIQASHPDLNVVGGASFVAGEAY
NTDGNGHGTHVAGTVAALDNTTGVLGVAPSVSLYAVKVLNSSGSGSYSGIVSGIEW
ATTNGMDVINMSLGGASGSTAMKQAVDNAYAKGVVVVAAAGNSGSSGNTNTIGY
PAKYDSVIAVGAVDSNSNRASFSSVGAELEVMAPGAGVYSTYPTNTYATLNGTSMA
SPHVAGAAALILSKHPNLSASQVRNRLSSTATYLGSSFYYGKGLINVEAAAQ
Bacillus subtilis 168 subtilisin 168:
AGKSSTEKKYIVGFKQTMSAMSSAKKKDVISEKGGKVQKQFKYVNAAAATLDEKA
VKELKKDPSVAYVEEDHIAHEYAQSVPYGISQIKAPALHSQGYTGSNVKVAVIDSGI
DSSHPDLNVRGGASFVPSETNPYQDGSSHGTHVAGTIAALNNSIGVLGVAPSASLYA
VKVLDSTGSGQYSWIINGIEWAISNNMDVINMSLGGPTGSTALKTVVDKAVSSGIVV
AAAAGNEGSSGSTSTVGYPAKYPSTIAVGAVNSSNQRASFSSAGSELDVMAPGVSIQ
STLPGGTYGAYNGTSMATPHVAGAAALILSKHPTWTNAQVRDRLESTATYLGNSFY
YGKGLINVQAAAQ
Bacillus subtilis subtilisn:
AGKSNGEKKYIVGFKQTMSTMSAAKKKDVISEKGGKVQKQFKYVDAASATLNEKA
VKELKKDPSVAYVEEDHVAQAYAQSVPYGVSQIKAPALHSQGFTGSNVKVAVIDSG
IDSSHPDLKVAGGASMVPSETNPFQDNNSHGTHVAGTVAALNNSVFVLGVAPSASL
YAVKVLGADGSGQYSWIINGIEWAIANNMDVINMSLGGPSGSAALKAAVDKAVAS
GVVVVAAAGNEGTSGGSSTVGYPGKYPSVIAVGAVNSSNQRASFSSVGSELDVMAP
GVSIQSTLPGNKYGAYNGTSMASPHVAGAAALILFKHPNWTNTQVRSSLENTTTKL
GDAFYYGKGLINVQAAAH
Aspergillus clavatus AlpES1 A. clavatus:
APAREPHPSSNIIPGKYIITFKSGIDTAAIESHTAWASNIHKRNLERRGLVGGEFPAGIE
RKFKIKDFAAYAGSFDPATIEEIRNSEDVAHVEEDQIWYLDALTTQSGAPWGLGSISH
KGQASTNYVYDTSAGAGTYAYVVDSGINVDHIEFQGRATKAYNAVGGDHVDTLGH
GTHVAGTIGGKTYGVAKQTNLLSVKVFEGRTGSTSVILDGENWAANDIVSKGRKGK
AAINMSLGGGYSYAFNNAVESAYEQGVLSVVAAGNEGVDASNSSPASAPNALTVG
ATNKSNARASFSNYGKVLDIFAPGQDILSAWIGSTTATNTISGTSMATPHVVGLAVY
LMGLEGVSGPAAVTQRILQLATSGVISDVKGSPNKLAYNGAA
Streptomyces S8 protease:
AQPEGRVLHAQAAEAVPGSYIVTLKPTSGFKASATEGKQLIAGYGGRITRTYTSALN
GYAAALSSAEARRLAADPAVESVEQDQKVHATATQTNAPWGLDRIDQANLPLNGT
YTYPDSAGGGTTVYVLDTGVRITHQQISGRASYGYDFVDNDAVAQDGAGHGTHVA
TTVAGSTYGVAKKAKIVAVRVLDNNGSGTTAGVIAGVDWITANHVASSVANVSLG
GGPSTALDDAVRRSIASGVTYSIAAGNSGAPASGYSPARVDTAITVGATTRTDARAS
YSNYGPVVDIFAPGSDITAGWNTSDTATYTGSGTSFAAPHVAGAAAVYLTNHPGSSP
AAVASALVNGATSNVLTGIGTGSPNKLLKLVP
Cupriavidus necator H16 serine protease:
APLPDSNRAAPGTEAARSARLVDRDGDRISDDLQARIAAARPGDLVRVVVTFSGPGT
AATARQAVGAFTLDREFRIIRGFSATMTTAQIRALAQQPGVRRIEEDFTVSTKLNAAR
ADFGTDATRTSFGVDGAGIKGCIVDTGVDSNHEQLDARPIPFFDAINGRTSAYDDHG
HGTHVASIAFGDGTGGSGAATYKGVAPGAAIYAAKVLDQSGSGLESQVIAGIDWCV
SQGVRIISMSLGSSAPSDGQDSLSQAVDAAVSSGAVVVVAAGNSGDEPGTVGSPGAA
AQAITVAACAEWSAPAGAPNHSDGIYVTPFSSRGPTLDNRVKPDICAPGLTITAAKA
GTVSGYVTYSGTSMATPFVSGIVALALQANPILSPADLRQKLEVTAQDRGPAGKDND
IGAGLLDGYALVASAKGGTSYQPTVFPTYRRVTGSVANHGQWSYTFSIAQADLGVPI
GATVTINGQAKCILPLLGGCFAAQWDPDLDARLLDPNGVVLSESLCMAGVECGGIG
RQETLHALPTVAGTYTIQVSPSEDSVNLGKGGSFALDLSSGPRAGIASLPVVHVGGLS
ASSTGNKTGWTTTVTVTVHDASHNPVPNATVSGTWSGGYSGSSACTTGAGGQCMV
TSGTISKRKNSVTFAVTNITGTLTYQASSNDMTSVTATKP
Thermoactinomyces vulgaris thermitase:
YTPNDPYFSSRQYGPQKIQAPQAWDIAEGSGAKIAIVDTGVQSNHPDLAGKVVGGW
DFVDNDSTPQNGNGHGTHCAGIAAAVTNNSTGIAGTAPKASILAVRVLDNSGSGTW
TAVANGITYAADQGAKVISLSLGGTVGNSGLQQAVNYAWNKGSVVVAAAGNAGN
TAPNYPAYYSNAIAVASTDQNDNKSSFSTYGSVVDVAAPGSWIYSTYPTSTYASLSG
TSMATPHVAGVAGLLASQGRSASNIRAAIENTADKISGTGTYWAKGRVNAYKAVQ
Y
Bacillus subtilis DY Subtilisin DY BSSDY:
AQTVPYGIPLIKADKVQAQGYKGANVKVGIIDTGIAASHTDLKVVGGASFVSGESYN
TDGNGHGTHVAGTVAALDNTTGVLGVAPNVSLYAIKVLNSSGSGTYSAIVSGIEWA
TQNGLDVINMSLGGPSGSTALKQAVDKAYASGIVVVAAAGNSGSSGSQNTIGYPAK
YDSVIAVGAVDSNKNRASFSSVGAELEVMAPGVSVYSTYPSNTYTSLNGTSMASPH
VAGAAALILSKYPTLSASQVRNRLSSTATNLGDSFYYGKGLINVEAAAQ
Fusarium acuminatum (CBS 124084) serine protease:
ALTTQSGAPWGLGAISHKSSGSTSYIYDTTAGSGSYGYVVDSGINIAHTDFGGRATLG
YNAAGGAHTDTLGHGTHVAGTIGGTKYGVSKKANLISVKVFAGNQAATSVILDGEN
WAVNDITSKGRAGKSVINMSLGGPSSATWTTAINAGYNAGVLSVVAAGNGDVNGN
PLPVSSQSPANAPNALTVAAIDSNWRTASFTNYGAGVDIFGPGVNILSAWIGSSTATN
TISGTSMASPHLAGLALYLQVLEGLSTPAAVTNRIKALGTSGKVTGSLSGSPNLVAY
NGNG

Suitable exogenous or heterologous nucleic acid sequences encoding a protein with serine protease activity may also include nucleic acid sequences which can be derived as a result of the degenerate genetic code from the amino acid sequences identified above as well as functional equivalents with serine protease activity.

Suitable exogenous or heterologous nucleic acid sequences that that encode a protein with trypsin activity, chymotrypsin activity, carboxypeptidase activity, elastase activity, nuclease activity, collagenase activity, dipeptidase activity, pepsin activity, dipeptidase activity, rennin activity, thrombin activity, plasmin activity, hyaluronidase activity, insulinase activity, chymase activity, tryptase activity, cathepsin activity, or neurolysin activity.

For the purposes of the present disclosure, heterologous proteases and the transgenes that express them can be derived from a variety of organisms that natively express such enzymes, such as certain bacteria, cyanobacteria, fungi, algae, and other microbes or animals. Thus, heterologous proteases, as well as nucleic acid sequences encoding the same, can be derived from Bacillus species (e.g., Bacillus amyloliquefaciens or Bacillus lentus), Aspergillus species, Streptomyces species, Cupriavidus species, Thermoactinomyces species, and Fusarium species.

In certain aspects, the C. necator may be engineered to express one or more functional fragments of a protease enzyme. The phrase “functional fragment” as used herein refers to a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length polypeptide. The functional fragment can generally, but not always, be comprised of a continuous region of the protein, wherein the region has functioning protease activity.

An engineered strain of C. necator can express a protease alone or in combination with one or more of digestive enzyme, including but not limited to, other protease enzymes, a carbohydrase, a lipase, and/or a phytase.

### C. Carbohydrase Enzymes

C. necator does not naturally produce significant amounts of digestive enzymes exhibiting carbohydrase activity. Accordingly, the present disclosure provides for the introduction of nucleic acids encoding various carbohydrases. Thus, the engineered C. necator of the present disclosure may be modified to express one or more carbohydrase enzymes, including, but not limited to, glucosidases, glucanases, xylanases, galactosidases, fructofuranosidases, mannosidases, thioglucosidases, amylases, maltases, lactases, sucrases, cellulases, hexosanases, pentosanases, fructosyltransferases, chitinases, and/or pectinases. By improving long chain carbohydrase enzyme synthesis in C. necator relative to a wild type strain, the resulting engineered bacterium is capable of producing higher levels various carbohydrase that may aid in digestion and metabolism. Accordingly, various carbohydrase enzymes may be used in combination with the above-disclosed proteases to provide a further engineered biosynthesis of digestive enzymes.

Carbohydrase enzymes that may be expressed in the engineered C. necator of the present disclosure include, but are not limited to, glucosidases, glucanases, xylanases, galactosidases, fructofuranosidases, mannosidases, thioglucosidases, amylases, maltases, lactases, sucrases, cellulases, hexosanases, pentosanases, fructosyltransferases, chitinases, and pectinases.

Suitable exogenous or heterologous nucleic acid sequences that that encode a protein with mannanase activity include, but are not limited to, a nucleic acid sequences that code for proteins or polypeptides having mannanase activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Mannan endo-1,4-beta-mannosidase (EC 3.2.1.78) (1,4-beta-D-mannan mannanohydrolase) (Beta-mannanase) (Glucomannan utilization protein G) Bacillus subtilis (strain 168)

MFKKHTISLLIIFLLASAVLAKPIEAHTVSPVNPNAQQTTKTVMNWLAHLPNRTENR
VLSGAFGGYSHDTFSMAEADRIRSATGQSPAIYGCDYARGWLETANIEDSIDVSCNG
DLMSYWKNGGIPQISLHLANPAFQSGHFKTPITNDQYKKILDSSTVEGKRLNAMLSKI
ADGLQELENQGVPVLFRPLHEMNGEWFWWGLTSYNQKDNERISLYKQLYKKIYHY
MTDTRGLDHLIWVYSPDANRDFKTDFYPGASYVDIVGLDAYFQDAYSINGYDQLTA
LNKPFAFTEVGPQTANGSFDYSLFINAIKQKYPKTIYFLAWNDEWSAAVNKGASALY
HDSWTLNKGEIWNGDSLTPIVE

Mannan endo-1,4-beta-mannosidase (EC 3.2.1.78) (Beta-mannanase) (Mannanase 26A) (Man26A) (Mannanase A) (ManA) Bifidobacterium adolescentis (strain ATCC 15703/DSM 20083/NCTC 11814/E194a)

MKTTVTKLLATVAAASTIFGMSTLPAFAAEGKSASNGNSVNISDVNATAETRALFDK
LKNSGKGDLRFGQQHATDENISSSASQGDVYETTGKYPAVFGWDAGLALRGAEKPG
SGADKNANAKALAQNITDADSKGAIVTLSAHWCNPGTGKDENDTTAVASELLPGGK
YSGTFNKELDAIAATAQRAKRSDGTLIPIIFRPLHENNGSWFWWGATHASASEYKEL
YRYIVDYLRDVKDVHNLLYAYSPGGVENGDSTDYLATYPGDQWVDVLGYDEYDSD
DSADDSSAWINTVVKDMKMVSDQASQRGKIVALTEFGRSGDRKFKESGTGDKDTK
FFSELAEALAENVPSTAYMMTWANFGGGGDNFQAYTSWKGSDGEADFKAFADSNK
NLMASKDNVDYSNAPAAAMQNGSARIVTPVDGNRVTDTKVVVRVKTEGVKYSDL
DLNSAIVTTDRGQNVKLKYSCNGYFTGILDLNAAGINLDQSKLTLTPQVKTKDGKTL
AAADGNGSVTVKLGAKPEQTVDNVEDFDSYDNEAELQSVYSPSHSTKSNLTLVDSP
EDNGTKAGNIHYDFVSYPEYNGFQRSHTPKQDWSGFSKLNMFLKADGSDHKFVVQ
VNAGGVTFEAYPKIDGTDGHVVSLNFGDADGNGGDFAPASWDTAHAGMKLSQKLL
SKVGSFALYINDNGGNRPKSGDLTLDSIKLDGKRDAYAPNTNPTPGNTAKAQSVDDF
SGYSDDAAAQSAWGNRGHTEVLSLDEGPTDGSKALRFKYDFSNGGWYDVAKYLD
GANWSGESVLAFQVKGDGSGNAIGLQIGTSDGKYFLASVKLDFTGWKQIEIPLVDNA
NLTQSWPEDANKDNPMTEDDLASIKELVFASQQWNSESDGLDSSIADIKVEPAENTS
NEQTPKDESKTEVKADKEQEQSEDTSADVTAQDPATCPISDEDSKGSTGNTTVTVKP
TPDTKEPADNTGKDGLSRTGSNIISAIAAVAVLLLGGCAVLIARKRKGGDIE

Mannan endo-1,4-beta-mannosidase A and B (EC 3.2.1.78) (1,4-beta-D-mannan mannanohydrolase) (Beta-mannanase) (Endo-1,4-mannanase) [Cleaved into: Mannan endo-1,4-beta-mannosidase A; Mannan endo-1,4-beta-mannosidase B] Caldalkalibacillus mannanilyticus (strain DSM 16130/CIP 109019/JCM 10596/AM-001) (Bacillus mannanilyticus)

MKVYKKVAFVMAFIMFFSVLPTISMSSEANGAALSNPNANQTTKNVYSWLANLPNK
SNKRVVSGHFGGYSDSTLAWIKQCARELTGKMPGILSCDYKNWQTRLYVADQISYG
CNQELINFWNQGGLVTISVHMPNPGFHSGENYKTILPTSQFQNLTNHRTTEGRRWKD
MLDKMADGLDELQNNGVTVLFRPLHEMNGEWFWWGAEGYNQFDQTRANAYISA
WRDMYQYFTHERKLNNLIWVYSPDVYRDHVTSYYPGANYVDIVALDSYHPDPHSL
TDQYNRMIALDKPFAFAEIGPPESMAGSFDYSNYIQAIKQKYPRTVYFLAWNDKWSP
HNNRGAWDLFNDSWVVNRGEIDYGQSNPATVLYDFENNTLSWSGCEFTDGGPWTS
NEWSANGTQSLKADVVLGNNSYHLQKTVNRNLSSFKNLEIKVSHSSWGNVGSGMT
ARVFVKTGSAWRWNAGEFCQFAGKRTTALSIDLTKVSNLHDVREIGVEYKAPANSN
GKTAIYLDHVTVR

Beta-mannanase/endoglucanase A [Includes: Mannan endo-1,4-beta-mannosidase A (EC 3.2.1.78) (Beta-mannanase) (Endo-1,4-mannanase); Endo-1,4-beta-glucanase (EC 3.2.1.4) (Cellulase)] Caldicellulosiruptor saccharolyticus (Caldocellum saccharolyticum)

MRLKTKIRKKWLSVLCTVVFLLNILFIANVTILPKVGAATSNDGVVKIDTSTLIGTNH
AHCWYRDRLDTALRGIRSWGMNSVRVVLSNGYRWTKIPASEVANIISLSRSLGFKAII
LEVHDTTGYGEDGAACSLAQAVEYWKEIKSVLDGNEDFVIINIGNEPYGNNNYQNW
VNDTKNAIKALRDAGFKHTIMVDAPNWGQDWSNTMRDNAQSIMEADPLRNLVFSI
HMYGVYNTASKVEEYIKSFVDKGLPLVIGEFGHQHTDGDPDEEAIVRYAKQYKIGLF
SWSWCGNSSYVGYLDMVNNWDPNNPTPWGQWYKTNAIGTSSTPTPTSTVTPTPTPT
PTPTPTVTATPTPTPTPVSTPATSGQIKVLYANKETNSTTNTIRPWLKVVNSGSSSIDLS
RVTIRYWYTVDGERAQSAISDWAQIGASNVTFKFVKLSSSVSGADYYLEIGFKSGAG
QLQPGKDTGEIQMRFNKDDWSNYNQGNDWSWIQSMTSYGENEKVTAYIDGVLVW
GQEPSGATPAPAPTATPTPTPTVTPTPTVTPTPTVTATPTPTPTPTPTPVSTPATGGQIK
VLYANKETNSTTNTIRPWLKVVNSGSSSIDLSRVTIRYWYTVDGERAQSAISDWAQI
GASNVTFKFVKLSSSVSGADYYLEIGFKSGAGQLQPGKDTGEIQIRFNKSDWSNYNQ
GNDWSWIQSMTSYGENEKVTAYIDGVLVWGQEPSGTTPSPTSTPTVTVTPTPTPTPTP
TPTPTVTPTPTVTPTPTVTATPTPTPTPIPTVTPLPTISPSPSVVEITINTNAGRTQISPYIY
GANQDIEGVVHSARRLGGNRLTGYNWENNFSNAGNDWYHSSDDYLCWSMGISGE
DAKVPAAVVSKFHEYSLKNNAYSAVTLQMAGYVSKDNYGTVSENETAPSNRWAEV
KFKKDAPLSLNPDLNDNFVYMDEFINYLINKYGMASSPTGIKGYILDNEPDLWASTH
PRIHPNKVTCKELIEKSVELAKVIKTLDPSAEVEGYASYGFMGYYSLQDAPDWNQVK
GEHRWFISWYLEQMKKASDSFGKRLLDVLDLHWYPEARGGNIRVCFDGENDTSKE
VVIARMQAPRTLWDPTYKTSVKGQITAGENSWINQWFSDYLPIIPNVKADIEKYYPG
TKLAISEFDYGGRNHISGGIALADVLGIFGKYGVNFAARWGDSGSYAAAAYNIYLNY
DGKGSKYGNTNVSANTSDVENMPVYASINGQDDSELHIILINRNYDQKLQVKINITST
PKYTKAEIYGFDSNSPEYKKMGNIDNIESNVFTLEVPKFNGVSHSITLDFNVSIKIIQNE
VIKFIRNLVFMRALV

Mannan endo-1,4-beta-mannosidase (EC 3.2.1.78) (Mannanase 26A) (Man26A) (Mannanase A) (ManA) Cellvibrio japonicus (strain Ueda107) (Pseudomonas fluorescens subsp. cellulosa)

MKTITTARLPWAAQSFALGICLIALLGCNHAANKSSASRADVKPVTVKLVDSQATM
ETRSLFAFMQEQRRHSIMFGHQHETTQGLTITRTDGTQSDTFNAVGDFAAVYGWDT
LSIVAPKAEGDIVAQVKKAYARGGIITVSSHFDNPKTDTQKGVWPVGTSWDQTPAV
VDSLPGGAYNPVLNGYLDQVAEWANNLKDEQGRLIPVIFRLYHENTGSWFWWGDK
QSTPEQYKQLFRYSVEYLRDVKGVRNFLYAYSPNNFWDVTEANYLERYPGDEWVD
VLGFDTYGPVADNADWFRNVVANAALVARMAEARGKIPVISEIGIRAPDIEAGLYD
NQWYRKLISGLKADPDAREIAFLLVWRNAPQGVPGPNGTQVPHYWVPANRPENINN
GTLEDFQAFYADEFTAFNRDIEQVYQRPTLIVK

Mannan endo-1,4-beta-mannosidase (EC 3.2.1.78) Clostridium acetobutylicum (strain ATCC 824/DSM 792/JCM 1419/LMG 5710/VKM B-1787)

MTLKNPKKLFLSIFAVLYIISAANPTKVSAFFSTCKAGVFLGWDRNIDTSIAYTDGYS
NRLDIISYDLCNWNGNNDSSSISWDSASTIRDLQKPNIPLIKQYKGPYAHLIKNVIISA
HMPCPTGGTSNGSISPQDFATLANTKIQDINSSSPWFLQNYKAIVSKVGDGLEYYGH
VGTVYFRPFHEVNGNWFWWGDKDVSQYKQLWINLHDYLVSKNSGCRGLTWLKFC
YAPNSGKNAALYYPGDDYVDMVGMDAYSDNPSTDTYVKRAYAELTGGTYNGVTY
PGIDKPFGFPELGPNVGGDYVDNKNRLIKKFDYKRWSDAIKLNYPKASYFIIWNGGY
EPRNNDNGTSLFNMSY

Mannan endo-1,4-beta-mannosidase (EC 3.2.1.78) (Endo-(1,4)-beta-mannanase) Rhodothermus marinus (strain ATCC 43812/DSM 4252/R-10) (Rhodothermus obamensis)

MTLLLVWLIFTGVAGEIRLEAEDGELLGVAVDSTLTGYSGRGYVTGFDAPEDSVRFS
FEAPRGVYRVVFGVSFSSRFASYALRVDDWHQTGSLIKRGGGFFEASIGEIWLDEGA
HTMAFQLMNGALDYVRLEPVSYGPPARPPAQLSDSQATASAQALFAFLLSEYGRHIL
AGQQQNPYRRDFDAINYVRNVTGKEPALVSFDLIDYSPTREAHGVVHYQTPEDWIA
WAGRDGIVSLMWHWNAPTDLIEDPSQDCYWWYGFYTRCTTFDVAAALADTSSERY
RLLLRDIDVIAAQLQKFQQADIPVLWRPLHEAAGGWFWWGAKGPEPFKQLWRLLY
ERLVHHHGLHNLIWVYTHEPGAAEWYPGDAYVDIVGRDVYADDPDALMRSDWNE
LQTLFGGRKLVALTETGTLPDVEVITDYGIWWSWFSIWTDPFLRDVDPDRLTRVYHS
ERVLTRDELPDWRSYVLHATTVQPAGDLALAVYPNPGAGRLHVEVGLPVAAPVVV
EVFNLLGQRVFQYQAGMQPAGLWRRAFELALAPGVYLVQVRAGNLVARRRWVSV
R

Mannan endo-1,4-beta-mannosidase (EC 3.2.1.78) (1,4-beta-D-mannan mannanohydrolase) (Beta-mannanase) Streptomyces lividans

MRNARSTLITTAGMAFAVLGLLFALAGPSAGRAEAAAGGIHVSNGRVVEGNGSAFV
MRGVNHAYTWYPDRTGSIADIAAKGANTVRVVLSSGGRWTKTSASEVSALIGQCKA
NKVICVLEVHDTTGYGKDGATSLDQAGDYWVGVKSAAWRAQEDYVVVNIGNEPF
GNTNYAAWTDATKSAIGKLRGAGLGHALMVDAPNWGQDWSGTMRSNAASVFASD
PDRNTVFSIHMYGVYDTAAEVRDYLNAFVGNGLPIVVGEFGDQHSDGNPDEDAIMA
TAQSLGVGYLGWSWSGNGGGVEYLDMVNGFDPNSLTSWGNRILYGSNGIAATSRT
ATVYGGGGGSTGGTAPNGYPYCVNGGASDPDGDGWGWENSRSCVVRGSAADH

Mannan endo-1,4-beta-mannosidase man26A (EC 3.2.1.78) (Endo-(1,4)-beta-mannanase man26A) Aspergillus niger (strain ATCC MYA-4892/CBS 513.88/FGSC A1513)

MFAKLSLLSLLESSAALGASNQTLSYGNIDKSATPEARALLKYIQLQYGSHYISGQQD
IDSWNWVEKNIGVAPAILGSDFTYYSPSAVAHGGKSHAVEDVIQHAGRNGINALVW
HWYAPTCLLDTAKEPWYKGFYTEATCFNVSEAVNDHGNGTNYKLLLRDIDAIAAQI
KRLDQAKVPILFRPLHEPEGGWFWWGAQGPAPFKKLWDILYDRITRYHNLHNMVW
VCNTADPAWYPGNDKCDIATIDHYPAVGDHGVAADQYKKLQTVINNERVLAMAE
VGPIPDPDKQARENVNWAYWMVWSGDFIEDGKQNPNQFLHKVYNDTRVVALNWE
GA

Mannan endo-1,4-beta-mannosidase C (EC 3.2.1.78) (Endo-beta-1,4-mannanase C) Emericella nidulans (strain FGSC Δ4/ATCC 38163/CBS 112.46/NRRL 194/M139) (Aspergillus nidulans)

MIFSTLLSLALLATTATARKGFVTTKGDKFQLDGKDFYFAGSNAYYFPFNNNQTDVE
LGLSAAKKAGLLVFRTWGFNDKNVTYIEDGLPQYGGEGAGTTEVVFQWWQNGTST
IDLEPFDKVVNAAAKTGIKLIVTLVNNWADYGGMDVYTVNLGGQYHDDFYRLPQIK
KAYKRYVKEMVTRYRNSPAIMAWELANEPRCGADGVRNLPASDECTPELLTSWIDE
MSTYVKRLDPHHLVTWGGEGGFNYDSDDWAYNGSDGGDFEAELKLKNIDFGVFHS
YPDWWSKTVEWTNKWIVDHARAARRVGKPVVHEEYGWLTPQGRLDNLGTVSNIT
RLEAVGGWQSISLREKMSDMFWQFGYSGYSYGRNHDDGFTIYLDDAEAQELVYKH
AKEVNKLNRRR

Probable mannan endo-1,4-beta-mannosidase A (EC 3.2.1.78) (Endo-beta-1,4-mannanase A) Aspergillus oryzae (strain ATCC 42149/RIB 40) (Yellow koji mold)

MKLNPSLLTAAGLVSAQLASALPQASSSTVSPSPSPSATPGSFVTTSGLNFVIDGKTG
YFAGSNSYWIGFQKNNDDVDLVFSHLQESGLKILRVWGFNDVNQKPTDGSVYYHLL
ADGTATVNEGEDGLQRLDYVVSSAEKHGIKLIINFVNFWDDYGGINAYVKAFGGSK
EGFYTNDAMQAAYRAYIKAVISRYSDSTAIFAWELANEPRCQGCETTVLYNWIESTS
QYIKSLDSKHLVCIGDEGFGLDTGSDGSYPYQYSEGSDFAKNLAIPTIDFGTFHLYPSS
WGTTNDWGNGWVTSHGAACKAAGKPCLFEEYGVTSDHCAVEKPWQNTALNTTAI
SGDLYWQYGDQLSGGPSPDDGNTFYYGTDDFKCLVTDHIAAINSRK

Mannan endo-1,4-beta-mannosidase A (EC 3.2.1.78) (Beta-mannanase 5A) (Man5A) (Beta-mannanase I/II) (BMANI) (BMANII) (Endo-beta-1,4-mannanase A)Hypocrea jecorina (strain ATCC 56765/BCRC 32924/NRRL 11460/Rut C-30) (Trichoderma reesei)

MMMLSKSLLSAATAASALAAVLQPVPRASSFVTISGTQFNIDGKVGYFAGTNCYWC
SFLTNHADVDSTFSHISSSGLKVVRVWGFNDVNTQPSPGQIWFQKLSATGSTINTGA
DGLQTLDYVVQSAEQHNLKLIIPFVNNWSDYGGINAYVNAFGGNATTWYTNTAAQ
TQYRKYVQAVVSRYANSTAIFAWELGNEPRCNGCSTDVIVQWATSVSQYVKSLDSN
HLVTLGDEGLGLSTGDGAYPYTYGEGTDFAKNVQIKSLDFGTFHLYPDSWGTNYTW
GNGWIQTHAAACLAAGKPCVFEEYGAQQNPCTNEAPWQTTSLTTRGMGGDMFWQ
WGDTFANGAQSNSDPYTVWYNSSNWQCLVKNHVDAINGGTTTPPPVSSTTTTSSRT
SSTPPPPGGSCSPLYGQCGGSGYTGPTCCAQGTCIYSNYWYSQCLNT

Suitable exogenous or heterologous nucleic acid sequences encoding a protein with serine protease activity may also include nucleic acid sequences which can be derived as a result of the degenerate genetic code from the amino acid sequences identified above as well as functional equivalents with serine protease activity.

Suitable exogenous or heterologous nucleic acid sequences that that encode a protein with cellulase activity include, but are not limited to, a nucleic acid sequences that code for proteins or polypeptides having cellulase activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Endoglucanase D (EGD) (EC 3.2.1.4) (Cellulase D) (Endo-1,4-beta-glucanase) Acetivihrin thermocellus (Imugateiclostridium thermocellum) (Clostridium thermocellum)

SLTGVFPSGLIETKVSAAKITENYQFDSRIRLNSIGFIPNHSKKATIAANCSTFYVVKED
GTIVYTGTATSMFDNDTKETVYIADFSSVNEEGTYYLAVPGVGKSVNFKIAMNVYE
DAFKTAMLGMYLLRCGTSVSATYNGIHYSHGPCHTNDAYLDYINGQHTKKDSTKG
WHDAGDYNKYVVNAGITVGSMFLAWEHFKDQLEPVALEIPEKNNSIPDFLDELKYEI
DWILTMQYPDGSGRVAHKVSTRNFGGFIMPENEHDERFFVPWSSAATADFVAMTA
MAARIFRPYDPQYAEKCINAAKVSYEFLKNNPANVFANQSGFSTGEYATVSDADDR
LWAAAEMWETLGDEEYLRDFENRAAQFSKKIEADFDWDNVANLGMFTYLLSERPG
KNPALVQSIKDSLLSTADSIVRTSQNHGYGRTLGTTYYWGCNGTVVRQTMILQVAN
KISPNNDYVNAALDAISHVFGRNYYNRSYVTGLGINPPMNPHDRRSGADGIWEPWP
GYLVGGGWPGPKDWVDIQDSYQTNEIAINWNAALIYALAGFVNYNSPQNEVLYGD
VNDDGKVNSTDLTLLKRYVLKAVSTLPSSKAEKNADVNRDGRVNSSDVTILSRYLIR
VIEKLPI

Endoglucanase C (EC 3.2.1.4) (Cellulase C) (Endo-1,4-beta-glucanase C) (EgC) Acetivibrio thermocellus (Hungateiclostridium thermocellum) (Clostridium thermocellum)

MVSFKAGINLGGWISQYQVFSKEHFDTFITEKDIETIAEAGFDHVRLPFDYPIIESDDN
VGEYKEDGLSYIDRCLEWCKKYNLGLVLDMHHAPGYRFQDFKTSTLFEDPNQQKRF
VDIWRFLAKRYINEREHIAFELLNEVVEPDSTRWNKLMLEYIKAIREIDSTMWLYIGG
NNYNSPDELKNLADIDDDYIVYNFHFYNPFFFTHQKAHWSESAMAYNRTVKYPGQY
EGIEEFVKNNPKYSFMMELNNLKLNKELLRKDLKPAIEFREKKKCKLYCGEFGVIAI
ADLESRIKWHEDYISLLEEYDIGGAVWNYKKMDFEIYNEDRKPVSQELVNILARRKT

Endoglucanase (EC 3.2.1.4) Acetivibrio thermocellus (Hungateiclostridium thermocellum) (Clostridium thermocellum)

YILPQPDVRVNQVGYLPEGKKVATVVCNSTQPVKWQLKNAAGVVVLEGYTEPKGL
DKDSQDYVHWLDFSDFATEGIGYYFELPTVNSPTNYSHPFDIRKDIYTQMKYDALAF
FYHKRSGIPIEMPYAGGEQWTRPAGHIGIEPNKGDTNVPTWPQDDEYAGIPQKNYTK
DVTGGWYDAGDHGKYVVNGGIAVWTLMNMYERAKIRGLDNWGPYRDGGMNIPE
QNNGYPDILDEARWEIEFFKKMQVTEKEDPSIAGMVHHKIHDFRWTALGMLPHEDP
QPRYLRPVSTAATLNFAATLAQSARLWKDYDPTFAADCLEKAEIAWQAALKHPDIY
AEYTPGSGGPGGGPYNDDYVGDEFYWAACELYVTTGKDEYKNYLMNSPHYLEMP
AKMGENGGANGEDNGLWGCFTWGTTQGLGTITLALVENGLPATDIQKARNNIAKA
ADRWLENIEEQGYRLPIKQAEDERGGYPWGSNSFILNQMIVMGYAYDFTGDSKYLD
GMFDGISYLLGRNAMDQSYVTGYGERPLQNPHDRFWTPQTSKRFPAPPPGIISGGPN
SRFEDPTINAAVKKDTPPQKCFIDHTDSWGTNEITVNWNAPFAWVTAYLDE

Cellulase/esterase CelE (CtCel5C-CE2) [Includes: Cellulase E (EC 3.2.1.4) (CtCel5C) (Endo-1,4-beta-glucanase E) (EGE) (Endoglucanase E); Acetylxylan esterase/glucomannan deacetylase (EC 3.1.1.→(EC 3.1.1.72) (CtCE2)]Acetivibrio thermocellus (strain ATCC 27405/DSM 1237/JCM 9322/NBRC 103400/NCIMB 10682/NRRL B-4536/VPI 7372) (Clostridium thermocellum)

MKKIVSLVCVLVMLVSILGSFSVVAASPVKGFQVSGTKLLDASGNELVMRGMRDIS
AIDLVKEIKIGWNLGNTLDAPTETAWGNPRTTKAMIEKVREMGFNAVRVPVTWDTH
IGPAPDYKIDEAWLNRVEEVVNYVLDCGMYAIINVHHDNTWIIPTYANEQRSKEKLV
KVWEQIATRFKDYDDHLLFETMNEPREVGSPMEWMGGTYENRDVINRFNLAVVNTI
RASGGNNDKRFILVPTNAATGLDVALNDLVIPNNDSRVIVSIHAYSPYFFAMDVNGT
SYWGSDYDKASLTSELDAIYNRFVKNGRAVIIGEFGTIDKNNLSSRVAHAEHYAREA
VSRGIAVFWWDNGYYNPGDAETYALLNRKTLSWYYPEIVQALMRGAGVEPLVSPT
PTPTLMPTPSPTVTANILYGDVNGDGKINSTDCTMLKRYILRGIEEFPSPSGIIAADVN
ADLKINSTDLVLMKKYLLRSIDKFPAEDSQTPDEDNPGILYNGRFDFSDPNGPKCAW
SGSNVELNFYGTEASVTIKSGGENWFQAIVDGNPLPPFSVNATTSTVKLVSGLAEGA
HHLVLWKRTEASLGEVQFLGFDFGSGKLLAAPKPLERKIEFIGDSITCAYGNEGTSKE
QSFTPKNENSYMSYAAITARNLNASANMIAWSGIGLTMNYGGAPGPLIMDRYPYTLP
YSGVRWDFSKYVPQVVVINLGTNDFSTSFADKTKFVTAYKNLISEVRRNYPDAHIFC
CVGPMLWGTGLDLCRSYVTEVVNDCNRSGDLKVYFVEFPQQDGSTGYGEDWHPSI
ATHQLMAERLTAEIKNKLGW

Endoglucanase 1 (EC 3.2.1.4) (Cellulase I) (Endo-1,4-beta-glucanase) (Endoglucanase I) (EGI) Acetivibrio thermocellus (strain ATCC 27405/DSM 1237/JCM 9322/NBRC 103400/NCIMB 10682/NRRL B-4536/VPI 7372) (Clostridium thermocellum)

MRLVNSLGRRKILLILAVIVAFSTVLLFAKLWGRKTSSTLDEVGSKTHGDLTAENKN
GGYLPEEEIPDQPPATGAFNYGEALQKAIFFYECQRSGKLDPSTLRLNWRGDSGLDD
GKDAGIDLTGGWYDAGDHVKFNLPMSYSAAMLGWAVYEYEDAFKQSGQYNHILN
NIKWACDYFIKCHPEKDVYYYQVGDGHADHAWWGPAEVMPMERPSYKVDRSSPG
STVVAETSAALAIASIIFKKVDGEYSKECLKHAKELFEFADTTKSDDGYTAANGFYNS
WSGFYDELSWAAVWLYLATNDSSYLDKAESYSDKWGYEPQTNIPKYKWAQCWDD
VTYGTYLLLARIKNDNGKYKEAIERHLDWWTTGYNGERITYTPKGLAWLDQWGSL
RYATTTAFLACVYSDWENGDKEKAKTYLEFARSQADYALGSTGRSFVVGFGENPPK
RPHHRTAHGSWADSQMEPPEHRHVLYGALVGGPDSTDNYTDDISNYTCNEVACDY
NAGFVGLLAKMYKLYGGSPDPKFNGIEEVPEDEIFVEAGVNASGNNFIEIKAIVNNKS
GWPARVCENLSFRYFINIEEIVNAGKSASDLQVSSSYNQGAKLSDVKHYKDNIYYVE
VDLSGTKIYPGGQSAYKKEVQFRISAPEGTVFNPENDYSYQGLSAGTVVKSEYIPVY
DAGVLVFGREPGSASKSTSKDNGLSKATPTVKTESQPTAKHTQNPASDFKTPANQNS
VKKDQGIKGEVVLQYANGNAGATSNSINPRFKIINNGTKAINLSDVKIRYYYTKEGG
ASQNFWCDWSSAGNSNVTGNFFNLSSPKEGADTCLEVGFGSGAGTLDPGGSVEVQI
RFSKEDWSNYNQSNDYSFNPSASDYTDWNRVTLYISNKLVYGKEP

Endoglucanase A (EGA) (EC 3.2.1.4) (Cellulase A) (Endo-1,4-beta-glucanase) Acetivibrio thermocellus (strain ATCC 27405/DSM 1237/JCM 9322/NBRC 103400/NCIMB 10682/NRRL B-4536/VPI 7372) (Clostridium thermocellum)

MKNVKKRVGVVLLILAVLGVYMLAMPANTVSAAGVPFNTKYPYGPTSIADNQSEV
TAMLKAEWEDWKSKRITSNGAGGYKRVQRDASTNYDTVSEGMGYGLLLAVCFNE
QALFDDLYRYVKSHFNGNGLMHWHIDANNNVTSHDGGDGAATDADEDIALALIFA
DKLWGSSGAINYGQEARTLINNLYNHCVEHGSYVLKPGDRWGGSSVTNPSYFAPA
WYKVYAQYTGDTRWNQVADKCYQIVEEVKKYNNGTGLVPDWCTASGTPASGQSY
DYKYDATRYGWRTAVDYSWFGDQRAKANCDMLTKFFARDGAKGIVDGYTIQGSKI
SNNHNASFIGPVAAASMTGYDLNFAKELYRETVAVKDSEYYGYYGNSLRLLTLLYIT
GNFPNPLSDLSGQPTPPSNPTPSLPPQVVYGDVNGDGNVNSTDLTMLKRYLLKSVTN
INREAADVNRDGAINSSDMTILKRYLIKSIPHLPY

Endoglucanase D (EGD) (EC 3.2.1.4) (Cellulase D) (Endo-1,4-beta-glucanase) Acetivibrio thermocellus (strain ATCC 27405/DSM 1237/JCM 9322/NBRC 103400/NCIMB 10682/NRRL B-4536/VPI 7372) (Clostridium thermocellum)

MSRMTLKSSMKKRVLSLLIAVVFLSLTGVFPSGLIETKVSAAKITENYQFDSRIRLNSI
GFIPNHSKKATIAANCSTFYVVKEDGTIVYTGTATSMFDNDTKETVYIADFSSVNEEG
TYYLAVPGVGKSVNFKIAMNVYEDAFKTAMLGMYLLRCGTSVSATYNGIHYSHGP
CHTNDAYLDYINGQHTKKDSTKGWHDAGDYNKYVVNAGITVGSMFLAWEHFKDQ
LEPVALEIPEKNNSIPDFLDELKYEIDWILTMQYPDGSGRVAHKVSTRNFGGFIMPEN
EHDERFFVPWSSAATADFVAMTAMAARIFRPYDPQYAEKCINAAKVSYEFLKNNPA
NVFANQSGFSTGEYATVSDADDRLWAAAEMWETLGDEEYLRDFENRAAQFSKKIE
ADFDWDNVANLGMFTYLLSERPGKNPALVQSIKDSLLSTADSIVRTSQNHGYGRTL
GTTYYWGCNGTVVRQTMILQVANKISPNNDYVNAALDAISHVFGRNYYNRSYVTG
LGINPPMNPHDRRSGADGIWEPWPGYLVGGGWPGPKDWVDIQDSYQTNEIAINWN
AALIYALAGFVNYNSAQNEVLYGDVNDDGKVNSTDLTLLKRYVLKAVSTLPSSKAE
KNADVNRDGRVNSSDVTILSRYLIRVIEKLPI

Endoglucanase H (EC 3.2.1.4) (Cellulase H) (Endo-1,4-beta-glucanase H) (EgH) Acetivibrio thermocellus (strain ATCC 27405/DSM 1237/JCM 9322/NBRC 103400/NCIMB 10682/NRRL B-4536/VPI 7372) (Clostridium thermocellum)

MKKRLLVSFLVLSIIVGLLSFQSLGNYNSGLKIGAWVGTQPSESAIKSFQELQGRKLDI
VHQFINWSTDFSWVRPYADAVYNNGSILMITWEPWEYNTVDIKNGKADAYITRMA
QDMKAYGKEIWLRPLHEANGDWYPWAIGYSSRVNTNETYIAAFRHIVDIFRANGAT
NVKWVFNVNCDNVGNGTSYLGHYPGDNYVDYTSIDGYNWGTTQSWGSQWQSFDQ
VFSRAYQALASINKPIIIAEFASAEIGGNKARWITEAYNSIRTSYNKVIAAVWFHENKE
TDWRINSSPEALAAYREAIGAGSSNPTPTPTWTSTPPSSSPKAVDPFEMVRKMGMGT
NLGNTLEAPYEGSWSKSAMEYYFDDFKAAGYKNVRIPVRWDNHTMRTYPYTIDKA
FLDRVEQVVDWSLSRGFVTIINSHHDDWIKEDYNGNIERFEKIWEQIAERFKNKSENL
LFEIMNEPFGNITDEQIDDMNSRILKIIRKTNPTRIVIIGGGYWNSYNTLVNIKIPDDPY
LIGTFHYYDPYEFTHKWRGTWGTQEDMDTVVRVFDFVKSWSDRNNIPVYFGEFAV
MAYADRTSRVKWYDFISDAALERGFACSVWDNGVFGSLDNDMAIYNRDTRTFDTEI
LNALFNPGTYPSYSPKPSPTPRPTKPPVTPAVGEKMLDDFEGVLNWGSYSGEGAKVS
TKIVSGKTGNGMEVSYTGTTDGYWGTVYSLPDGDWSKWLKISFDIKSVDGSANEIR
FMIAEKSINGVGDGEHWVYSITPDSSWKTIEIPFSSFRRRLDYQPPGQDMSGTLDLDNI
DSIHFMYANNKSGKFVVDNIKLIGATSDPTPSIKHGDLNFDNAVNSTDLLMLKRYILK
SLELGTSEQEEKFKKAADLNRDNKVDSTDLTILKRYLLKAISEIPI

Cellulase/esterase CelE (CtCel5C-CE2) [Includes: Cellulase E (EC 3.2.1.4) (CtCel5C) (Endo-1,4-beta-glucanase E) (EGE) (Endoglucanase E); Acetylxylan esterase/glucomannan deacetylase (EC 3.1.1.→(EC 3.1.1.72) (CtCE2)]Acetivibrio thermocellus (strain ATCC 27405/DSM 1237/JCM 9322/NBRC 103400/NCIMB 10682/NRRL B-4536/VPI 7372) (Clostridium thermocellum)

MKKIVSLVCVLVMLVSILGSFSVVAASPVKGFQVSGTKLLDASGNELVMRGMRDIS
AIDLVKEIKIGWNLGNTLDAPTETAWGNPRTTKAMIEKVREMGFNAVRVPVTWDTH
IGPAPDYKIDEAWLNRVEEVVNYVLDCGMYAIINVHHDNTWIIPTYANEQRSKEKLV
KVWEQIATRFKDYDDHLLFETMNEPREVGSPMEWMGGTYENRDVINRFNLAVVNTI
RASGGNNDKRFILVPTNAATGLDVALNDLVIPNNDSRVIVSIHAYSPYFFAMDVNGT
SYWGSDYDKASLTSELDAIYNRFVKNGRAVIIGEFGTIDKNNLSSRVAHAEHYAREA
VSRGIAVFWWDNGYYNPGDAETYALLNRKTLSWYYPEIVQALMRGAGVEPLVSPT
PTPTLMPTPSPTVTANILYGDVNGDGKINSTDCTMLKRYILRGIEEFPSPSGIIAADVN
ADLKINSTDLVLMKKYLLRSIDKFPAEDSQTPDEDNPGILYNGRFDFSDPNGPKCAW
SGSNVELNFYGTEASVTIKSGGENWFQAIVDGNPLPPFSVNATTSTVKLVSGLAEGA
HHLVLWKRTEASLGEVQFLGFDFGSGKLLAAPKPLERKIEFIGDSITCAYGNEGTSKE
QSFTPKNENSYMSYAAITARNLNASANMIAWSGIGLTMNYGGAPGPLIMDRYPYTLP
YSGVRWDFSKYVPQVVVINLGTNDFSTSFADKTKFVTAYKNLISEVRRNYPDAHIFC
CVGPMLWGTGLDLCRSYVTEVVNDCNRSGDLKVYFVEFPQQDGSTGYGEDWHPSI
ATHQLMAERLTAEIKNKLGW

Endoglucanase E1 (EC 3.2.1.4) (Cellulase E1) (Endo-1,4-beta-glucanase E1) (Endocellulase E1)Acidothermus cellulolyticus (strain ATCC 43068/DSM 8971/11B)

MPRALRRVPGSRVMLRVGVVVAVLALVAALANLAVPRPARAAGGG
YWHTSGREILDANNVPVRIAGINWFGFETCNYVVHGLWSRDYRSM
LDQIKSLGYNTIRLPYSDDILKPGTMPNSINFYQMNQDLQGLTSL
QVMDKIVAYAGQIGLRIILDRHRPDCSGQSALWYTSSVSEATWIS
DLQALAQRYKGNPTVVGFDLHNEPHDPACWGCGDPSIDWRLAAER
AGNAVLSVNPNLLIFVEGVQSYNGDSYWWGGNLQGAGQYPVVLNV
PNRLVYSAHDYATSVYPQTWFSDPTFPNNMPGIWNKNWGYLFNQN
IAPVWLGEFGTTLQSTTDQTWLKTLVQYLRPTAQYGADSFQWTFW
SWNPDSGDTGGILKDDWQTVDTVKDGYLAPIKSSIFDPVGASASP
SSQPSPSVSPSPSPSPSASRTPTPTPTPTASPTPTLTPTATPTPT
ASPTPSPTAASGARCTASYQVNSDWGNGFTVTVAVTNSGSVATKT
WTVSWTFGGNQTITNSWNAAVTQNGQSVTARNMSYNNVIQPGQNT
TFGFQASYTGSNAAPTVACAAS

Endoglucanase E1 (EC 3.2.1.4) (Cellulase E1) (Endo-1,4-beta-glucanase E1) (Endocellulase E1)Acidothermus cellulolyticus (strain ATCC 43068/DSM 8971/11B)

MPRALRRVPGSRVMLRVGVVVAVLALVAALANLAVPRPARAAGGG
YWHTSGREILDANNVPVRIAGINWFGFETCNYVVHGLWSRDYRSM
LDQIKSLGYNTIRLPYSDDILKPGTMPNSINFYQMNQDLQGLTSL
QVMDKIVAYAGQIGLRIILDRHRPDCSGQSALWYTSSVSEATWIS
DLQALAQRYKGNPTVVGFDLHNEPHDPACWGCGDPSIDWRLAAER
AGNAVLSVNPNLLIFVEGVQSYNGDSYWWGGNLQGAGQYPVVLNV
PNRLVYSAHDYATSVYPQTWFSDPTFPNNMPGIWNKNWGYLFNQN
IAPVWLGEFGTTLQSTTDQTWLKTLVQYLRPTAQYGADSFQWTFW
SWNPDSGDTGGILKDDWQTVDTVKDGYLAPIKSSIFDPVGASASP
SSQPSPSVSPSPSPSPSASRTPTPTPTPTASPTPTLTPTATPTPT
ASPTPSPTAASGARCTASYQVNSDWGNGFTVTVAVTNSGSVATKT
WTVSWTFGGNQTITNSWNAAVTQNGQSVTARNMSYNNVIQPGQNT
TFGFQASYTGSNAAPTVACAAS

Endoglucanase (EC 3.2.1.4) Alicyclobacillus acidocaldarius subsp. acidocaldarius (Bacillus acidocaldarius)

MPSRVPKSIFYNQVGYLISGDKRFWIQAHEPQPFALRTPEGQAVF
AGMTKPVGGNWYVGDFTALRVPGTYTLTVGTLEARVVIHRRAYRD
VLEAMLRFFDYQLCGVVLPEDEAGPWAHGACHTSDAKVFGTERAL
ACPGGWHDAGDYGKYTVPAAKAVADLLLAHEYFPAALAHVRPMRS
VHRAPHLPPALEVAREEIAWLLTMQDPATGGVYHKVTTPSFPPLD
TRPEDDDAPLVLSPISYAATATFCAAMAHAALVYRPFDPALSSCC
ADAARRAYAWLGAHEMQPFHNPDGILTGEYGDAELRDELLWASCA
LLRMTGDSAWARVCEPLLDLDLPWELGWADVALYGVMDYLRTPRA
AVSDDVRNKVKSRLLRELDALAAMAESHPFGIPMRDDDFIWGSNM
VLLNRAMAFLLAEGVGVLHPAAHTVAQRAADYLFGANPLGQCYVT
GFGQRPVRHPHHRPSVADDVDHPVPGMVVGGPNRHLQDEIARAQL
AGRPAMEAYIDHQDSYSTNEVAVYWNSPAVFVIAALLEARGR

cellulase (EC 3.2.1.4) Aliivibrio fischeri (strain ATCC 700601/ES 114) (Vibrio fischeri)

MKMQLTVLSLLIGTLTCSSSAIAQDENYTTPIVSSAALTKENQCE
WGQWESFKQHYIENGRVVDNSDPRLITTSEGQSYALFFALIANDK
KTFDELLGWTELHLAGGDLTAQLPAWLWGRQPDGSQGILDSNSAA
DSDLWIAYSLLEAGRLWDNHYYQSLGHLLASRILRDETIKVSGLG
TVLLPGKVGFVLGKNHVRLNPSYVPLQLLTRMNTVFPSYQWEEIY
QSSAKLLKETMPKGYSPDWVEWDKTQFKKDSKTQSVGSYNAIRVY
LWAGMLPDSDPNKALLLDKMKPLVRVIERNKGMPETINVLTGKGK
NQGGVGMNAAILPLLSSLDSNTNAVEYEKKIQAELSKIESDYYYN
SVLTLFGLGWYQDLYSFNDDGSVTPKWVNVCQ

Probable endo-beta-1,4-glucanase D (Endoglucanase D) (EC 3.2.1.4) (Carboxymethylcellulase D) (Cellulase D) Aspergillus fumigatus (strain CBS 144.89/FGSC A 1163/CEΔ10) (Neosartorya fumigata)

MKSTFGLLALAAAAKLVSAHATVHAVWINDVDQGAGNSADGYIRS
PPNNSPITDVTSTDMTCNVNGKNPVAKTLSVKAGDKVTFEWHHDT
RSDSDDIIASSHMGPVMVYMAPTEKGTAGNGWVKIAEEGYSNGKW
AVANLIANRGKHSITVPDVPAGEYLLRPEIIALHEGNRQGGAQFY
MECVQVKVTSAGTKTLPAGVSIPGAYKATDPGVLFDMYNSFTSYP
IPGPAVWDGSSSGSSGSSGSSPATTTAPAVSVTAVPTKEAPVDTS
ATPTTFVTATKPATTAAPAAPSASSGSNSGSDSCNSGSASGSVKI
YGQCGGQNYSGPTSCEAGLICKEWNPYYHQCVSA

Probable endo-beta-1,4-glucanase B (Endoglucanase B) (EC 3.2.1.4) (Carboxymethylcellulase B) (Cellulase B) Aspergillus niger (strain ATCC MYA-4892/CBS 513.88/FGSC A1513)

MKFQSTLLLAAAAGSALAVPHGSGHKKRASVFEWFGSNESGAEFG
TNIPGVWGTDYIFPDPSTISTLIGKGMNFFRVQFMMERLLPDSMT
GSYDEEYLANLTTVVKAVTDGGAHALIDPHNYGRYNGEIISSTSD
FQTFWQNLAGQYKDNDLVMFDTNNEYYDMDQDLVLNLNQAAINGI
RAAGASQYIFVEGNSWTGAWTWVDVNDNMKNLTDPEDKIVYEMHQ
YLDSDGSGTSETCVSGTIGKERITDATQWLKDNKKVGFIGEYAGG
SNDVCRSAVSGMLEYMANNTDVWKGASWWAAGPWWGDYIFSLEPP
DGTAYTGMLDILETYL

Cellulase 12A (Endo-beta-1,4-glucanase (EC 3.2.1.4)) (Endoglucanase S) (Glycoside hydrolase) Bacillus licheniformis

MKNNHLLKSILLWGAVCIIVLAGPLSAFAASSSNPSDKLYFKNKK
YYIFNNVWGADQVSGWWQTIYHNSDSDMGWVWNWPSNTSTVKAYP
SIVSGWHWTEGYTAGSGFPTRLSDQKNINTKVSYSISANGTYNAA
YDIWLHNTNKASWDSAPTDEIMIWLNNTNAGPAGSYVETVSIGGH
SWKVYKGYIDAGGGKGWNVFSFIRTANTQSANLNIRDFTNYLADS
KQWLSKTKYVSSVEFGTEVFGGTGQINISNWDVTVR

Cellulase (EC 3.2.1.4)Bacillus licheniformis (strain ATCC 14580/DSM 13/JCM 2505/CCUG 7422/NBRC 12200/NCIMB 9375/NCTC 10341/NRRL NRS-1264/Gibson 46)

MREKWLALCLKSIILFVLIMGTVLLSAVPKASGTSGPAKQEKQSI
ITPADLLENMSPGWNLGNTLDAVPTEGSWNNPPVREHTFDDIRDA
GFKSVRIPVTWDSHIGSAPEYPIDTDWMNRVEEVTDWALEREFYV
VLNIHHDSWLWISRMGNSQQETLDKLGKVWKQIAERFKNKSERLL
FEIVNEPTGMSAYQMNLLNREMLNIIRSTGGKNGQRLVIVGGLED
NKDELLHSFEPPDDDRIVLTYHYYSPWDYVSNWWGRTTWGSAAEI
SEMEEDIKPVYEKFVREGYPVIIGEYGTLGANEKHSKWLYHDTFV
RLAHKYQMVPMWWDNGNDQFDRAERQWRDPVVKEIVIQAGRGVPN
AIIKPADLFIKKGQSISDQTVDIQLNGNVLTGIYQKSEPLKEGSD
YTVDNAGKTVSIKASCLAKLLNGAGQPGVKAQLTFTFHKGASQVM
DIILYDDPKLEKSEFTISQSAISGDLKIPASLNGTKLATVKGVVD
STGRPVLEEVWSWTPYLNYDEDFYEKDGDLYLKERVLKYLKSDST
FTFELWPKGVEAVVKVKITP

Endoglucanase A (EC 3.2.1.4) (Endo-1,4-beta-glucanase A) Bacillus pumilus (Bacillus mesentericus)

MLIFETYLILFKTVQITKRRIERRRLRLLNQCFTKKEGVSNREMA
SYNYVEVLQKSMLFYEAQRSGRLPESNRLNWRGDSGLKDGKDVGH
DLTGGWYDAGDHVKFGLPMAYSAAVLAWTVYEYREAYEEAELLDE
ILDQIKWATDYFLKAHTGPNEFWAQVGDGNADHAWWGPAEVMPMN
RPAFKIDEHCPGTEVAAQTAAALAAGSIIFKETDASYAAKLLTHA
KQLYAFADRYRGKYTDCVTNAQPFYNSWSGYVDELIWGGIWLYLA
TNEETYLNKALKAVEEWPQDWDYTFTMSWDNTFFASQILLARITK
ENRFIESTERNLDYWTTGLVQNGKVERITYTPGGLAWLDQWGSLR
YAANAAFLAFVYADWVSDQEKKNRYQSFAIKQTHYMLGDNPLNRS
YVVGFGQNSPKHPHHRTAHGSWSNQLTNPPSHRHTLYGALVGGPN
AQDQYDDDISDYISNEVATDYNAAFTGNIAKMVQLFGEGQSKLPN
FPPKEQVEDEFFVEAAVMHNDTTSTQVKAVLYNRSGWPARSSQTL
SFRYYVNLSEVFAKGFTEKDIQVTAAYNEGASLSPLKVYDASSRV
YFAEIDFTGVVISPRGESEHKKEIQFRLSAPNGSNIWDASNDYSY
QGLTSNMQKTTKIPVFEDGVLVFGTLPDK

Endoglucanase A (EC 3.2.1.4) (Endo-1,4-beta-glucanase A) Bacillus pumilus (Bacillus mesentericus)

MLIFETYLILFKTVQITKRRIERRRLRLLNQCFTKKEGVSNREMA
SYNYVEVLQKSMLFYEAQRSGRLPESNRLNWRGDSGLKDGKDVGH
DLTGGWYDAGDHVKFGLPMAYSAAVLAWTVYEYREAYEEAELLDE
ILDQIKWATDYFLKAHTGPNEFWAQVGDGNADHAWWGPAEVMPMN
RPAFKIDEHCPGTEVAAQTAAALAAGSIIFKETDASYAAKLLTHA
KQLYAFADRYRGKYTDCVTNAQPFYNSWSGYVDELIWGGIWLYLA
TNEETYLNKALKAVEEWPQDWDYTFTMSWDNTFFASQILLARITK
ENRFIESTERNLDYWTTGLVQNGKVERITYTPGGLAWLDQWGSLR
YAANAAFLAFVYADWVSDQEKKNRYQSFAIKQTHYMLGDNPLNRS
YVVGFGQNSPKHPHHRTAHGSWSNQLTNPPSHRHTLYGALVGGPN
AQDQYDDDISDYISNEVATDYNAAFTGNIAKMVQLFGEGQSKLPN
FPPKEQVEDEFFVEAAVMHNDTTSTQVKAVLYNRSGWPARSSQTL
SFRYYVNLSEVFAKGFTEKDIQVTAAYNEGASLSPLKVYDASSRV
YFAEIDFTGVVISPRGESEHKKEIQFRLSAPNGSNIWDASNDYSY
QGLTSNMQKTTKIPVFEDGVLVFGTLPDK

Cellulase (EC 3.2.1.4)Bacillus sp

MKKITTIFVVLLMTVALFSIGNTTAADNDSVVEEHGQLSISNGEL
VNERGEQVQLKGMSSHGLQWYGQFVNYESMKWLRDDWGITVFRAA
MYTSSGGYIDDPSVKEKVKEAVEAAIDLDIYVIIDWHILSDNDPN
IYKEEAKDFFDEMSELYGDYPNVIYEIANEPNGSDVTWDNRIKPY
AEEVIPVIRNNDPNNIIIVGTGTWSQDVHHAADNQLADPNVMYAF
HFYAGTHGQNLRDQVDYALDQGAAIFVSEWGTSAATGDGGVFLDE
AQEWIDFMDERNLSWANWSLTHKDESSAALMPGANPTGGWTEAEL
SPSGTFVREKIRESASIPPSDPTPPSDPGEYPAWDPTQIYTNEIV
YHNGQLWQAKWWTQNQEPGDPYGPWEPLN

Endoglucanase (EC 3.2.1.4) (Cellulase) (Endo-1,4-beta-glucanase) (Endo-K) Bacillus sp. (strain KSM-330)

MVEKRKIFTVLCACGIGFTSYTSCISAAAIDNDTLINNGHKINSS
IITNSSQVSAVAKEMKPFPQQVNYSGILKPNHVSQESLNNAVKNY
YNDWKKKYLKNDLSSLPGGYYVKGEITGNPDGFRPLGTSEGQGYG
MIITVLMAGHDSNAQTIYDGLFKTARAFKSSINPNLMGWVVADDK
KAQGHFDSATDGDLDIAYSLLLAHKQWGSSGKINYLKEAQNMITK
GIKASNVTKNNGLNLGDWGDKSTFDTRPSDWMMSHLRAFYEFTGD
KTWLNVIDNLYNTYTNFTNKYSPKTGLISDFVVKNPPQPAPKDFL
DESKYTDSYYYNASRVPLRIVMDYAMYGEKRGKVISDKVATWIKS
KTKGNPSKIVDGYKLDGTNIGDYPTAVYVSPFIAAGTTNSKNQEW
VNSGWDWMKNKKESYFSDSYNLLTMLFLTGNWWKPIPDEKKIQSP
INLEVQSELKEQD

Endoglucanase (EC 3.2.1.4) (Alkaline cellulase) (Endo-1,4-beta-glucanase) Bacillus sp. (strain KSM-635)

MKIKQIKQSLSLLLIITLIMSLFVPMASANTNESKSNAFPFSDVK
KTSWSFPYIKDLYEQEVITGTSATTFSPTDSVTRAQFTVMLTRGL
GLEASSKDYPFKDRKNWAYKEIQAAYEAGIVTGKTNGEFAPNENI
TREQMAAMAVRAYEYLENELSLPEEQREYNDSSSISTFAQDAVQK
AYVLELMEGNTDGYFQPKRNSTREQSAKVISTLLWKVASHDYLYH
TEAVKSPSEAGALQLVELNGQLTLAGEDGTPVQLRGMSTHGLQWF
GEIVNENAFVALSNDWGSNMIRLAMYIGENGYATNPEVKDLVYEG
IELAFEHDMYVIVDWHVHAPGDPRADVYSGAYDFFEEIADHYKDH
PKNHYIIWELANEPSPNNNGGPGLTNDEKGWEAVKEYAEPIVEML
REKGDNMILVGNPNWSQRPDLSADNPIDAENIMYSVHFYTGSHGA
SHIGYPEGTPSSERSNVMANVRYALDNGVAVFATEWGTSQANGDG
GPYFDEADVWLNFLNKHNISWANWSLTNKNEISGAFTPFELGRTD
ATDLDPGANQVWAPEELSLSGEYVRARIKGIEYTPIDRTKFTKLV
WDFNDGTTQGFQVNGDSPNKESITLSNNNDALQIEGLNVSNDISE
GNYWDNVRLSADGWSENVDILGATELTIDVIVEEPTTVSIAAIPQ
GPAAGWANPTRAIKVTEDDFESFGDGYKALVTITSEDSPSLETIA
TSPEDNTMSNIILFVGTEDADVISLDNITVSGTEIEIEVIHDEKG
TATLPSTFEDGTRQGWDWHTESGVKTALTIEEANGSNALSWEYAY
PEVKPSDGWATAPRLDFWKDELVRGTSDYISFDFYIDAVRASEGA
ISINAVFQPPANGYWQEVPTTFEIDLTELDSATVTSDELYHYEVK
INIRDIEAITDDTELRNLLLIFADEDSDFAGRVFVDNVRFE

Cellulase (EC 3.2.1.4) Bacillus sp. BG-CS10

MVHIQRKFILTKSLIVVFTMLLCLSLMTPPLLADEGAKQTDIQSY
VADMQPGWNLGNTFDAVGDDETAWGNPRVTRELIKTIADEGYKSI
RIPVTWENQMGNAPDYTINEDFFSRVEQVIDWALEEDLYVMLNLH
HDSWLWIYNMEHNYDEVMAKYTALWEQLSERFQGHSHKLMFESVN
EPRFTRDWGEIQENHHAFLEELNTAFYHIVRESGGSNTERPLVLP
TLETATSQDLLNRLHQTMKDLNDPNLIATVHYYGFWPFSVNVAGY
TRFEEETQQDIIDTFNRVHNTFTANGIPVVLGEFGLLGFDTSTDV
IQQGEKLKFFEFLIHHLNERDVTHMLWDNGQHLNRETYSWYDQEF
HNMLKASWEGRSATAESNLIHVRDGEPIRDQDIQLHLHGNELTGL
QVDGDSLALGEDYELAGDVLTMKADALTALMTPGELGTNAVITAQ
FNSGADWHFQLQNVDEPTLENTEGSTSNFAIPAHFNGDSVATMEA
VYANGEFAGPQNWTSFKEFGYTFSPVYDKGEIVITDAFFNEVRDD
DIHLTFHFWSGEMVEYTLSKNGNHVQGRR

Endoglucanase (EC 3.2.1.4) (Carboxymethyl-cellulase) (CMCase) (Cellulase) (Endo-1,4-beta-glucanase) Bacillus subtilis (strain 168)

MKRSISIFITCLLITLLTMGGMIASPASAAGTKTPVAKNGQLSIK
GTQLVNRDGKAVQLKGISSHGLQWYGEYVNKDSLKWLRDDWGITV
FRAAMYTADGGYIDNPSVKNKVKEAVEAAKELGIYVIIDWHILND
GNPNQNKEKAKEFFKEMSSLYGNTPNVIYEIANEPNGDVNWKRDI
KPYAEEVISVIRKNDPDNIIIVGTGTWSQDVNDAADDQLKDANVM
YALHFYAGTHGQFLRDKANYALSKGAPIFVTEWGTSDASGNGGVF
LDQSREWLKYLDSKTISWVNWNLSDKQESSSALKPGASKTGGWRL
SDLSASGTFVRENILGTKDSTKDIPETPSKDKPTQENGISVQYRA
GDGSMNSNQIRPQLQIKNNGNTTVDLKDVTARYWYKAKNKGQNFD
CDYAQIGCGNVTHKFVTLHKPKQGADTYLELGFKNGTLAPGASTG
NIQLRLHNDDWSNYAQSGDYSFFKSNTFKTTKKITLYDQGKLIWG
TEPN

Endoglucanase (EC 3.2.1.4) (Carboxymethyl-cellulase) (CMCase) (Cellulase) (Endo-1,4-beta-glucanase) Bacillus subtilis (strain 168)

MKRSISIFITCLLITLLTMGGMIASPASAAGTKTPVAKNGQLSIKGTQLVNRDGKAVQ
LKGISSHGLQWYGEYVNKDSLKWLRDDWGITVFRAAMYTADGGYIDNPSVKNKVK
EAVEAAKELGIYVIIDWHILNDGNPNQNKEKAKEFFKEMSSLYGNTPNVIYEIANEPN
GDVNWKRDIKPYAEEVISVIRKNDPDNIIIVGTGTWSQDVNDAADDQLKDANVMYA
LHFYAGTHGQFLRDKANYALSKGAPIFVTEWGTSDASGNGGVFLDQSREWLKYLDS
KTISWVNWNLSDKQESSSALKPGASKTGGWRLSDLSASGTFVRENILGTKDSTKDIP
ETPSKDKPTQENGISVQYRAGDGSMNSNQIRPQLQIKNNGNTTVDLKDVTARYWYK
AKNKGQNFDCDYAQIGCGNVTHKFVTLHKPKQGADTYLELGFKNGTLAPGASTGNI
QLRLHNDDWSNYAQSGDYSFFKSNTFKTTKKITLYDQGKLIWGTEPN

Endoglucanase A (EC 3.2.1.4) (Cellulase A) (Endo-1,4-beta-glucanase A) (EgA) Butyrivibrio fibrisolvens

MVSKKQKFLTVILVIVLAIVIVGGVFGISFVKGRVTFPWQLQNSEAKTEQVKEPAKEE
PKLVIKEKKQDESAKKEQELKKAKEEAEAAVEKETEKTEEEPVDNLLNDMKLKYYG
KLAVEGSHLVDADGHEVLLMGVSTHGINWYPEYASAETIKSLRDTWGINVIRLAMY
TSDYNGYCVAGKENQEKLKDIIDDAVEAATDNDMYVIIDWHTLNDADPNEYKADAI
QFFGEMVRKYKDNENVIYEICNEPNGDTTWNDVRRYANEVIPVIRNVDAIILVGTPK
WATDLDSVLDKPLDFDNIMYTYHFYAGTHHKAERNALRDALDEGLPVFISEYGLVD
ADGDGNLNEKEADYWYDMIRKEYGVSSCMWNLSNKDEGSAMINADCDKLSDFTE
EDLSESAMWLIDQISQLKHSDLEQGVDWITPENNNR

Cellodextrinase (EC 3.2.1.4) Butyrivibrio fibrisolvens

MKKVLVNQVGFLCNAPKKAVLNFQANEFSVVDGNGKKAFDGKVEHFGTDEISGED
TYVADFSALTEEGKYKIVADGQESVLFSISNDAYDKLMKDICKCFYYLRCGDALSKE
FAGEYYHKPCHMTKATVYGEDVEPVDVTGGWHDAGDYGRYSTAGAVAVAHLLY
GVRFFKGLLDVHYDIPKVAGDKGNLPEILAEVKVELDFLMKMQRENGSVWHKVTT
FNHAPFLMPEDDREELFLFSVSSLATADIAAVFALAYTVYKEYDAEYADKLMQKSLL
AYKWLLDNPDELLFFNPDGSNTGQYDEAEDISNRFWAACALYEATSDGKYYSDAQE
LKNRLEEFDKNAQKKGYQGNVFTCLGWAEVAGLGSLSLLLKREENALCSLARNSFV
AEADRLVKVSKENGFGLCMGENDFIWGSNMELLKYMMVLSTAIRIDNKPEYKLALE
AGLDYILGCNSMDISYVTGNGEKAFKNPHLRPTAVDDIEEPWPGLVSGGPNSGLHDE
RAQTLRGKGLPPMKCYIDHIDCYSLNEITIYWNSPLVFALSGILE

Endoglucanase A (EC 3.2.1.4) (Cellulase A) (Endo-1,4-beta-glucanase A) (EgA) Butyrivibrio fibrisolvens

MVSKKQKFLTVILVIVLAIVIVGGVFGISFVKGRVTFPWQLQNSEAKTEQVKEPAKEE
PKLVIKEKKQDESAKKEQELKKAKEEAEAAVEKETEKTEEEPVDNLLNDMKLKYYG
KLAVEGSHLVDADGHEVLLMGVSTHGINWYPEYASAETIKSLRDTWGINVIRLAMY
TSDYNGYCVAGKENQEKLKDIIDDAVEAATDNDMYVIIDWHTLNDADPNEYKADAI
QFFGEMVRKYKDNENVIYEICNEPNGDTTWNDVRRYANEVIPVIRNVDAIILVGTPK
WATDLDSVLDKPLDFDNIMYTYHFYAGTHHKAERNALRDALDEGLPVFISEYGLVD
ADGDGNLNEKEADYWYDMIRKEYGVSSCMWNLSNKDEGSAMINADCDKLSDFTE
EDLSESAMWLIDQISQLKHSDLEQGVDWITPENNNR

Endoglucanase (EC 3.2.1.4) Butyrivibrio proteoclasticus (strain ATCC 51982/DSM 14932/B316) (Clostridium proteoclasticum)

MHKSKCIKRVFTFLLALFVFVMAIPATKVSATGGTDRSATQVVSDMRVGWNIGNSL
DSFGQSYNFPYTSLNETYWGNPATTKALIDEVAKAGENTIRIPVSWGQYTSGSDYQIP
DFVMNRVKEVVDYCIVNDMYVILNSHHDINSDYCFYVPNNANKDRSEKYFKSIWTQ
IAKEFRNYDYHLVFETMNEPRLVGHGEEWWFPRNNPSNDIREAVACINDYNQVALD
AIRATGGNNATRCVMVPGYDASIEGCMTDGFKMPNDTASGRLILSVHAYIPYYFAL
ASDTYVTRFDDNLKYDIDSFFNDLNSKFLSRNIPVVVGETSATNRNNTAERVKWAD
YYWGRAARYSNVAMVLWDNNIYQNNSAGSDGECHMYIDRNSLQWKDPEIISTIMK
HVDGTPATINGKEIPSTEQPDPTPVDPDPTPVDPDPTPVDPDPTPVDPDPQPVDPTPVS
GALKAEYTINNWGSGYQVLIKVKNDSASRVDGWTLKISKSEVKIDSSWCVNIAEEG
GYYVITPMSWNSSLEPSASVDFGIQGSGSIGTSVNISVQ

Cellulase (EC 3.2.1.4)Caldicellulosiruptor saccharolyticus (strain ATCC 43494/DSM 8903/Tp8T 6331)

MKKECLRVFAVFMVFTFLLSLFPFVTFAQNTAYEKDKYPHLIGNSLVKKPSVAGRLQ
IIKQNGRRILADQNGEPIQLRGMSTHGLQWFPQIINNNAFAALANDWGCNVIRLAMY
IGEGGYATNPQVKDKVIEGIKLAIQNDMYVIVDWHVLNPGDPNAEIYKGAKDFFKEI
AQKFPNDFHIIYELCNEPNPTDPGVTNDEAGWKKVKAYAEPIIKMLRQMGNENIIIIG
SPNWSQRPDFAIKDPIADDKVMYSVHFYTGTHKVDGYVFENMKMAIEAGVPVFVTE
WGTSEASGDGGPYLDEADKWLEYLNANNISWVNWSLTNKNETSGAFVPYISGVSQ
ATDLDPGSDQKWDISELSISGEYVRSRIKGIPYQPIERTLKISQDQVACAPIGQPILPSD
FEDGTRQGWDWDGPSGVKGALTIEEANGSNALSWEVEYPEKKLQDGWASAPRLIL
RNINTTRGDCKYLCFDFYLKPKQATKGELAIFLAFAPPSLNYWAQAEDSFNIDLTNLS
TLKKTPDDLYSFKISFDLDKIKEGKIIGPDTHLRDIIIVVADVNSDFKGRMYLDNVRFT
NMLFEDVTPQTTGYEAISKLYSKKIVNGISTNLFGPEKAVTRAEVAAMAVRLLDLQE
ESYNGEFVDVSKNSWYANEVSTAYKAGIILGDGKYIKPEKAVTREEMAVFAMRIYRI
LTDEKAEATEEIAISDKNSISSWARQDVNAAISLGLMDVFTDGSFGPKVKVTRAEAT
QIIYKILELTGKM

Endoglucanase A (EC 3.2.1.4) (Cellulase A) (Endo-1,4-beta-glucanase A) Cellulomonas fimi

MSTRRTAAALLAAAAVAVGGLTALTTTAAQAAPGCRVDYAVTNQWPGGFGANVTI
TNLGDPVSSWKLDWTYTAGQRIQQLWNGTASTNGGQVSVTSLPWNGSIPTGGTASF
GFNGSWAGSNPTPASFSLNGTTCTGTVPTTSPTPTPTPTTPTPTPTPTPTPTPTVTPQPT
SGFYVDPTTQGYRAWQAASGTDKALLEKIALTPQAYWVGNWADASHAQAEVADY
TGRAVAAGKTPMLVVYAIPGRDCGSHSGGGVSESEYARWVDTVAQGIKGNPIVILEP
DALAQLGDCSGQGDRVGFLKYAAKSLTLKGARVYIDAGHAKWLSVDTPVNRLNQV
GFEYAVGFALNTSNYQTTADSKAYGQQISQRLGGKKFVIDTSRNGNGSNGEWCNPR
GRALGERPVAVNDGSGLDALLWVKLPGESDGACNGGPAAGQWWQEIALEMARNA
RW

Endoglucanase A (EC 3.2.1.4) (Cellulase A) (Endo-1,4-beta-glucanase A) Cellulomonas fimi

MSTRRTAAALLAAAAVAVGGLTALTTTAAQAAPGCRVDYAVTNQWPGGFGANVTI
TNLGDPVSSWKLDWTYTAGQRIQQLWNGTASTNGGQVSVTSLPWNGSIPTGGTASF
GFNGSWAGSNPTPASFSLNGTTCTGTVPTTSPTPTPTPTTPTPTPTPTPTPTPTVTPQPT
SGFYVDPTTQGYRAWQAASGTDKALLEKIALTPQAYWVGNWADASHAQAEVADY
TGRAVAAGKTPMLVVYAIPGRDCGSHSGGGVSESEYARWVDTVAQGIKGNPIVILEP
DALAQLGDCSGQGDRVGFLKYAAKSLTLKGARVYIDAGHAKWLSVDTPVNRLNQV
GFEYAVGFALNTSNYQTTADSKAYGQQISQRLGGKKFVIDTSRNGNGSNGEWCNPR
GRALGERPVAVNDGSGLDALLWVKLPGESDGACNGGPAAGQWWQEIALEMARNA
RW

Endoglucanase (EC 3.2.1.4) (Cellulase) (Endo-1,4-beta-glucanase) Cellulomonas uda

MPLRALVAVIVTTAVMLVPRAWAQTAWERYKARFMMPDARIIDTANGNVSHTEGQ
GFAMLLAVANNDRPAFDKLWQWTDSTLRDKSNGLFYWRYNPVAPDPIADKNNAT
DGDTLIAWALLRAQKQWQDKRYATASDAITASLLKYTVVTFAGRQVMLPGVKGFN
RNDHLNLNPSYFIFPAWRAFAERTHLTAWRTLQSDGQALLGQMGWGKSHLPSDWV
ALRADGKMLPAKEWPPRMSFDAIRIPLYISWVDPHSALLAPWKAWMQSYPRLQTPA
WINVSTNEVAPWNMAGGLLAVRDLTLGEPLERRRLTTRMIITPPASSCWSGWRNRIS
ASAVMALQVSQPVCLRAERKEQERLTM

Endoglucanase B (EGB) (EC 3.2.1.4) (Cellulase) (Endo-1,4-beta-glucanase) Cellvibrio japonicus (strain Ueda107) (Pseudomonas fluorescens subsp. cellulosa)

MNLLSGWVRPLMLGCGLLGAALSAGSIQAAVCEYRVTNEWGSGFTASIRITNNGSST
INGWSVSWNYTDGSRVTSSWNAGLSGANPYSATPVGWNTSIPIGSSVEFGVQGNNG
SSRAQVPAVTGAICGGQGSSAPSSVASSSSSSSVVSSTPRSSSSSVSSSVPGTSSSSSSSV
LTGAQACNWYGTLTPLCNNTSNGWGYEDGRSCVARTTCSAQPAPYGIVSTSSSTPLS
SSSSSRSSVASSSSLSSATSSSASSVSSVPPIDGGCNGYATRYWDCCKPHCGWSANVP
SLVSPLQSCSANNTRLSDVSVGSSCDGGGGYMCWDKIPFAVSPTLAYGYAATSSGD
VCGRCYQLQFTGSSYNAPGDPGSAALAGKTMIVQATNIGYDVSGGQFDILVPGGGV
GAFNACSAQWGVSNAELGAQYGGFLAACKQQLGYNASLSQYKSCVLNRCDSVFGS
RGLTQLQQGCTWFAEWFEAADNPSLKYKEVPCPAELTTRSGMNRSILNDIRNTCP

Endoglucanase C (EC 3.2.1.4) (Cellodextrinase C) (Cellulase C) (Endo-1,4-beta-glucanase C) (EGC) Cellvibrio japonicus (strain Ueda107) (Pseudomonas fluorescens subsp. cellulosa)

MGHVTSPSKRYPASFKRAGSILGVSIALAAFSNVAAAGCEYVVTNSWGSGFTAAIRIT
NSTSSVINGWNVSWQYNSNRVTNLWNANLSGSNPYSASNLSWNGTIQPGQTVEFGF
QGVTNSGTVESPTVNGAACTGGTSSSVSSSSVVSSSSSSRSSVSSSSVVSSSSSVVSSSS
SSVVSGGQCNWYGTLYPLCVSTTSGWGYENNRSCISPSTCSAQPAPYGIVGGSSSPSS
ISSSSVRSSSSSSVVPPSSSSSSSVPSSSSSSVSSSSVVSSSSSSVSVPGTGVFRVNTQGNL
TKDGQLLPARCGNWFGLEGRHEPSNDADNPSGAPMELYAGNMWWVNNSQGSGRT
IQQTMTELKQQGITMLRLPIAPQTLDANDPQGRSPNLKNHQSIRQSNARQALEDFIKL
ADQNDIQIFIDIHSCSNYVGWRAGRLDARPPYVDANRVGYDFTREEYSCSATNNPSS
VTRIHAYDKQKWLANLREIAGLSAKLGVSNLIGIDVFNEPYDYTWAEWKGMVEEAY
QAINEVNPNMLIIVEGISANANTQDGTPDTSVPVPHGSTDLNPNWGENLYEAGANPP
NIPKDRLLFSPHTYGPSVFVQRQFMDPAQTECAGLEGDEAAQARCRIVINPTVLEQG
WEEHFGYLRELGYGILIGEFGGNMDWPGAKSSQADRNAWSHITTNVDQQWQQAAA
SYFKRKGINACYWSMNPESADTMGWYLTPWDPVTANDMWGQWTGFDPRKTQLLH
NMWGL

Cellulase, putative, cel5D (EC 3.2.1.4) Cellvibrio japonicus (strain Ueda107) (Pseudomonas fluorescens subsp. cellulosa)

MPHRGQGCIRNWLLTIKPRSIMNRINTLVFSCLLVVLAGCGSAGGGSSGGASSSIITPS
SSDTSSLSSSASSTSSLSSASSLSSSSSSSSSLSSAGLYPSYNTSPAAPDSTGMQSTAVQL
AGKIRLGWNIGNTMEAIGGETAWGNPMVSNELLKLVKDSGFDAVRIPVAWDQYAN
QESAEISAAWLNRVKQVVQMAIDNELYVLINIHWDGGWLENNITPAKKDENNAKQ
KAFWEQIATHLRDFDEHLLFAGTNEPNAENAEQMDVLNSYLQTFVDAVRSTGGKN
AYRVLVLQGPVTDIEKTNELWTHMPADTATDRLMAEVHFYTPYNFALMRQDESWG
KQFYYWGEGFLSTTDTERNPTWGEEATIDQLFDLMKTKFVDQGIPVVLGEFSAMRR
TNLTGDALTLHLAGRAYYHKYVTQQALARGLLPFYWDNGGNDNFSSGIFNRQQNT
VFDQQVLDALLEGAGAQ

Endoglucanase B (EGB) (EC 3.2.1.4) (Cellulase) (Endo-1,4-beta-glucanase) Cellvibrio japonicus (strain Ueda107) (Pseudomonas fluorescens subsp. cellulosa)

MNLLSGWVRPLMLGCGLLGAALSAGSIQAAVCEYRVTNEWGSGFTASIRITNNGSST
INGWSVSWNYTDGSRVTSSWNAGLSGANPYSATPVGWNTSIPIGSSVEFGVQGNNG
SSRAQVPAVTGAICGGQGSSAPSSVASSSSSSSVVSSTPRSSSSSVSSSVPGTSSSSSSSV
LTGAQACNWYGTLTPLCNNTSNGWGYEDGRSCVARTTCSAQPAPYGIVSTSSSTPLS
SSSSSRSSVASSSSLSSATSSSASSVSSVPPIDGGCNGYATRYWDCCKPHCGWSANVP
SLVSPLQSCSANNTRLSDVSVGSSCDGGGGYMCWDKIPFAVSPTLAYGYAATSSGD
VCGRCYQLQFTGSSYNAPGDPGSAALAGKTMIVQATNIGYDVSGGQFDILVPGGGV
GAFNACSAQWGVSNAELGAQYGGFLAACKQQLGYNASLSQYKSCVLNRCDSVFGS
RGLTQLQQGCTWFAEWFEAADNPSLKYKEVPCPAELTTRSGMNRSILNDIRNTCP

Cellulase (EC 3.2.1.4)Clavibacter michiganensis

MRKASVEVVFSAAGWLPYDPYMTFRQVRASLVLRLVLLLALVVGTTSAAFAAPVS
AATVAGPVAAASSPGWLHTAGGKIVTASGAPYTIRGIAWFGMETSSCAPHGLDTITL
AGGMQHIKQMGFTTVRLPFSNQCLAASGVTGVSADPSLAGLTPLQVMDHVVASAK
SAGLDVILDQHRPDSGGQSELWYTSQYPESRWISDWRMLAKRYAAEPTVIGVDLHN
EPHGAATWGTGAATTDWRAAAERGGNAVLAENPNLLVLVEGIDHEADGSGTWWG
GALGLVGNAPVRLSVANRVVYSPHDYPSTIYGQSWFSASNYPANLPGIWDAHWGYL
AKKDIAPVLVGEFGTKFETTSDKQWLNTLVGYLSSTGISSSFWAFNPNSGDTGGIVKS
DWVTPEQAKLDALAPILHPSPGSGPGSGGSGSQPAPQPDPANPGAVSAKWQPGSSW
ASGYVANIDVTATAAVTGWTVSWASPGTTRVVNSWGMRCSVASGTVSCTGTDWA
SKLAAGQTVHVGLQASGGPAPSSPRLTATAAAVPPAQPTPPARPTTHGRATHYSLGQ
GNTIANGNCSMPAVPADRMYVAVSSPEYSGAAACGTFLDVTGPKGTVRVQVADQC
HGCEVGHLDLSEEAFRALGDFNAGIIPISYVTVRDPAGPTVAIRVKEGSSRWWAGLQ
VLNAGNRIDRVEIQAGRQWLPLTRTDYGYWVTPSPIQDGPLTVKVTDQYGRAVVLP
GLRMAPGEIQRTASRFYPV

Cellulase (EC 3.2.1.4)Clavibacter michiganensis subsp. michiganensis (strain NCPPB 382)

MRKASVEVVFSAAGWLPYDPYMTFRQVRASLVLRLVLLLALVVGTTSAAFAAPVS
AATVAGPVAAASSPGWLHTAGGKIVTASGAPYTIRGIAWFGMETSSCAPHGLDTITL
AGGMQHIKQMGFTTVRLPFSNQCLAASGVTGVSADPSLAGLTPLQVMDHVVASAK
SAGLDVILDQHRPDSGGQSELWYTSQYPESRWISDWRMLAKRYAAEPTVIGVDLHN
EPHGAATWGTGAATTDWRAAAERGGNAVLAENPNLLVLVEGIDHEADGSGTWWG
GALGLVGNAPVRLSVANRVVYSPHDYPSTIYGQSWFSASNYPANLPGIWDAHWGYL
AKKDIAPVLVGEFGTKFETTSDKQWLNTLVGYLSSTGISSSFWAFNPNSGDTGGIVKS
DWVTPEQAKLDALAPILHPSPGSGPGSGGSGSQPAPQPDPANPGAVSAKWQPGSSW
ASGYVANIDVTATAAVTGWTVSWASPGTTRVVNSWGMRCSVASGTVSCTGTDWA
SKLAAGQTVHVGLQASGGPAPSSPRLTATAAAVPPAQPTPPARPTTHGRATHYSLGQ
GNTIANGNCSMPAVPADRMYVAVSSPEYSGAAACGTFLDVTGPKGTVRVQVADQC
HGCEVGHLDLSEEAFRALGDFNAGIIPISYVTVRDPAGPTVAIRVKEGSSRWWAGLQ
VLNAGNRIDRVEIQAGRQWLPLTRTDYGYWVTPSPIQDGPLTVKVTDQYGRAVVLP
GLRMAPGEIQRTASRFYPVH

Endo-1,4-beta glucanase EngF (EC 3.2.1.4) Clostridium cellulovorans

MFNNVKKKILSIVAAGAMLMALVPNINVAAETTYSNLTGNANVKKPSVGGKLQLLN
KNGIKTLCDKDGNPIQLRGMSTHGLQWFPGVVNNNAFAALSNDWNSNVIRLAMYV
AEGGYATNPSVKQTVINGINYAIANDMYVIVDWHMMNPGDPNASVYSGAQSFENDI
STLYPNNKNIIYELCNEPNGENGGVTNDATGWAQVKSYATPIVQLLRNKGNENLIIV
GNPFWSQRPDLAADNPINDSNTMYSVHFYSGTNPISTVDTNRDNAMSNVRYALNHG
AAVFATEWGTSLATGTTGPYLAKADAWLDFLNGNNISWCNFSISNKDEKAAALNSL
TSLDPGSDKLWADNELTTSGQYVRARIKGAYYATPVDPVTNQPTAPKDFSSGFWDF
NDGTTQGFGVNPDSPITAINVENANNALKISNLNSKGSNDLSEGNFWANVRISADIW
GQSINIYGDTKLTMDVIAPTPVNVSIAAIPQSSTHGWGNPTRAIRVWTNNFVAQTDGT
YKATLTISTNDSPNFNTIATDAADSVVTNMILFVGSNSDNISLDNIKFTK

Endoglucanase D (EC 3.2.1.4) (Cellulase D) (Endo-1,4-beta-glucanase D) Clostridium cellulovorans (strain ATCC 35296/DSM 3052/OCM 3/743B)

MIKHLLSRGKLLLFVSVMATSSIIAGGNAYGSTAFTGVRDVPAQQIVNEMKVGWNL
GNTMDAIGGETNWGNPMTTHAMINKIKEAGFNTLRLPVTWDGHMGAAPEYTIDQT
WMKRVEEIANYAFDNDMYVIINLHHENEWLKPFYANEAQVKAQLTKVWTQIANNF
KKYGDHLIFETMNEPRPVGASNEWTGGSYENREVVNRYNLTAVNAIRATGGNNAT
RYIMVPTLAASAMSTTINDLVIPNNDSKVIVSLHMYSPYFFAMDINGTSSWGSDYDK
SSLDSEFDAVYNKFVKNGRAVVIGEMGSINKNNTAARVTHAEYYAKSAKARGLTPI
WWDNGYSVAGKAETFGIFNRSNLTWDAPEVMKAFIKGIGGSSTTTPTTPTTPTTPTTP
TTPTTPTTPTTPTTPQSAVEVTYAITNSWGSGASVNVTIKNNGTTPINGWTLKWTMPI
NQTITNMWSASFVASGTTLSVTNAGYNGTIAANGGTQSFGFNINYSGVLSKPTGFTV
NGTECTVK

Endoglucanase D (EC 3.2.1.4) (Cellulase D) (Endo-1,4-beta-glucanase D) Clostridium cellulovorans (strain ATCC 35296/DSM 3052/OCM 3/743B)

MIKHLLSRGKLLLFVSVMATSSIIAGGNAYGSTAFTGVRDVPAQQIVNEMKVGWNL
GNTMDAIGGETNWGNPMTTHAMINKIKEAGFNTLRLPVTWDGHMGAAPEYTIDQT
WMKRVEEIANYAFDNDMYVIINLHHENEWLKPFYANEAQVKAQLTKVWTQIANNF
KKYGDHLIFETMNEPRPVGASNEWTGGSYENREVVNRYNLTAVNAIRATGGNNAT
RYIMVPTLAASAMSTTINDLVIPNNDSKVIVSLHMYSPYFFAMDINGTSSWGSDYDK
SSLDSEFDAVYNKFVKNGRAVVIGEMGSINKNNTAARVTHAEYYAKSAKARGLTPI
WWDNGYSVAGKAETFGIFNRSNLTWDAPEVMKAFIKGIGGSSTTTPTTPTTPTTPTTP
TTPTTPTTPTTPTTPQSAVEVTYAITNSWGSGASVNVTIKNNGTTPINGWTLKWTMPI
NQTITNMWSASFVASGTTLSVTNAGYNGTIAANGGTQSFGFNINYSGVLSKPTGFTV
NGTECTVK

Endoglucanase A (EC 3.2.1.4) (Cellulase A) (Endo-1,4-beta-glucanase A) Clostridium longisporum

MKRSLLKTCSIIAGATIIFSSLSISRNPLEVQAASMRSASEIVQEMGVGWNLGNTLDA
KITNLSYNTSPISFETGWGNPVTTKAMIDKIKNAGFKTIRIPTTWGEHLDGNNKLNEE
WVKRVKEVVDYCIADDLYVILNTHHEGNWVIPTYAKESSVTPKLKTLWTQISEAFK
DYDDHLIFETLNEPRLEGTPYEWTGGTSESRDVVNKYNAAALESIRKTGGNNLSRAV
MMPTYAASGSSTTMNDFKVPDDKNVIASVHAYSPYFFAMDTSSNSVNTWGSSYDK
YSLDVELDSYLNTFKSKGVPVVIGEFGSINKNNTSSRAELAEYYVTAAQKRGIPCVW
WDNNYAETNKGETFGLLNRSTLNWYFSDIKDALIRGYKNVHPEATEDDKPSTDVTN
PDSGNTKPDSGNTNPGTETTTPTDNEKISITSKINDWGGAYQADFTLKNNTSSDINNW
SFKIKKNDIVFTNYWDVKITEENGYYVVTPQAWKTTILANSSIVISIQGTGKVISNFEY
KFD

Endoglucanase A (EC 3.2.1.4) (Cellulase A) (Endo-1,4-beta-glucanase A) Clostridium longisporum

MKRSLLKTCSIIAGATIIFSSLSISRNPLEVQAASMRSASEIVQEMGVGWNLGNTLDA
KITNLSYNTSPISFETGWGNPVTTKAMIDKIKNAGFKTIRIPTTWGEHLDGNNKLNEE
WVKRVKEVVDYCIADDLYVILNTHHEGNWVIPTYAKESSVTPKLKTLWTQISEAFK
DYDDHLIFETLNEPRLEGTPYEWTGGTSESRDVVNKYNAAALESIRKTGGNNLSRAV
MMPTYAASGSSTTMNDFKVPDDKNVIASVHAYSPYFFAMDTSSNSVNTWGSSYDK
YSLDVELDSYLNTFKSKGVPVVIGEFGSINKNNTSSRAELAEYYVTAAQKRGIPCVW
WDNNYAETNKGETFGLLNRSTLNWYFSDIKDALIRGYKNVHPEATEDDKPSTDVTN
PDSGNTKPDSGNTNPGTETTTPTDNEKISITSKINDWGGAYQADFTLKNNTSSDINNW
SFKIKKNDIVFTNYWDVKITEENGYYVVTPQAWKTTILANSSIVISIQGTGKVISNFEY
KFD

Endoglucanase (EC 3.2.1.4) Clostridium sp

MRKRGLAVVVALVLAATPVMGTLQSNLVVKADQQTNTNLLANGNFNSGLSDWDY
FTAEGGKGSVASSNGKLVATVSENGTENYSMQVYHEGFQMYKGGVYELSFEISSSV
ARDVDYRIQLNGGDYRGYYDGRVKTNSTTQKVDVKFTMKEENDIVPRLCFNIGNCG
ASLPSHQVTLDNVELKLLDGSQINYTSNEKKEQDININQVGYRTADQKQAIFRGSNV
GKSFSVVNTATNKTVYTGTVSASKNSSIANETNYIGDFSSVTAPGTYKITATGLADSY
EFTISDDVYTDAFTDLMRFFYLQRCEEIPSSLGGDFAHGECHTTKARILGTNEYIDTTG
GWHDAGDYGRYVVATSKAVTDILLAYQDNKDIFTDNTNIPESGNGQADVLDELKG
QFEWMLKMQNKNNGGVYHKVTCADFPSYVSADKEKAELIVCPISITATADFAACM
ALSYDTYKTTDATFAKTCLDAAKKAWSYLSTNSSGGFTNPSGINTGEYGDRNDSDE
RYFAAAALYYATGESTYHEAFKNMVNQGVKVGSDWATVGEYGNVLYLKSKNQDA
STKSKIESKILSEASDFLRVSQNEPYGISTGNNFIWGSNMVVADNAKLLMDAYNISGK
SEYKTAAIEHVNYIFGKNPMGMSYVTGYGTVSPKNPHHRPSMVAGKAIKGAMVGG
PDSALEDSIAKAYVSNAAPAKCYVDHSESYSTNEVDIYWNSAIVIALAQTGLVGKAS
QSTVPSTEPSVKPSETPSTEPSIVPSETPSTEPSVVPSETPSTEPSTVPSVKPSQPAGNTN
GIKVTHTVSGSDSTNQVYQIQNSGSSAIDLSKLVITYKFTKNDSKNMVLWCDNAAAQ
LNVAPYYASISSGVKTSVTKSGSEFTVKISFAEAFSLAPNAGYVQLQTRLTNEDWSSV
SGLKETGMQVTYGGTEI

Endoglucanase C307 (EC 3.2.1.4) (Cellulase C307) (Endo-1,4-beta-glucanase C307) Clostridium sp. (strain F1)

MVSFKAGINLGGWISQYQVFSKEHFDTFITEKDIETIAEAGFDHVRLPFDYPIIESDDN
VGEYKEDGLSYIDRCLEWCKKYNLGLVLDMHHAPGYRFQDFKTSTLFEDPNQQKRF
VDIWRFLAKRYINEREHIAFELLNEVVEPDSTRWNKLMLECVKAIREIDSTRWLYIGG
NNYNSPDELKNLADIDDDYIVYNFHFYNPFFFTHQKAHWSESAMAYNRTVKYPGQY
EGIEEFVKNNPKYSFMMELNNLKLNKELLRKDLKPAIEFREKKKCKLYCGEFGVIAI
ADLESRIKWHEDYISLLEEYDIGGAVWNYKKMDFEIYNEDRKPVSQELVNILARRKT

cellulase (EC 3.2.1.4) Cupriavidus necator (strain ATCC 43291/DSM 13513/CCUG 52238/LMG 8453/N-1) (Ralstonia eutropha)

MLRTRILHMIRPAPLLVAVTAHCLPPRGWRHGATGADMRRTLTGLTRWLLAGALA
CASLAAPAASAPVCKWADWDAFRQTLLSADGRVIDASTENQATVSEGQAYGLLFAL
AANDRASFDKLLAWTENNLAQGDLIAHLPAWIWGRLPPEQARSAGKAADSWGVID
ANPASDADLWLAYTLLEAGRLWQERRFTALGTLMARRILREETAVLPGLGRTVLPG
TQGFKLSERRWRLNPSYVPMQVMRRLATLLPDEPAWQQLVASSARLMLDTAPRGFS
PDWVEYETGRGFLPDAQTQAESAYNAIRVYLWAGMLASDAPQRAALLRAFTPLAD
YVAAKGFPPERVDTQTGQPGYYTQVLTLFGLGYLDGQFRFARDGALVPAWETGCP
AR

Endoglucanase Z (EC 3.2.1.4) (Cellulase Z) (Endo-1,4-beta-glucanase Z) (EGZ) Dickeya dadantii (strain 3937) (Erwinia chrysanthemi (strain 3937))

MPLSYLDKNPVIDSKKHALRKKLFLSCAYFGLSLACLSSNAWASVEPLSVNGNKIYA
GEKAKSFAGNSLFWSNNGWGGEKFYTADTVASLKKDWKSSIVRAAMGVQESGGYL
QDPAGNKAKVERVVDAAIANDMYAIIGWHSHSAENNRSEAIRFFQEMARKYGNKP
NVIYEIYNEPLQVSWSNTIKPYAEAVISAIRAIDPDNLIIVGTPSWSQNVDEASRDPINA
KNIAYTLHFYAGTHGESLRNKARQALNNGIALFVTEWGTVNADGNGGVNQTETDA
WVTFMRDNNISNANWALNDKNEGASTYYPDSKNLTESGKKVKSIIQSWPYKAGSA
ASATTDPSTDTTTDTTVDEPTTTDTPATADCANANVYPNWVSKDWAGGQPTHNEA
GQSIVYKGNLYTANWYTASVPGSDSSWTQVGSCN

Minor endoglucanase Y (EC 3.2.1.4) (Cellulase Y) (Endo-1,4-beta-glucanase Y) (EGY) Dickeya dadantii (strain 3937) (Erwinia chrysanthemi (strain 3937))

MGKPMWRCWALMLMVWFSASATAANGWEIYKSRFMTTDGRIQDTGNKNVSHTEG
QGFAMLMAVHYDDRIAFDNLWNWTQSHLRNTTSGLFYWRYDPSAANPVVDKNNA
SDGDVLIAWALLKAGNKWQDNRYLQASDSIQKAIIASNIIQFAGRTVMLPGAYGENK
NSYVILNPSYFLFPAWRDFANRSHLQVWRQLIDDSLSLVGEMRFGQVGLPTDWAAL
NADGSMAPATAWPSRFSYDAIRIPLYLYWYDAKTTALVPFQLYWRNYPRLTTPAWV
DVLSSNTATYNMQGGLLAVRDLTMGNLDGLSDLPGASEDYYSSSLRLLVMLARGK

Endoglucanase Z (EC 3.2.1.4) (Cellulase Z) (Endo-1,4-beta-glucanase Z) (EGZ) Dickeya dadantii (strain 3937) (Erwinia chrysanthemi (strain 3937))

MPLSYLDKNPVIDSKKHALRKKLFLSCAYFGLSLACLSSNAWASVEPLSVNGNKIYA
GEKAKSFAGNSLFWSNNGWGGEKFYTADTVASLKKDWKSSIVRAAMGVQESGGYL
QDPAGNKAKVERVVDAAIANDMYAIIGWHSHSAENNRSEAIRFFQEMARKYGNKP
NVIYEIYNEPLQVSWSNTIKPYAEAVISAIRAIDPDNLIIVGTPSWSQNVDEASRDPINA
KNIAYTLHFYAGTHGESLRNKARQALNNGIALFVTEWGTVNADGNGGVNQTETDA
WVTFMRDNNISNANWALNDKNEGASTYYPDSKNLTESGKKVKSIIQSWPYKAGSA
ASATTDPSTDTTTDTTVDEPTTTDTPATADCANANVYPNWVSKDWAGGQPTHNEA
GQSIVYKGNLYTANWYTASVPGSDSSWTQVGSCN

Glucanase (EC 3.2.1.→Enterobacter sp. CJF-002

MKALRWVVMALVLAAMNVRAACTWPAWEHFKRAYISDGGRVIDPSDARKITTSEG
QSYALFFALAADDRPMFDNVLEWTKDNLAQGDPGEHLPAWLWGKKDENNWTVLD
SNSASDADIWIAWSLLEAGRLWKEARYTTLGNALLNRIAKEEVVTVPGLGPMLLPG
KVGFAEETVWRLNPSYLPPQIARYLTRFGEPWTTLQETNHRLLLETAPKGFSPDWVR
YEKSKGWQLAPDKTLISGYDAIRVYLWVGMMNDHDAQKASLLERLKPMAALTAK
KGVVPEKVDVATAQPRGDGPVGFAAALLPFLQDRDAQAVQRQKVADHFPGDDAYF
SYVLTLFGQGWDEHRFRFTPRGELQPDWGQACASSQ

Endoglucanase (EC 3.2.1.4) (Carboxymethylcellulase) (CMCase) (Cellulase) (Endo-1,4-beta-glucanase) Escherichia coli (strain K12)

MNVLRSGIVTMLLLAAFSVQAACTWPAWEQFKKDYISQEGRVIDPSDARKITTSEGQ
SYGMFSALAANDRAAFDNILDWTQNNLAQGSLKERLPAWLWGKKENSKWEVLDS
NSASDGDVWMAWSLLEAGRLWKEQRYTDIGSALLKRIAREEVVTVPGLGSMLLPG
KVGFAEDNSWRFNPSYLPPTLAQYFTRFGAPWTTLRETNQRLLLETAPKGFSPDWVR
YEKDKGWQLKAEKTLISSYDAIRVYMWVGMMPDSDPQKARMLNRFKPMATFTEK
NGYPPEKVDVATGKAQGKGPVGFSAAMLPFLQNRDAQAVQRQRVADNFPGSDAY
YNYVLTLFGQGWDQHRFRFSTKGELLPDWGQECANSH

Endoglucanase (EC 3.2.1.4) (Carboxymethylcellulase) (CMCase) (Cellulase) (Endo-1,4-beta-glucanase) Escherichia coli (strain K12)

MNVLRSGIVTMLLLAAFSVQAACTWPAWEQFKKDYISQEGRVIDPSDARKITTSEGQ
SYGMFSALAANDRAAFDNILDWTQNNLAQGSLKERLPAWLWGKKENSKWEVLDS
NSASDGDVWMAWSLLEAGRLWKEQRYTDIGSALLKRIAREEVVTVPGLGSMLLPG
KVGFAEDNSWRFNPSYLPPTLAQYFTRFGAPWTTLRETNQRLLLETAPKGFSPDWVR
YEKDKGWQLKAEKTLISSYDAIRVYMWVGMMPDSDPQKARMLNRFKPMATFTEK
NGYPPEKVDVATGKAQGKGPVGFSAAMLPFLQNRDAQAVQRQRVADNFPGSDAY
YNYVLTLFGQGWDQHRFRFSTKGELLPDWGQECANSH

Endoglucanase A (EC 3.2.1.4) (Cellulase) (Endo-1,4-beta-glucanase) Fibrobacter succinogenes (Bacteroides succinogenes)

MNCRKYLLSGLAVFGLAATSAVAALSTDDYVEAAWMTTRFFGAQRSGQGPNWILD
GTSNPTSFTKDSYNGKDVSGGWFDCGDHVMYGQSQGYASYVLALAYAEFTEVSTT
FILVTTPTTRKPTTTPMKSGKPNKVRDLLEELRYEADFWVKAAIDGNNFVTVKGDG
NADHQKWVTAGAMSKLGSGEGGEPRCITGNANDGFTSGLAAAMLAVMARVDPDT
ANQAKYLKAAKTAYSYAKSHKGVTNSQGFYESSWWDGRWEDGPFLAELELYRTT
GENSYKTAAIDRYDNLKFSLGEGTHFMYSNVVPLSAVMAEAVFEETPHGMRKEAIG
VLDLIYEEKAKDKIFQNPNGMGSGKFPVRVPSGGAFLYALSDKFNNTNEHMEMIEK
NVSYLLGDNGSKKSYVVGFSKNGANAPSRPHHRGYYANEKRWRRSRRCSESSRKE
QALGRYDCWRLY

Endoglucanase A (EC 3.2.1.4) (Cellulase) (Endo-1,4-beta-glucanase) Fibrobacter succinogenes (Bacteroides succinogenes)

MNCRKYLLSGLAVFGLAATSAVAALSTDDYVEAAWMTTRFFGAQRSGQGPNWILD
GTSNPTSFTKDSYNGKDVSGGWFDCGDHVMYGQSQGYASYVLALAYAEFTEVSTT
FILVTTPTTRKPTTTPMKSGKPNKVRDLLEELRYEADFWVKAAIDGNNFVTVKGDG
NADHQKWVTAGAMSKLGSGEGGEPRCITGNANDGFTSGLAAAMLAVMARVDPDT
ANQAKYLKAAKTAYSYAKSHKGVTNSQGFYESSWWDGRWEDGPFLAELELYRTT
GENSYKTAAIDRYDNLKFSLGEGTHFMYSNVVPLSAVMAEAVFEETPHGMRKEAIG
VLDLIYEEKAKDKIFQNPNGMGSGKFPVRVPSGGAFLYALSDKFNNTNEHMEMIEK
NVSYLLGDNGSKKSYVVGFSKNGANAPSRPHHRGYYANEKRWRRSRRCSESSRKE
QALGRYDCWRLY

Endoglucanase 3 (EC 3.2.1.4) (Cellulase 3) (Endo-1,4-beta-glucanase 3) (EG3) Fibrobacter succinogenes (strain ATCC 19169/S85)

MQLKNFYPKMSVLGIATVMALTACGDENTQALFANNPVPGAENQVPVSSSDMSPTS
SDAVIDPTSSSAAVVDPSTLPAEGPITMPEGLGTLVDDFEDGDNLSKIGDYWYTYND
NDNGGASIITTPLNEEENIIPGRVNNGSNYALQVNYTLDRGDYEYDPYVGWGVQVA
PDEANGHFGGLTYWYKGGAHEVHIEITDVEDYDVHLAKFPASRTWKQAVVRFKDL
VQGGWGKEIPFDAKHIMAISFQAKGNKSKLVTDSLFIDNIYLQDSSEVEKDQPDMEIK
DPVIPVVEFTEAEITVTNPLQEKAMKYLNKGVNFTNWLENADGKFKSFELGESDVKI
LADNGFKSLRLPIDLDLYATNRDAFIAGTDTELKFDDDTLFLVLDSFVEWTAKYNMS
FVIDYHEYDNSYNTTSAKDPNYIKMMAETWKHVAAHYAESPREDLFFELLNEPDMS
DGKVTAATWTTAAQAMIDAIRTVDTKHTILFGDAQWYSITLLAKRTPFTDDNIIYVIH
TYEPFAFTHQGGSWTDYATIHDIPFPYDPAKWSTVSGDFGVNKSTKSYVKTNIKNYY
KTGSKEAILEQILKAKKWAATNNVPVIINEFGALNLRSTAESRLNYLTAMREICDTLQ
IPWTHWGYTGNFSVIENGKLIEGLDKALGVGSK

Endoglucanase type C (EC 3.2.1.4) (Cellulase) (Endo-1,4-beta-glucanase) (Endoglucanase I) (EG I) Fusarium oxysporum (Fusarium vascular wilt)

MKSLSLILSALAVQVAVAQTPDKAKEQHPKLETYRCTKASGCKKQTNYIVADAGIH
GIRQKNGAGCGDWGQKPNATACPDEASCAKNCILSGMDSNAYKNAGITTSGNKLRL
QQLINNQLVSPRVYLLEENKKKYEMLHLTGTEFSFDVEMEKLPCGMNGALYLSEMP
QDGGKSTSRNSKAGAYYGAGYCDAQCYVTPFINGVGNIKGQGVCCNELDIWEANS
RATHIAPHPCSKPGLYGCTGDECGSSGICDKAGCGWNHNRINVTDFYGRGKQYKVD
STRKFTVTSQFVANKQGDLIELHRHYIQDNKVIESAVVNISGPPKINFINDKYCAATG
ANEYMRLGGTKQMGDAMSRGMVLAMSVWWSEGDFMAWLDQGVAGPCDATEGD
PKNIVKVQPNPEVTFSNIRIGEIGSTSSVKAPAYPGPHRL

Cellulase (EC 3.2.1.4)Geobacillus sp. 70PC53

MKKAFLPVFIFLLVLTYFDKEGNAMERTPVEENGRLQVVGTALLNQHNKPFQLRGIS
THGLQWFGQFANKDAFQTLRDDWKANVVRLAMYTDPNANGYIAQPEWLKAKVKE
GVQAALDLGMYVIIDWHILNDNDPNLYKEQAKRFFAEMAREYGKYPNVIYEIANEP
NGNDVTWEEKIRPYADEVIRTIRSIDRDNLIIVGTGTWSQDVDDVASDPLPYKNIMYA
VHFYSGTHTQWLRDRVDAALQAGTPVFVSEWGTSDASGDGGPYLEEAEKWIEFLNE
RGISWVNWSLCDKNEASAALRPGADPHGGWGDDHLSDSGRFIKAKLIEAAQQSGQK
AKGAANPHQRNGNDSSDGGKTGHASGHPFFWTFILAAGVAFGFGGLALGKRLFKE

Endoglucanase (EC 3.2.1.4) (Alkaline cellulase) (Endo-1,4-beta-glucanase) Halalkalibacter akibai (strain ATCC 43226/DSM 21942/CIP 109018/JCM 9157/1139) (Bacillus akibai)

MMLRKKTKQLISSILILVLLLSLFPTALAAEGNTREDNFKHLLGNDNVKRPSEAGALQ
LQEVDGQMTLVDQHGEKIQLRGMSTHGLQWFPEILNDNAYKALANDWESNMIRLA
MYVGENGYASNPELIKSRVIKGIDLAIENDMYVIVDWHVHAPGDPRDPVYAGAEDF
FRDIAALYPNNPHIIYELANEPSSNNNGGAGIPNNEEGWNAVKEYADPIVEMLRDSG
NADDNIIIVGSPNWSQRPDLAADNPIDDHHTMYTVHFYTGSHAASTESYPPETPNSER
GNVMSNTRYALENGVAVFATEWGTSQANGDGGPYFDEADVWIEFLNENNISWAN
WSLTNKNEVSGAFTPFELGKSNATSLDPGPDQVWVPEELSLSGEYVRARIKGVNYEP
IDRTKYTKVLWDFNDGTKQGFGVNGDSPVEDVVIENEAGALKLSGLDASNDVSEGN
YWANARLSADGWGKSVDILGAEKLTMDVIVDEPTTVSIAAIPQGPSANWVNPNRAI
KVEPTNFVPLEDKFKAELTITSADSPSLEAIAMHAENNNINNIILFVGTEGADVIYLDN
IKVIGTEVEIPVVHDPKGEAVLPSVFEDGTRQGWDWAGESGVKTALTIEEANGSNAL
SWEFGYPEVKPSDNWATAPRLDFWKSDLVRGENDYVTFDFYLDPVRATEGAMNIN
LVFQPPTNGYWVQAPKTYTINFDELEEPNQVNGLYHYEVKINVRDITNIQDDTLLRN
MMIIFADVESDFAGRVFVDNVRFEGAATTEPVEPEPVDPGEETPPVDEKEAKTEQKE
AEKEEKEE

Endoglucanase (EC 3.2.1.4) (Alkaline cellulase) (Endo-1,4-beta-glucanase) Halalkalibacter akibai (strain ATCC 43226/DSM 21942/CIP 109018/JCM 9157/1139) (Bacillus akibai)

MMLRKKTKQLISSILILVLLLSLFPTALAAEGNTREDNFKHLLGNDNVKRPSEAGALQ
LQEVDGQMTLVDQHGEKIQLRGMSTHGLQWFPEILNDNAYKALANDWESNMIRLA
MYVGENGYASNPELIKSRVIKGIDLAIENDMYVIVDWHVHAPGDPRDPVYAGAEDF
FRDIAALYPNNPHIIYELANEPSSNNNGGAGIPNNEEGWNAVKEYADPIVEMLRDSG
NADDNIIIVGSPNWSQRPDLAADNPIDDHHTMYTVHFYTGSHAASTESYPPETPNSER
GNVMSNTRYALENGVAVFATEWGTSQANGDGGPYFDEADVWIEFLNENNISWAN
WSLTNKNEVSGAFTPFELGKSNATSLDPGPDQVWVPEELSLSGEYVRARIKGVNYEP
IDRTKYTKVLWDFNDGTKQGFGVNGDSPVEDVVIENEAGALKLSGLDASNDVSEGN
YWANARLSADGWGKSVDILGAEKLTMDVIVDEPTTVSIAAIPQGPSANWVNPNRAI
KVEPTNFVPLEDKFKAELTITSADSPSLEAIAMHAENNNINNIILFVGTEGADVIYLDN
IKVIGTEVEIPVVHDPKGEAVLPSVFEDGTRQGWDWAGESGVKTALTIEEANGSNAL
SWEFGYPEVKPSDNWATAPRLDFWKSDLVRGENDYVTFDFYLDPVRATEGAMNIN
LVFQPPTNGYWVQAPKTYTINFDELEEPNQVNGLYHYEVKINVRDITNIQDDTLLRN
MMIIFADVESDFAGRVFVDNVRFEGAATTEPVEPEPVDPGEETPPVDEKEAKTEQKE
AEKEEKEE

Endoglucanase EG-1 (EC 3.2.1.4) (Cellulase) (Endo-1,4-beta-glucanase) Hypocrea jecorina (Trichoderma reesei)

MAPSVTLPLTTAILAIARLVAAQQPGTSTPEVHPKLTTYKCTKSGGCVAQDTSVVLD
WNYRWMHDANYNSCTVNGGVNTTLCPDEATCGKNCFIEGVDYAASGVTTSGSSLT
MNQYMPSSSGGYSSVSPRLYLLDSDGEYVMLKLNGQELSFDVDLSALPCGENGSLY
LSQMDENGGANQYNTAGANYGSGYCDAQCPVQTWRNGTLNTSHQGFCCNEMDIL
EGNSRANALTPHSCTATACDSAGCGFNPYGSGYKSYYGPGDTVDTSKTFTIITQFNT
DNGSPSGNLVSITRKYQQNGVDIPSAQPGGDTISSCPSASAYGGLATMGKALSSGMV
LVFSIWNDNSQYMNWLDSGNAGPCSSTEGNPSNILANNPNTHVVFSNIRWGDIGSTT
NSTAPPPPPASSTTFSTTRRSSTTSSSPSCTQTHWGQCGGIGYSGCKTCTSGTTCQYSN
DYYSQCL

Glucanase (EC 3.2.1.→Klebsiella variicola

MPLRALVAVIVTTAVMLVPRAWADTAWERYKARFMMPDGRIIDTANGNVSHTEGQ
GFAMLLAVANNDRPAFDKLWQWTDSTLRDKSNGLFYWRYNPVAPDPIADKNNASD
GDTLIAWALLRAQKQWQDKRYAIASDAITASLLKYTVVTFAGRQVMLPGVKGFNL
NDHLNLNPSYFIFPAWRAFAERTHLTAWRTLQTDGQALLGQMGWGKSHLPSDWVA
LRADGKMLPAKEWPPRMSFDAIRIPLYLSWADPQSALLAPWKAWMQSYPRLQTPA
WINVSTNEVAPWYMAGGLLAVRDLTLGEPQEAPQIDDKDDYYSASLKQLVWLAKQ
DQR

Cellulase (EC 3.2.1.4)Komagataeibacter sucrofermentans

MAVAGSFPMLSSGAEADDAIGINPQIAQQWAIFRDKYFHPNGRIIDTGNSGESHSEGQ
GYGMLFSAAAGDQAAFEVIWVWARTNLQHKDDALFSWRYLDGHKPPVADKNNAT
DGDLLIALALAWAGKRWKRADYIQDAMNIYGDVLKLMTKSVGPYTVLLPGAVGFL
TKDTVTLNLSYYVMPSLMQAFALTGDAKWTKVMGDGLQIIAKGRFGEWKLPPDWL
SINLHTNAFSIAKGWPPRFSYDAIRVPLYLSWAHMLTPELLADFSRFWNHYGASALP
GWVDLTNGARSPYNAPPGYLAVASCTGLASAGELPTLDHAPDYYSAALTMLAYIAR
NQGDGM

Endoglucanase (EC 3.2.1.4) Martelella endophytica

MPRMLAASAAIIATTLAPLSAQAAGCEMTLHGINLSGAEFGQPGDPYGQGYIYPSEST
IKAFADDGFNAVRLPFLWERLQPTLNGTLDATELSRIKDTVETLRDNGMVVILDVHN
YARYHGEMIGTPNVPVAAFADFWKRLSAVFANDDDVIFGLMNEPHDISAPAWLAA
ANAAIDAIRTIGAGNLVLVPGTAWTGAHSWSQTFYGPSNASVMAQVVDSSNNFAYE
VHQYTDDDFSGKNADCSKIDDAVSALNDFTSWLNANDVQGFLGEFGTTEQIQCLRG
LKQMVDVVQQNPRAWLGWAYWAGGDWWPKDSPMIIHSNPRDGGTQQLRTLQPVL
GSNTTRASCNTKS

Endoglucanase (EC 3.2.1.4) Martelella endophytica

MPRMLAASAAIIATTLAPLSAQAAGCEMTLHGINLSGAEFGQPGDPYGQGYIYPSEST
IKAFADDGFNAVRLPFLWERLQPTLNGTLDATELSRIKDTVETLRDNGMVVILDVHN
YARYHGEMIGTPNVPVAAFADFWKRLSAVFANDDDVIFGLMNEPHDISAPAWLAA
ANAAIDAIRTIGAGNLVLVPGTAWTGAHSWSQTFYGPSNASVMAQVVDSSNNFAYE
VHQYTDDDFSGKNADCSKIDDAVSALNDFTSWLNANDVQGFLGEFGTTEQIQCLRG
LKQMVDVVQQNPRAWLGWAYWAGGDWWPKDSPMIIHSNPRDGGTQQLRTLQPVL
GSNTTRASCNTKS

Endoglucanase H (EC 3.2.1.4) Meiothermus taiwanensis

MRFLAVSGLLLVLGPVGCQSTQLQTPAPDTGGIVELNRQLGRGVNLGNALEAPWEG
AWGVRLEEGFFELIREAGFKTIRLPVSWTHHAGRAAPYTIDPAFFSRVDWAVTQATR
RGLNIVVNVHHYDELNANPQAEEARYLSIWRQIAERYRNQPGSVYFELLNEPHGREN
DNPQLWNDLLAKALRVVRESNPSRAVIVGPVGWNSLWRLSELRLPDDPNLIVTFHY
YDPLEFTHQGAEWLNPVPPTGVVWRENQGAFAAGWQNWSWGSRVGFVGEALEIT
YQEGWAGFYLHSDAGVEGYDRLAFRTSAPVSLQVSCRRDAPAKAVTTSGGVETVV
NLSECGNPSRLTDLILQNNSPNARAAFRLERLELRGPGSPLALLTHQQNAIAQAMEFA
QRWAEQNRRPIFVGEFGAYEKGDLDSRVRWTGAVRSELEKRNFSWAYWEFAAGFG
IYDRTTRQWRTPLLKALVPEQP

Endoglucanase MaCel5A (EC 3.2.1.4) (Endo-beta-1,4-glucanase) Microbulbifer sp. (strain ALW1)

MKRILFTAGGCLFYLLLAVKAYAYDCSATPAYQDGVNYQSGDLVSNTGAAYRCNV
AGWCSTGGAYAPGTGWAWTEAWDELGSCDGGAGSSGSSSSSSSSSSSSSGSSSSSSG
SGSSSGGTSCDGIEAWQVASIYTEGNVVQQNGERYIANWWNQGQSPEDNSGAYEV
WSAAGSCSGAGSSSSSSGGTSSSGSSSSGVSSSGGSSGGDSPIARHGKLHVCGNGLCN
ADNVPVQLRGMSTHGLQWYGWGNCITGNSLDTLAEDWNADILRVSLYVQEGGYET
DPAGYTAQVSHIIDEVTARGMYVLVDWHQLDPGDPNANLDNARQFFTDIAQAHGD
KTNIIYDVANEPNNVSWDAIQRYAMEVIPVIRQYAPDAVVLVGTHGWASLGISDGGS
AQDIFNNPVTIDNIMYTFHFYAASHGQVYRDELRSALERGMPVFVTEWGSQTYTGD
DGNDFVSTQAYLDLLDQYQISWTNWNYSDDFRTGAVWNTGTCSADSWGVGNLKE
AGAWVRDKIRNR

Probable endoglucanase (EC 3.2.1.4) (Cellulase) (Endo-1,4-beta-glucanase) Novacetimonas hansenii (Komagataeibacter hansenii)

MSVMAAMGGAQVLSSTGAFADPAPDAVAQQWAIFRAKYLRPSGRVVDTGNGGES
HSEGQGYGMLFAASAGDLASFQSMWMWARTNLQHTNDKLFSWRFLKGHQPPVPD
KNNATDGDLLIALALGRAGKRFQRPDYIQDAMAIYGDVLNLMTMKAGPYVVLMPG
AVGFTKKDSVILNLSYYVMPSLLQAFDLTADPRWRQVMEDGIRLVSAGRFGQWRLP
PDWLAVNRATGALSIASGWPPRFSYDAIRVPLYFYWAHMLAPNVLADFTRFWNNFG
ANALPGWVDLTTGARSPYNAPPGYLAVAECTGLDSAGELPTLDHAPDYYSAALTLL
VYIARAEETIK

Probable endoglucanase (EC 3.2.1.4) (Cellulase) (Endo-1,4-beta-glucanase) Novacetimonas hansenii (Komagataeibacter hansenii)

MSVMAAMGGAQVLSSTGAFADPAPDAVAQQWAIFRAKYLRPSGRVVDTGNGGES
HSEGQGYGMLFAASAGDLASFQSMWMWARTNLQHTNDKLFSWRFLKGHQPPVPD
KNNATDGDLLIALALGRAGKRFQRPDYIQDAMAIYGDVLNLMTMKAGPYVVLMPG
AVGFTKKDSVILNLSYYVMPSLLQAFDLTADPRWRQVMEDGIRLVSAGRFGQWRLP
PDWLAVNRATGALSIASGWPPRFSYDAIRVPLYFYWAHMLAPNVLADFTRFWNNFG
ANALPGWVDLTTGARSPYNAPPGYLAVAECTGLDSAGELPTLDHAPDYYSAALTLL
VYIARAEETIK

cellulase (EC 3.2.1.4) Novosphingobium sp

MMNLNRRTFSIGMLLAMTAACNTGTQSHAEGRPFTGSLAGNWWPLYRQRFLAPEG
RIVDNGNGGISHSEGQGYGMVLAALAGDRASFAKMADWADRTLLRKDMALYSWR
YDPKLANPVSDPNNATDGDVLIAWALSIAAARWGNSAWRQRSAAVRADIRAKCVA
TRYGREILVPGIAGFVETNGITINPSYFIWSAMEAFAKLDGEAAWRGIINDADALLRL
SRFGAHGVPTDWVLVTGRDQVAPAPDKPPRFGFDAIRVPLYATMAGRTGLTDNIAA
YWRGCLAQGRPIPAWVDVMTGEEANYAVSEGGAAIVSRLLNTAAPSQLSNDYFAAS
LQMLAQARP

Protein names Organism Sequence

Endoglucanase A (EC 3.2.1.4) (Cellulase A) (Endo-1,4-beta-D-glucanase A) Paenibacillus barcinonensis

MTKTFKKFSIAGLALLFMATAAFAGWSTKASAADMRSLTAAQITAEMGAGWNLGN
QLEATVNGTPNETSWGNPTITPELIKKVKAAGFKTIRIPVSYLNYIGSAPNYTVNASW
LNRIQQVVDYAYNEGLYVVINMHGDGFHSIPGSWLHVNSSNQNVIRDKYQKVWQQ
VATRFSAYNERLIFESMNEVFDGNYNNPNTSYYGNLNAYNQIFVDTVRKTGGNNNA
RWLLVPGWNTNIDYTVGNYGFVVPTDNFRSSAIPSSQKRIMISAHYYSPWDFAGEEN
GNITQWGATATNPAKRSTWGQEDYLDSQFKSMYDKFVTQGYPVVMGEFGSIDKSS
YDSSNNNYRAVYAKAVTATAKKYKLVPVYWDNGFNGQHGFALFNRFNNTVTQQN
IINAIMQGMQ

Endoglucanase A (EC 3.2.1.4) (Cellulase A) (Endo-1,4-beta-D-glucanase A) Paenibacillus barcinonensis

MTKTFKKFSIAGLALLFMATAAFAGWSTKASAADMRSLTAAQITAEMGAGWNLGN
QLEATVNGTPNETSWGNPTITPELIKKVKAAGFKTIRIPVSYLNYIGSAPNYTVNASW
LNRIQQVVDYAYNEGLYVVINMHGDGFHSIPGSWLHVNSSNQNVIRDKYQKVWQQ
VATRFSAYNERLIFESMNEVFDGNYNNPNTSYYGNLNAYNQIFVDTVRKTGGNNNA
RWLLVPGWNTNIDYTVGNYGFVVPTDNFRSSAIPSSQKRIMISAHYYSPWDFAGEEN
GNITQWGATATNPAKRSTWGQEDYLDSQFKSMYDKFVTQGYPVVMGEFGSIDKSS
YDSSNNNYRAVYAKAVTATAKKYKLVPVYWDNGFNGQHGFALFNRFNNTVTQQN
IINAIMQGMQ

cellulase (EC 3.2.1.4) Paenibacillus pabuli

MFKKWKKFGISSLALVLVAAVAFTGWSAKASAADASQIVSEMGAGWNLGNQLEAA
VNGTPNETAWGNPTVTPELIKKVKAAGFKSIRIPVSYLNNIGSAPNYTINAAWLNRIQ
QVVDYAYNEGLYVIINIHGDGYNSVQGGWLLVNGGNQTAIKEKYKKVWQQIATKF
SNYNDRLIFESMNEVFDGNYGNPNSAYYTNLNAYNQIFVDTVRQTGGNNNARWLL
VPGWNTNIDYTVGNYGFTLPTDNYRSSAIPSSQKRIMISAHYYSPWDFAGEENGNITQ
WGATSTNPAKKSTWGQEDYLESQFKSMYDKFVTQGYPVVIGEFGSIDKTSYDSSNN
VYRAAYAKAVTAKAKKYKMVPVYWDNGHNGQHGFALFNRSNNTVTQQNIINAIM
QGMQ

Endoglucanase 5 (EC 3.2.1.4) (Cellulase V) (Endo-1,4-beta-glucanase V) (Endoglucanase V) Pectobacterium carotovorum subsp. carotovorum (Erwinia carotovora subsp. carotovora)

MWMRRNQIVRKLTLGVVTTVLGMSLSFSALSATPVETHGQLSIENGRLVDEQGKRV
QLRGISSHGLQWFGDYVNKDSMKWLRDDWGINVFRVAMYTAADGYISNPSLANKV
KEAVAAAQSLGVYIIIDWHILSDNDPNIYKAQAKTFFAEMAGLYGSSPNVIYEIANEP
NGGVTWNGQIRPYALEVTDTIRSKDPDNLIIVGTGTWSQDIHDAADNQLPDPNTMY
ALHFYAGTHGQFLRDRIDYAQSRGAAIFVSEWGTSDASGNGGPFLPESQTWIDFLNN
RGVSWVNWSLTDKSEASAALAPGASKSGGWTEQNLSTSGKFVREQIRAGANLGGG
DTPTTPTEPTNPGNGTTGDVVLQYRNVDNNPSDDAIRMAVNIKNTGSTPIKLSDLQV
RYYFHDDGKPGANLFVDWANVGPNNIVTSTGTPAASTDKANRYVLVTFSSGAGSLQ
PGAETGEVQVRIHAGDWSNVNETNDYSYGANVTSYANWDKITVHDKGTLVWGVEP

Endoglucanase 5 (EC 3.2.1.4) (Cellulase V) (Endo-1,4-beta-glucanase V) (Endoglucanase V) Pectobacterium carotovorum subsp. carotovorum (Erwinia carotovora subsp. carotovora)

MWMRRNQIVRKLTLGVVTTVLGMSLSFSALSATPVETHGQLSIENGRLVDEQGKRV
QLRGISSHGLQWFGDYVNKDSMKWLRDDWGINVFRVAMYTAADGYISNPSLANKV
KEAVAAAQSLGVYIIIDWHILSDNDPNIYKAQAKTFFAEMAGLYGSSPNVIYEIANEP
NGGVTWNGQIRPYALEVTDTIRSKDPDNLIIVGTGTWSQDIHDAADNQLPDPNTMY
ALHFYAGTHGQFLRDRIDYAQSRGAAIFVSEWGTSDASGNGGPFLPESQTWIDFLNN
RGVSWVNWSLTDKSEASAALAPGASKSGGWTEQNLSTSGKFVREQIRAGANLGGG
DTPTTPTEPTNPGNGTTGDVVLQYRNVDNNPSDDAIRMAVNIKNTGSTPIKLSDLQV
RYYFHDDGKPGANLFVDWANVGPNNIVTSTGTPAASTDKANRYVLVTESSGAGSLQ
PGAETGEVQVRIHAGDWSNVNETNDYSYGANVTSYANWDKITVHDKGTLVWGVEP

Endoglucanase 5 (EC 3.2.1.4) (Cellulase V) (Endo-1,4-beta-glucanase V) Pectobacterium parmentieri

MQTVNTQPHRIFRVLLPAVFSSLLLSSLTVSAASSSNDADKLYFGNNKYYLFNNVWG
KDEIKGWQQTIFYNSPISMGWNWHWPSSTHSVKAYPSLVSGWHWTAGYTENSGLPI
QLSSNKSITSNVTYSIKATGTYNAAYDIWFHTTDKANWDSSPTDELMIWLNDTNAGP
AGDYIETVFLGDSSWNVFKGWINADNGGGWNVFSFVHTSGTNSASLNIRHFTDYLV
QTKQWMSDEKYISSVEFGTEIFGGDGQIDITEWRVDVK

Manganese dependent endoglucanase Eg5A (EC 3.2.1.4) (Carboxymethyl-cellulase 5A) (CMCase 5A) (Cellulase 5A) (Endo-1,4-beta-glucanase Eg5A) Phanerodontia chrysosporium (White-rot fungus) (Sporotrichum pruinosum)

MLKYASIALALATLGVAQQQQWGQCGGIGWTGATTCVAGSVCSVLNPYYSQCIPG
AATVTSSSAPSTPTPPAGALPRLGGVNTAGYDFSVATDGSFTGTGVSPPVSQFSHFSS
QGANLYRIPFAWQLMTPTLGGTISQSFLSRYDQTVQAALNSGPNVFVIIDLHNYARW
NGGIIAQGGPTDAQFQSIWTQLAQKYGSNQRVIFGIMNEPHDIPSISTWVNSVQGAVN
AIRAAGATNYLLLPGSSWSSAQAFPTEAGPLLVKVTDPLGGTSKLIFDVHKYLDSDN
SGTHPDCTTDNVQVLQTLVQFLQANGNRQAILSETGGGNTSSCESLLANELAYVKSA
YPTLAGFSVWAAGAFDTTYVLTVTPNADGSDQPLWVDAVKPNLP

Cellulase (EC 3.2.1.4) Phocaeicola salanitronis (strain DSM 18170/JCM 13657/BL78) (Bacteroides salanitronis)

MRNCLTLFFLLLTGIALFASCSDDEGGGYTGETQISATEMTFPKSGDTLTLSVLSSVEP
QVTSNQTWCNVSFASVTERGTYTYNVTVDANSETDDRVATLSVVAGSFTASVQVTQ
TAGDGLIVAQTAYDMPAAGGALSITLTTNGDYAYTINDSWIAEAEVTDARAMTEYT
LHFTVSANHGTERTGTITFTLGDLTETVTVTQAAGQAAAVSGETPLEIAVSLGLGWN
LGNQLDAHNNGVADETSWGNAAATQALFDALANAGFTSVRIPVTWLGHVGEAPDY
TIDETYLNRVAEVVGYAESAGLNAIINIHHDGANSQYWLDIKDAATDETVNSAVKA
QLAAMWTQIANRFADKGNFLVFEAMNEIHDGSWGWGDNRTDGGRQYAVLNEWN
QVFVDAVRATGGNNQTRYLGVPGYVTNIDLTVENFVLPQDVVDNRLMVAVHFYDP
IDYTENADNIYSQWGHTADPSLKADWGDEDNVTGQFAKMKETFIDQGIPAYIGEMG
CVHRADDLSESFRLYYLEYVCKAAKDYGMPPFYWDAGGDGTGTQSWALFNHATG
EMLNNAQEVIDVMKRGIFTEDASYTLQSVYDSAPQ

Cellulase (EC 3.2.1.4)Pseudoalteromonas haloplanktis (Alteromonas haloplanktis)

MNNSSNNHKRKDFKVASLSLALLLGCSTMANAAVEKLTVSGNQILAGGENTSFAGP
SLFWSNTGWGAEKFYTAETVAKAKTEFNATLIRAAIGHGTSTGGSLNFDWEGNMSR
LDTVVNAAIAEDMYVIIDFHSHEAHTDQATAVRFFEDVATKYGQYDNVIYEIYNEPL
QISWVNDIKPYAETVIDKIRAIDPDNLIVVGTPTWSQDVDVASQNPIDRANIAYTLHF
YAGTHGQSYRNKAQTALDNGIALFATEWGTVNADGNGGVNINETDAWMAFFKTN
NISHANWALNDKNEGASLFTPGGSWNSLTSSGSKVKEIIQGWGGGSSNVDLDSDGD
GVSDSLDQCNNTPAGTTVDSIGCAVTDSDADGISDNVDQCPNTPVGETVNNVGCVV
EVVEPQSDADNDGVNDDIDQCPDTPAGTSVDTNGCSVVSSTDCNGINAYPNWVNKD
YSGGPFTHNNTDDKMQYQGNAYSANWYTNSLPGSDASWTLLYTCN

Cellulase (EC 3.2.1.4)Pseudoalteromonas haloplanktis (Alteromonas haloplanktis)

MKVFFKITTLLLILISYQSLAAFNNNPSSVGAYSSGTYRNLAQEMGKTNIQQKVNSTF
DNMFGYNNTQQLYYPYTENGVYKAHYIKAINPDEGDDIRTEGQSWGMTAAVMLNK
QEEFDNLWRFAKAYQKNPDNHPDAKKQGVYAWKLKLNQNGFVYKVDEGPAPDGE
EYFAFALLNASARWGNSGEFNYYNDAITMLNTIKNKLMENQIIRFSPYIDNLTDPSYH
IPAFYDYFANNVTNQADKNYWRQVATKSRTLLKNHFTKVSGSPHWNLPTFLSRLDG
SPVIGYIFNGQANPGQWYEFDAWRVIMNVGLDAHLMGAQAWHKSAVNKALGFLS
YAKTNNSKNCYEQVYSYGGAQNRGCAGEGQKAANAVALLASTNAGQANEFFNEF
WSLSQPTGDYRYYNGSLYMLAMLHVSGNFKFYNNTFN

Glucanase (EC 3.2.1.→Pseudomonas putida (strain ATCC 47054/DSM 6125/CFBP 8728/NCIMB 11950/KT2440)

MRQMLVGLVALGLPVFANAGSVCPWPAWERFKAELVSVDGRVIDPSDERLITTSEG
QSYALFFALVGNDRQTFAQLLRWTSNNLAEGDLARHLPAWLWGRDGQQQWQVLD
ANNASDADLWIAYSLLEAGRLWDQPAYTQLGQHLLWRIAAQTVRKLPGLGVMLLP
GDYGFEDAQGTRLNPSYLPLQLLDRFSDVDPLWGELAANTRRLWLASSPKGFAPDW
LLWTPAGKPAADTKHGNAGDYDAIRVYLWVGMLAEGAAQRRELVAHYAPMAALT
QRQGLPPEHLDARSGEARGHGPAGFSAALLPLLAASPEHVAGLAAQRQRLREQPVE
AKAYYSQVLALFGQGFDEARYRFDPHGRLLPAWSAPCSE

Endoglucanase (EC 3.2.1.4) (Cellulase) (Endo-1,4-beta-glucanase) Ralstonia solanacearum (Pseudomonas solanacearum)

MRRCMPLVAASVAALMLAGCGGGDGDPSLSTASVSATDTTTLKPAATSTTSSVWLT
LAKDSAAFTVSGTRTVRYGAGSAWVEKSVSGSGRCTSTFFGKDPAAGVAKVCQLLQ
GTGTLLWRGVSLAGAEFGEGSLPGTYGSNYIYPSADSVTYYKNKGMNLVRLPFRWE
RLQPTLNQVFDANELSRLTGFVNAVTATGQTVLLDPHNYARYYGNVIGSSAVPNSA
YADFWRRLATQFKSNPRVILGLMNEPNSMPTEQWLSGANAELAAIRSANASNVVFV
PGNAWTGAHSWNQNWYGTPNGTVMKGINDPGHNLVFEVHQYLDGDSSGQSANCV
SATIGAQRLQDFTTWLRSNGYRGFLGEFGAASNDTCNQAVSNMLTFVKNNADVWT
GWAWWAGGPWWGGYMYSIEPSNGVDKPQMSVLAPYLK

Endoglucanase (EC 3.2.1.4) (Cellulase) (Endo-1,4-beta-glucanase) Ralstonia solanacearum (Pseudomonas solanacearum)

MRRCMPLVAASVAALMLAGCGGGDGDPSLSTASVSATDTTTLKPAATSTTSSVWLT
LAKDSAAFTVSGTRTVRYGAGSAWVEKSVSGSGRCTSTFFGKDPAAGVAKVCQLLQ
GTGTLLWRGVSLAGAEFGEGSLPGTYGSNYIYPSADSVTYYKNKGMNLVRLPFRWE
RLQPTLNQVFDANELSRLTGFVNAVTATGQTVLLDPHNYARYYGNVIGSSAVPNSA
YADFWRRLATQFKSNPRVILGLMNEPNSMPTEQWLSGANAELAAIRSANASNVVFV
PGNAWTGAHSWNQNWYGTPNGTVMKGINDPGHNLVFEVHQYLDGDSSGQSANCV
SATIGAQRLQDFTTWLRSNGYRGFLGEFGAASNDTCNQAVSNMLTFVKNNADVWT
GWAWWAGGPWWGGYMYSIEPSNGVDKPQMSVLAPYLK

Cellulase (EC 3.2.1.4)Rhodothermus marinus (Rhodothermus obamensis)

MNVMRAVLVLSLLLLFGCDWLFPDGDNGKEPEPEPEPTVELCGRWDARDVAGGRY
RVINNVWGAETAQCIEVGLETGNFTITRADHDNGNNVAAYPAIYFGCHWAPARAIR
DCAARAGAVRRAHELDVTPITTGRWNAAYDIWFSPVTNSGNGYSGGAELMIWLNW
NGGVMPGGSRVATVELAGATWEVWYADWDWNYIAYRRTTPTTSVSELDLKAFIDD
AVARGYIRPEWYLHAVETGFELWEGGAGLRTADFSVTVQ

Glucanase (EC 3.2.1.→Ruminiclostridium cellulolyticum

MKSKLIKLSAIIISGVMLTTSFVNTGTAFAAGTHDYSTALKDSIIFFDANKCGPQAGEN
NVFDWRGACHTTDGSDVGVDLTGGYHDAGDHVKFGLPQGYSAAILGWSLYEFKES
FDATGNTTKMLQQLKYFTDYFLKSHPNSTTFYYQVGEGNADHTYWGAPEEQTGQR
PSLYKADPSSPASDILSETSAALTLMYLNYKNIDSAYATKCLNAAKELYAMGKANQ
GVGNGQSFYQATSFGDDLAWAATWLYTATNDSTYITDAEQFITLGNTMNENKMQD
KWTMCWDDMYVPAALRLAQITGKQIYKDAIEINFNYWKTQVTTTPGGLKWLSNWG
VLRYAAAESMVMLVYCKQNPDQSLLDLAKKQVDYILGDNPANMSYIIGYGSNWCI
HPHHRAANGYTYANGDNAKPAKHLLTGALVGGPDQNDKFLDDANQYQYTEVALD
YNAGLVGVLAGAIKFFGGTIVNPPVKKGDLNNDTFIDAIDLALCKNYILTQNGNIDK
NNADMNGDGSIDAIDFSLLKKAILG

Endoglucanase A (EC 3.2.1.4) (Cellulase A) (EGCCA) (Endo-1,4-beta-glucanase A) Ruminiclostridium cellulolyticum (strain ATCC 35319/DSM 5812/JCM 6584/H10) (Clostridium cellulolyticum)

MKKTTAFLLCFLMIFTALLPMQNANAYDASLIPNLQIPQKNIPNNDGMNFVKGLRLG
WNLGNTFDAFNGTNITNELDYETSWSGIKTTKQMIDAIKQKGFNTVRIPVSWHPHVS
GSDYKISDVWMNRVQEVVNYCIDNKMYVILNTHHDVDKVKGYFPSSQYMASSKKY
ITSVWAQIAARFANYDEHLIFEGMNEPRLVGHANEWWPELTNSDVVDSINCINQLNQ
DFVNTVRATGGKNASRYLMCPGYVASPDGATNDYFRMPNDISGNNNKIIVSVHAYC
PWNFAGLAMADGGTNAWNINDSKDQSEVTWFMDNIYNKYTSRGIPVIIGECGAVDK
NNLKTRVEYMSYYVAQAKARGILCILWDNNNFSGTGELFGFFDRRSCQFKFPEIIDG
MVKYAFEAKTDPDPVIVYGDYNNDGNVDALDFAGLKKYIMAADHAYVKNLDVNL
DNEVNAFDLAILKKYLLGMVSKLPSN

Endoglucanase C (EC 3.2.1.4) (Cellulase C) (EGCCC) (Endo-1,4-beta-glucanase C) Ruminiclostridium cellulolyticum (strain ATCC 35319/DSM 5812/JCM 6584/H10) (Clostridium cellulolyticum)

MIKGSSLKRFKSLVMAAIFSVSIISTAIASSAADQIPFPYDAKYPNGAYSCLADSQSIGN
NLVRSEWEQWKSAHITSNGARGYKRVQRDASTNYDTVSEGLGYGLLLSVYFGEQQ
LFDDLYRYVKVFLNSNGLMSWRIDSSGNIMGKDSIGAATDADEDIAVSLVFAHKKW
GTSGGFNYQTEAKNYINNIYNKMVEPGTYVIKAGDTWGGSNVTNPSYFAPAWYRIF
ADFTGNSGWINVANKCYEIADKARNSNTGLVPDWCTANGTPASGQGFDFYYDAIRY
QWRAAIDYSWYGTAKAKTHCDAISNFFKNIGYANIKDGYTISGSQISSNHTATFVSC
AAAAAMTGTDTTYAKNIYNECVKVKDSGNYTYFGNTLRMMVLLYTTGNFPNLYTY
NSQPKPDLKGDVNNDGAIDALDIAALKKAILTQTTSNISLTNADMNNDGNIDAIDFA
QLKVKLLN

Endoglucanase F (EC 3.2.1.4) (Cellulase F) (EGCCF) (Endo-1,4-beta-glucanase F) Ruminiclostridium cellulolyticum (strain ATCC 35319/DSM 5812/JCM 6584/H10) (Clostridium cellulolyticum)

MSKNFKRVGAVAVAAAMSLSIMATTSINAASSPANKVYQDRFESMYSKIKDPANGY
FSEQGIPYHSIETLMVEAPDYGHVTTSEAMSYYMWLEAMHGRFSGDFTGFDKSWSV
TEQYLIPTEKDQPNTSMSRYDANKPATYAPEFQDPSKYPSPLDTSQPVGRDPINSQLT
SAYGTSMLYGMHWILDVDNWYGFGARADGTSKPSYINTFQRGEQESTWETIPQPCW
DEHKFGGQYGFLDLFTKDTGTPAKQFKYTNAPDADARAVQATYWADQWAKEQGK
SVSTSVGKATKMGDYLRYSFFDKYFRKIGQPSQAGTGYDAAHYLLSWYYAWGGGI
DSTWSWIIGSSHNHFGYQNPFAAWVLSTDANFKPKSSNGASDWAKSLDRQLEFYQW
LQSAEGAIAGGATNSWNGRYEAVPSGTSTFYGMGYVENPVYADPGSNTWFGMQV
WSMQRVAELYYKTGDARAKKLLDKWAKWINGEIKFNADGTFQIPSTIDWEGQPDT
WNPTQGYTGNANLHVKVVNYGTDLGCASSLANTLTYYAAKSGDETSRQNAQKLLD
AMWNNYSDSKGISTVEQRGDYHRFLDQEVFVPAGWTGKMPNGDVIKSGVKFIDIRS
KYKQDPEWQTMVAALQAGQVPTQRLHRFWAQSEFAVANGVYAILFPDQGPEKLLG
DVNGDETVDAIDLAILKKYLLNSSTTINTANADMNSDNAIDAIDYALLKKALLSIQ

Endoglucanase G (EC 3.2.1.4) (Cellulase G) (EGCCG) (Endo-1,4-beta-glucanase G) Ruminiclostridium cellulolyticum (strain ATCC 35319/DSM 5812/JCM 6584/H10) (Clostridium cellulolyticum)

MLKTKRKLTKAIGVALSISILSSLVSFIPQTNTYAAGTYNYGEALQKSIMFYEFQRSG
DLPADKRDNWRDDSGMKDGSDVGVDLTGGWYDAGDHVKFNLPMSYTSAMLAWS
LYEDKDAYDKSGQTKYIMDGIKWANDYFIKCNPTPGVYYYQVGDGGKDHSWWGP
AEVMQMERPSFKVDASKPGSAVCASTAASLASAAVVFKSSDPTYAEKCISHAKNLF
DMADKAKSDAGYTAASGYYSSSSFYDDLSWAAVWLYLATNDSTYLDKAESYVPN
WGKEQQTDIIAYKWGQCWDDVHYGAELLLAKLINKQLYKDSIEMNLDFWTTGVN
GTRVSYTPKGLAWLFQWGSLRHATTQAFLAGVYAEWEGCTPSKVSVYKDFLKSQID
YALGSTGRSFVVGYGVNPPQHPHHRTAHGSWTDQMTSPTYHRHTIYGALVGGPDN
ADGYTDEINNYVNNEIACDYNAGFTGALAKMYKHSGGDPIPNFKAIEKITNDEVIIKA
GLNSTGPNYTEIKAVVYNQTGWPARVTDKISFKYFMDLSEIVAAGIDPLSLVTSSNYS
EGKNTKVSGVLPWDVSNNVYYVNVDLTGENIYPGGQSACRREVQFRIAAPQGTTY
WNPKNDFSYDGLPTTSTVNTVTNIPVYDNGVKVFGNEPAGGSENPDPEILYGDVNSD
KNVDALDFAALKKYLLGGTSSIDVKAADTYKDGNIDAIDMATLKKYLLGTITQLPQ
G

Endoglucanase A (EC 3.2.1.4) (Cellulase A) (EGCCA) (Endo-1,4-beta-glucanase A) Ruminiclostridium cellulolyticum (strain ATCC 35319/DSM 5812/JCM 6584/H10) (Clostridium cellulolyticum)

MKKTTAFLLCFLMIFTALLPMQNANAYDASLIPNLQIPQKNIPNNDGMNFVKGLRLG
WNLGNTFDAFNGTNITNELDYETSWSGIKTTKQMIDAIKQKGFNTVRIPVSWHPHVS
GSDYKISDVWMNRVQEVVNYCIDNKMYVILNTHHDVDKVKGYFPSSQYMASSKKY
ITSVWAQIAARFANYDEHLIFEGMNEPRLVGHANEWWPELTNSDVVDSINCINQLNQ
DFVNTVRATGGKNASRYLMCPGYVASPDGATNDYFRMPNDISGNNNKIIVSVHAYC
PWNFAGLAMADGGTNAWNINDSKDQSEVTWFMDNIYNKYTSRGIPVIIGECGAVDK
NNLKTRVEYMSYYVAQAKARGILCILWDNNNFSGTGELFGFFDRRSCQFKFPEIIDG
MVKYAFEAKTDPDPVIVYGDYNNDGNVDALDFAGLKKYIMAADHAYVKNLDVNL
DNEVNAFDLAILKKYLLGMVSKLPSN

Beta-1,4-endoglucanase (EC 3.2.1.4) Ruminiclostridium josui (Clostridium josui)

MFRNTNKRILAFVIVVAMLMYFIPTMTFAVEEDSSHLITNQAKKPSTAGALQLLNKN
GVKTLCDKDGNPIQLRGMSTHGLQWFPEIINNNAFAALSKDWGSNVIRLAMYVAEG
GYSKDPEIIKKRVIDGIDLAIANDMYVIVDWHVLTPGDPNADVYKGAMDFFKEISQK
YPNNPHIIYELANEPSPNDPGVTNDAAGWAKVKSYAEPIIKILRDSGNKNLIIVGSPN
WSQRPDLAAENPINDNNTAYSFHFYSGTHKTSTDSTDRGNIMSNARYALEHGVAVF
CSEWGTSEASGNNGPYLKEADEWLEFLNANNISWINWSLTNKNETSGSFIPFISGKSD
ATNLNPGDDQVWSLKELSVSGEYARARIKGIKYEPIERSEKEEFTTNVWDFNDGTTQ
GFGINGDSPIKADSITLANEKNALKITGLNNSNDLTEGNYWANVRLSADGTSNKPNIF
GAEKLTMDVITAAPATVSIAAIPQSSTHGWANPTRAIAVKPADFVKQEDGTYKAVLT
ITPADSPNFDSIAKDSKDSTMTNIILFVGADTDVISLDNITVSGNRAVVEAPVEHAPIG
KATLPSTFEDSTRQGWAWDATSGVQSALTIKDANESKAISWEVKYPEVKPVDGWAS
APRIMLGNVNTTRGNNKYLTFDFYLKPTQASKGSLTISLAFAPPSLGFWAQATGDVN
IPLSSLSKMKKTTDGLYHFQVKYDLDKINDGKVLTANTVLRDITIVVADGNSDFAGT
MYLDNIRFENDSKTELNNSITMLVSKGIINNADVKKINFNSSISRGEFLMWIVKTLDL
NAKFSSNFSDVNKKGSYYNSVGIAKALGITSGVGNNKFNPNKAISREDMLVLTYKAL
KIVNKNLAKGNANDLKQFTDASKVSKNAVESVATIVKNGFYSGDAKKLNPKASVA
KSEAALMLYKIYSSLHK

Cellulase (EC 3.2.1.4) Ruminiclostridium papyrosolvens DSM 2782

MFKNMKKTISKVLVSSIIMSALFMVSAPAGVSAASDVTVNLGSTKQEIRGFGASSAW
CGTISDYVMNSLYGDLGYSILRLRIEEGIGDAWKTGNFSKWSPELANAKKASAKGAI
VFASPWNPPASMQENFSKSGDSSAQRLRYDKYTEYAQYLNAYVKYMKDNGVDLY
AISVQNEPDYAQDWTWWTPQEMLNFMKNNAGSINCRVMAPESFQFLKNMSDPILN
DATALDNMDVLGCHFYGTSVNNMAYPLYQQKSAGKELWMTEKYFDDDTTGNIMN
MSKEIHDSMVTGNMNAYIYWWITWPNGLATSSGTIYKRAYVLGQFAKFIRPGYKRV
DATATPNTNVYVSAYTGDNKAVIVAINTGTAAVSQKFNFQNGSASSVVSYVTDSSR
NMAAGANIAVINGSFTAQLPAQSITTFVGNTAPVVVEPIDAFNKIEAENYYDQSGTQ
TEANSDGNGKNVGYIENEDYLVFKNVDFGSGAASFEASAGSATNGGNIELRLDSLTG
TLIGNCAVPGTGGWQTWTNATCNVSQVTGKHDVYLKFTGESGYLMNLDWFKENT
KVIPVGKLGDINGDASIDSLDLMLIKKHLLGEAIENTALADLDGSGAVDAIDLAQMK
QYLLGIISAFPGKA

Endoglucanase 1 (EC 3.2.1.4) (Cellulase) (Endo-1,4-beta-glucanase) (Endoglucanase I) (EG-I) Ruminococcus albus

MNSKKIGAMIAAAVLSLIVMTPAATRKIVQRQTRNSSTAVENSAADESETENVPVSQ
THTNDTMTVTSAKDLVAKMTNGWNLGNTMDATAQGLGSEVSWLPLKVTTNKYMI
DMLPEAGFNVLRIPVSWGNHIIDDKYTSDPAWMDRVQEIVNYGIDNGLYVILNTHHE
EWYMPKPSEKDGDIEEIKAVWAQIADRFKGYDEHLIFEGLNEPRLRGEGAEWTGTSE
AREIINEYEKAFVETVRASGGNNGDRCLMITGYAASSAYNNLSAIELPEDSDKLIISVH
AYLPYSFALDTKGTDKYDPEDTAIPELFEHLNELFISKGIPVIVGEFGTMNKENTEDR
VKCLEDYLAAAAKYDIPCVWWDNYARIGNGENFGLMNRADLEWYFPDLIETFKTY
AEKDPASAE

Endoglucanase A (EC 3.2.1.4) (Cellulase A) (Endo-1,4-beta-glucanase A) (EGA) (Endo-1,4-beta-xylanase) (EC 3.2.1.8) Ruminococcus albus

MRKPDKDADRLTTLDLARSGEVRDISAMELVGEMKTGWNLGNSLDATGAPGNASE
VNWGNPKTTKEMIDAVYNKGFDVIRIPVTWGGHVGDAPDYKIDDEWIARVQEVVN
YAYDDGAYVIINSHHEEDWRIPDNEHIDAVDEKTAAIWKQVAERFKDYGDHLIFEGL
NEPRVKGSPQEWNGGTEEGRRCVDRLNKTFLDTVRATGGNNEKRLLLMTTYASSS
MSNVIKDTAIPEDDHIGFSIHAYTPYAFTYNANADWELFHWDDSHDGELVSLMTNL
KENYLDKDIPVIITEYGAVNKDNNDEDRAKWVSSYIEYAELLGGIPCVWWDNGYYS
SGNELFGIFDRNTCTWFTDTVTDAIIENAK

Endoglucanase (EC 3.2.1.4) Ruminococcus albus

MSKNAKIAVIAAFAVLATMMGMLFAKINALEAQNSAGGSPSVPAAAMQKNASQNN
ESSDSSNVTQNQDDEIVLDISYGESWGEMGEYYTNVTLNLKNTSDKAVSNWEVLLS
SAEGTRIEQIWNGEKSQEGNIIHITPVEYNKEIAPLAHLQAAPGMIVVSREKLLFESFS
AKAVIDGSDILIKSSENNISPQENNVVSLPAEQDLPISNGSGVSALGQLKVIGTKLCAE
DGSVVVLEGMSSHGLAWYPEFSAKYSVKKTKEYGANVYRAAMYTEEYGGYTTGE
NFRQEAEKILINAVDTAKSLDMYAIIDWHILSDGSPSKHKNEAIDFFDRISQKYANDP
AVIYEICNEPNGSDWSSDIKPYANEIIPVIRKNSPNAIVIVGTNTWSQDVDKASEDKLS
FDNIMYSFHFYAGTHKLDVFKQKIEKALANGCAVFVTEWGTSAADGNNGVYIKESG
EWLKYLSSKQISRINWSLSNKNESSAAVLPSASAENFSYEDLSESGKFVFDSFGNY

Endoglucanase (EC 3.2.1.4) Ruminococcus albus

MKESEIFINQIGYKPLDNKNVFVTNAAKGDAKEFTLCKKSDGQVVFTGALTAAPDDE
LSGGNYFTGDFSSIKTAGEYYVCVGQERSFDFKIADNVYDDVALSTLKYFTDSRCGQ
GICHTGIAEVYGTGEKKNVQGGWHDAGDYGRYIVAGTKTVMDMLLAYKKCPEKFA
NFDLLGEVRFELEWMLQMQRDDGAVYHKISCYHFCAFIMPQDEKDQLVLAPVSTA
ATADFAGCLAYASGFYKADDPDFAKKLLDAALKAQTYLFSHDDEFYINPPEITTGGY
GDRDVRDERYFALCSLFAATGDKNYLTQALKFREEKKKDKPDPKHPWNCGWQEGF
GWPFVAGYGTEILLEVAAENEENTALSPDLIAEIHTCIIAQAEKILEISNSSAFKYCSKL
VFWGSNGAICDLAHIMLMAYDLTGREDFFKAAKAQIDYILGCNPVNYCYVTGAGSK
PVVNPHHRPSGASGRVYPGMLSGGPCAGLLDAYAKEHLTGLPALKCFADAQPSYST
NEVAIYWNSAFVYLLARVR

Endoglucanase 4 (EC 3.2.1.4) (Cellulase 4) (Endo-1,4-beta-glucanase 4) (Endoglucanase IV) (EG-IV) Ruminococcus albus

MLDKLKVINGKLTAGEKPVRLFGLSTHGIAWYPEYICEESFNALKKDWRTNCIRIAM
YTDEFRGYCKDGNKQHLKELIEKGVVIAEKLDMYVIVDWHVLCDQDPMKYIDEAEE
FFSDMSKRFANKTNVIYEICNEPNCSGTWDKITEYADRIIPIIRSNSPDALIVTGTSTWS
QDIHCALEKPLKWDNVMYSLHFYAATHKGTLRSRLERCIEAGLPVFINEFNLCEASG
KGDIDIDEANAWYEVIDRLGLSCISWCLSNSGDTCGVFTQNCTKLSGWTDEDIKTSG
KIIKGWFEAFADEENTNEQCFRIDK

Endoglucanase 1 (EC 3.2.1.4) (Cellulase) (Endo-1,4-beta-glucanase) (Endoglucanase I) (EG-I) Ruminococcus albus

MNSKKIGAMIAAAVLSLIVMTPAATRKIVQRQTRNSSTAVENSAADESETENVPVSQ
THTNDTMTVTSAKDLVAKMTNGWNLGNTMDATAQGLGSEVSWLPLKVTTNKYMI
DMLPEAGFNVLRIPVSWGNHIIDDKYTSDPAWMDRVQEIVNYGIDNGLYVILNTHHE
EWYMPKPSEKDGDIEEIKAVWAQIADRFKGYDEHLIFEGLNEPRLRGEGAEWTGTSE
AREIINEYEKAFVETVRASGGNNGDRCLMITGYAASSAYNNLSAIELPEDSDKLIISVH
AYLPYSFALDTKGTDKYDPEDTAIPELFEHLNELFISKGIPVIVGEFGTMNKENTEDR
VKCLEDYLAAAAKYDIPCVWWDNYARIGNGENFGLMNRADLEWYFPDLIETFKTY
AEKDPASAE

Endoglucanase 5A (EC 3.2.1.4) (Alkaline cellulase) (Endo-1,4-beta-glucanase 5A) Salipaludibacillus agaradhaerens (Bacillus agaradhaerens)

MKKITTIFVVLLMTVALFSIGNTTAADNDSVVEEHGQLSISNGELVNERGEQVQLKG
MSSHGLQWYGQFVNYESMKWLRDDWGINVFRAAMYTSSGGYIDDPSVKEKVKEA
VEAAIDLDIYVIIDWHILSDNDPNIYKEEAKDFFDEMSELYGDYPNVIYEIANEPNGSD
VTWGNQIKPYAEEVIPIIRNNDPNNIIIVGTGTWSQDVHHAADNQLADPNVMYAFHF
YAGTHGQNLRDQVDYALDQGAAIFVSEWGTSAATGDGGVFLDEAQVWIDFMDER
NLSWANWSLTHKDESSAALMPGANPTGGWTEAELSPSGTFVREKIRESASIPPSDPTP
PSDPGEPDPTPPSDPGEYPAWDPNQIYTNEIVYHNGQLWQAKWWTQNQEPGDPYGP
WEPLN

Endoglucanase 5A (EC 3.2.1.4) (Alkaline cellulase) (Endo-1,4-beta-glucanase 5A) Salipaludibacillus agaradhaerens (Bacillus agaradhaerens)

MKKITTIFVVLLMTVALFSIGNTTAADNDSVVEEHGQLSISNGELVNERGEQVQLKG
MSSHGLQWYGQFVNYESMKWLRDDWGINVFRAAMYTSSGGYIDDPSVKEKVKEA
VEAAIDLDIYVIIDWHILSDNDPNIYKEEAKDFFDEMSELYGDYPNVIYEIANEPNGSD
VTWGNQIKPYAEEVIPIIRNNDPNNIIIVGTGTWSQDVHHAADNQLADPNVMYAFHF
YAGTHGQNLRDQVDYALDQGAAIFVSEWGTSAATGDGGVFLDEAQVWIDFMDER
NLSWANWSLTHKDESSAALMPGANPTGGWTEAELSPSGTFVREKIRESASIPPSDPTP
PSDPGEPDPTPPSDPGEYPAWDPNQIYTNEIVYHNGQLWQAKWWTQNQEPGDPYGP
WEPLN

Endoglucanase 1 (EC 3.2.1.4) (CEL1) (CMCase I) (Cellulase I) (Endo-1,4-beta-glucanase 1) Streptomyces halstedii

MSRKLRTLMAALCALPLAFAAAPPAHAADPTTMTNGFYADPDSSASRWAAANPGD
GRAAAINASIANTPMARWFGSWSGAIGTAAGAYAGAADGRDKLPILVAYNIYNRDY
CGGHSAGGAASPSAYADWIARFAGGIAARPAVVILEPDSLGDYGCMNPAQIDEREA
MLTNALVQFNRQAPNTWVYMDAGNPRWADAATMARRLHEAGLRQAHGFSLNVS
NYITTAENTAYGNAVNNELAARYGYTKPFVVDTSRNGNGSNGEWCNPSGRRIGTPT
RTGGGAEMLLWIKTPGESDGNCGVGSGSTAGQFLPEVAYKMIYGY

Endoglucanase CelA (EC 3.2.1.4) (Cellulase) (Endo-1,4-beta-glucanase) Streptomyces lividans

MKRLLALLATGVSIVGLTALAGPPAQAATGCKAEYTITSQWEGGFQAGVKITNLGD
PVSGWTLGFTMPDAGQRLVQGWNATWSQSGSAVTAGGVDWNRTLATGASADLGF
VGSFTGANPAPTSFTLNGATCSGSVTDPPTDPPTDPPATGTPAAVNGQLHVCGVHLC
NQYDRPIQLRGMSTHGIQWFGPCYGDASLDRLAQDWKSDLLRVAMYVQEDGYETD
PAGFTSRVNGLVDMAEDRGMYAVIDFHTLTPGDPNYNLDRARTFFSSVAARNDKKN
VIYEIANEPNGVSWTAVKSYAEQVIPVIRAADPDAVVIVGTRGWSSLGVSDGANESE
VVNNPVNATNIMYAFHFYAASHKDDYRAAVRPAATRLPLFVSEFGTVSATAWSVD
RSSSVAWLDLLDQLKISYANWTYSDADEGSAAFRPGTCEGTDYSSSGVLTESGALV
KSRISTTDDFPTS

Endoglucanase CelA (EC 3.2.1.4) (Cellulase) (Endo-1,4-beta-glucanase) Streptomyces lividans

MKRLLALLATGVSIVGLTALAGPPAQAATGCKAEYTITSQWEGGFQAGVKITNLGD
PVSGWTLGFTMPDAGQRLVQGWNATWSQSGSAVTAGGVDWNRTLATGASADLGF
VGSFTGANPAPTSFTLNGATCSGSVTDPPTDPPTDPPATGTPAAVNGQLHVCGVHLC
NQYDRPIQLRGMSTHGIQWFGPCYGDASLDRLAQDWKSDLLRVAMYVQEDGYETD
PAGFTSRVNGLVDMAEDRGMYAVIDFHTLTPGDPNYNLDRARTFFSSVAARNDKKN
VIYEIANEPNGVSWTAVKSYAEQVIPVIRAADPDAVVIVGTRGWSSLGVSDGANESE
VVNNPVNATNIMYAFHFYAASHKDDYRAAVRPAATRLPLFVSEFGTVSATAWSVD
RSSSVAWLDLLDQLKISYANWTYSDADEGSAAFRPGTCEGTDYSSSGVLTESGALV
KSRISTTDDFPTS

Cellulase 1 (EC 3.2.1.4) (Avicelase) (Endo-1,4-beta-glucanase) (Endoglucanase) Streptomyces reticuli

MKRRTTAVLTLTALLGTALTALPVQQAGAEEVEQVRNGTFDTTTDPWWTSNVTAG
LSDGRLCADVPGGTTNRWDSAIGQNDITLVKGETYRFSFHASGIPEGHVVRAVVGLA
VSPYDTWQEASPVLTEADGSYSYTFTAPVDTTQGQVAFQVGGSTDAWRFCVDDVSL
LGGVPPEVYEPDTGPRVRVNQVAYLPAGPKNATLVTDATARLPWQLRNAQGTTVA
RGLTVPRGVDASSGQNVHSIDFGSYRGRGTGYTLVADGETSHPFDIDAAAYRPLRLD
SVKYYYTQRSGIAIRDDLRPGYGRAAGHLNVAPNQGDANVPCQPGVCDYTLDVTG
GWYDAGDHGKYVVNGGIATWELLSTYERSLTARTGHPAALGDGTLALPESGNKVP
DVLDEARWELEFLLKMQVPAGQPLAGMAHHKLHDEQWTGLPLLPDQDPQKRELHP
PTTAATLNLAATAAQAARLYRPFDKAFAARALTAARTAWQAALAHPDLLADPNDG
TGGGAYNDDDVTDEFYWAAAELYLTTGERQFADHVLDSPVHTADIFGPTGFDWGH
TAAAGRLDLALVPSRLPGRDQVRRSVIKAADTYLATLTAHPYGMPYAPAGNRYDW
GSSHQVLNNGVVLASAYDLTGAAKYRDGALQGMDYVLGRNALNMSYVTGYGEVS
SHNQHSRWYAHQLDPTLPNPPSGTLAGGPNSSIQDPYAQSKLTGCVGQFCYIDDIQS
WSTNETAINWNAALARMASFAADQG

Cellulase 1 (EC 3.2.1.4) (Avicelase) (Endo-1,4-beta-glucanase) (Endoglucanase) Streptomyces reticuli

MKRRTTAVLTLTALLGTALTALPVQQAGAEEVEQVRNGTFDTTTDPWWTSNVTAG
LSDGRLCADVPGGTTNRWDSAIGQNDITLVKGETYRFSFHASGIPEGHVVRAVVGLA
VSPYDTWQEASPVLTEADGSYSYTFTAPVDTTQGQVAFQVGGSTDAWRFCVDDVSL
LGGVPPEVYEPDTGPRVRVNQVAYLPAGPKNATLVTDATARLPWQLRNAQGTTVA
RGLTVPRGVDASSGQNVHSIDFGSYRGRGTGYTLVADGETSHPFDIDAAAYRPLRLD
SVKYYYTQRSGIAIRDDLRPGYGRAAGHLNVAPNQGDANVPCQPGVCDYTLDVTG
GWYDAGDHGKYVVNGGIATWELLSTYERSLTARTGHPAALGDGTLALPESGNKVP
DVLDEARWELEFLLKMQVPAGQPLAGMAHHKLHDEQWTGLPLLPDQDPQKRELHP
PTTAATLNLAATAAQAARLYRPFDKAFAARALTAARTAWQAALAHPDLLADPNDG
TGGGAYNDDDVTDEFYWAAAELYLTTGERQFADHVLDSPVHTADIFGPTGFDWGH
TAAAGRLDLALVPSRLPGRDQVRRSVIKAADTYLATLTAHPYGMPYAPAGNRYDW
GSSHQVLNNGVVLASAYDLTGAAKYRDGALQGMDYVLGRNALNMSYVTGYGEVS
SHNQHSRWYAHQLDPTLPNPPSGTLAGGPNSSIQDPYAQSKLTGCVGQFCYIDDIQS
WSTNETAINWNAALARMASFAADQG

Endoglucanase 1 (EC 3.2.1.4) (CMCase I) (Carboxymethyl cellulase) (Cellulase) (Endo-1,4-beta-glucanase) Streptomyces sp. (strain KSM-9)

MENPRTTPTPTPLRRRRSERRARGGRVLTALTGVTLLAGLAIAPAATGASPSPAPPAS
PAPSADSGTADAGTTALPSMELYRAEAGVHAWLDANPGDHRAPLIAERIGSQPQAV
WFAGAYNPGTITQQVAEVTSAAAAAGQLPVVVPYMIPFRDCGNHSGGGAPSFAAYA
EWSGLFAAGLGSEPVVVVLEPDAIPLIDCLDNQQRAERLAALAGLAEAVTDANPEAR
VYYDVGHSAWHAPAAIAPTLVEAGILEHGAGIATNISNYRTTTDETAYASAVIAELG
GGLGAVVDTSRNGNGPLGSEWCDPPGRLVGNNPTVNPGVPGVDAFLWIKLPGELDG
CDGPVGSFSPAKAYELAGG

Endoglucanase (EC 3.2.1.4) Streptomyces sp. (strain SirexAA-E/ActE)

MRTAIRTARRPQPLALLLRGLAAFLGLALAGALGPATARAADLPQRAEARAAGLHIS
DGRLVEGNGNDFVMRGINHAHTWYPGETQSLADIKATGANTVRVVLSDGYRWSEN
SPEDVASIIARCKAERLICVLEVHDTTGYGEDAAAGTLDHAADYWIGLKDVLDGEED
YVVINIGNEPWGNADPAGWTAPTTAAIQKLRAAGFAHTIMVDAPNWGQDWEGVM
RADARSVYDADPTGNLIFSIHMYSVYDTAAKVTDYLNAFVDAGLPLLIGEFGGPADQ
YGDPDEDTMMATAEELGLGYLAWSWSGNTDPVLDLVLDFDPTRLSSWGERVLHGP
DGITETSREATVFGGGQGGGDTEAPTAPGTPTASGVTATSVTLGWSAATDDVGVTA
YDVVRVTGGSETKVASSAATSVTVTGLSAGTAYSFAVYARDAAGNRSARSGTVSVT
TDEGGSVPGGACSVGYRVIGEWPGGFQGEITLRNTGAAAVDGWTLGFAFADGQTVT
NMWGGTATQSGGAVSVTPASYTSTIAAGGSVTVGFTGTLTGANAAPAAFTLNGATC
TAA

Endoglucanase (EC 3.2.1.4) Streptomyces thermolilacinus

MRTAPPAPPGRSLRGLLAALVGLLVLAVGGAPAAARTAPPGSAGQPPDAAVTGLHV
QGGRLLEGNGNDFVMRGVNHAHTWYPGQTRSLADIKALGANTVRVVLSDGHRWT
RNGPADVAAVIDRCKANRLICVLEVHDTTGYGEEPAAGTLDHAADYWISLMDVLA
GQEDYVIVNIGNEPWGNTDPAGWTAPTIAAVKKLRAAGLAHTLMIDAPNWGQDWQ
GVMRADARSVYEADPTGNLLFSIHMYSVFDTAAEIDDYLEAFVDAGLPLVIGEFGGP
PDQWGDPDEDTMLAAAERLRLGYLAWSWSGNTDPVLDLAIGFDPDRLSGWGQRVF
HGVHGIGETSREATVFGGETPGDTRPPAAPGAPVASAVTAASATLTWPAATDDVGV
TGYEVVRVTGDTETRVAASTTPTATLTGLTAGTTYTFAVYARDAAGNRSPRSATVT
VTTDQGGGTPGTACSVAYRVVNEWPGGFQGEITLRNTGTAALGGWTLGFTFRDGQ
TITHMWGGTATRDGAEVTVAPAPYTSTVPAGGSVTLGFTGGRGTANTAPTAFTVNG
TPCAIG

Endoglucanase (EC 3.2.1.4) Streptomyces thermoluteus

MRTPDRTVPRAAALLAALAGLLLTLLVPTTPAHAAPTGFRVQNGRLLEASGNDFVM
RGVNHAHTWYADRLDALDHIKAKGANTVRVVLSSGDRWTRNDTADVANVVAQC
KRNRLICVLEVHDTTGYGEQSGAVTLSRAADYWIEVRDALAGQEDYVIVNIGNEPH
GNTDYTRWTADTKAAVQKLRAAGFGHTLMVDAPNWGQDWAFTMRDNAASVFAA
DPDANTVFSIHMYGVFDTAAEVSDYLGRFVAAKLPVVVGEFGHDHSDGNPDEDAIL
SVTRQLGIGYLGWSWSGNGGGVEYLDMVENFDPARTTSWGQRLFDGPDGIAATSEE
AAVYRGGPGEDTTAPTAPGAPAASAVTSSSVTLTWGAATDTVGVTGYDVVRVAGG
QETVVASSSDTTATVTGLAADTSYTFAVYARDAAGNRSARSATATVTTASGPAAGS
CAVTYRVTGEWPGGFQGEVVVRNTGSTAVDGWTLRWTFPDGQRITSLWGGTVTQS
GAEVSVAAAPYTATIPPSGAVSLGFTGSRAGTNTDPTVFLLNGAECSAA

Cellulase (EC 3.2.1.4) Teredinibacter turnerae (strain ATCC 39867/T7901)

MFPRLSPSRFRQVTLTLLTLGLVSLTGCAGNSKPDADTSTAGAVATGEYRNLFAEIG
KSEIDIQRKIDEAFQHLFYGDAKDAAVYYQAGGNENGPLAYVYDVNSNDVRSEGMS
YGMMITVQMDKKAEFDAIWNWAKTYMYQDSPTHPAFGYFAWSMRRDGVANDDM
PAPDGEEYFVTALYFAAARWGNGEGIFNYQQEADTILSRMRHRQVITGPTNRGVMT
ATNLFHPEEAQVRFTPDINNADHTDASYHLPSFYEIWARVAPQEDRAFWAKAADVS
RDYFAKAAHPVTALTPDYGNFDGTPWAASWRPESVDFRYDAWRSVMNWSMDYA
WWGKDSGAPARSDKLLAFFETQEGKMNHLYSLDGKPLGGGPTLGLISMNATAAMA
ATDPRWHNFVEKLWQQQPPTGQYRYYDGVLYLMALLHCAGEYKAWIPDGE

Cellulase Ce1DZ1 (EC 3.2.1.4) Thermoanaerobacterium sp

MNKWHINKWYFFVGMLVIFAVIISLILKDTSLTFSSYDREKFPHLIGNSMVKKPSLAG
RLKIIEIDGRKTLGDQHGNPIQLRGMSTHGLQWFPQIINNNAFSALSKDWEANVIRLA
MYVGEGGYSTDPSVKEKVIEGINLAIKNDMYVIVDWHILNPGDPNAKIYSGAKEFFK
EIASKYPNDLHIIYELANEPNPTESDITNDIAGWEKVKKYAEPIIKMLRDMGNENIIIVG
NPEWSTRPDLAVNDPIDDKNVMYSAHFYTGSASVWENGNKGHIARNIEKALENGLT
VFVTEWGTSEASGDGGPYLNEADEWLEFLNSNNISWVNWSLANKNEASAAFLPTTS
LDPGNGKVWAVNQLSLSGEYVRARIKGIPYKPISRETMGK

Cellulase Ce1DZ1 (EC 3.2.1.4) Thermoanaerobacterium sp

MNKWHINKWYFFVGMLVIFAVIISLILKDTSLTFSSYDREKFPHLIGNSMVKKPSLAG
RLKIIEIDGRKTLGDQHGNPIQLRGMSTHGLQWFPQIINNNAFSALSKDWEANVIRLA
MYVGEGGYSTDPSVKEKVIEGINLAIKNDMYVIVDWHILNPGDPNAKIYSGAKEFFK
EIASKYPNDLHIIYELANEPNPTESDITNDIAGWEKVKKYAEPIIKMLRDMGNENIIIVG
NPEWSTRPDLAVNDPIDDKNVMYSAHFYTGSASVWENGNKGHIARNIEKALENGLT
VFVTEWGTSEASGDGGPYLNEADEWLEFLNSNNISWVNWSLANKNEASAAFLPTTS
LDPGNGKVWAVNQLSLSGEYVRARIKGIPYKPISRETMGK

Endoglucanase E-2 (EC 3.2.1.4) (Cellulase E-2) (Cellulase E2) (Endo-1,4-beta-glucanase E-2) Thermobifida fusca (Thermomonospora fusca)

MSPRPLRALLGAAAAALVSAAALAFPSQAAANDSPFYVNPNMSSAEWVRNNPNDP
RTPVIRDRIASVPQGTWFAHHNPGQITGQVDALMSAAQAAGKIPILVVYNAPGRDCG
NHSSGGAPSHSAYRSWIDEFAAGLKNRPAYIIVEPDLISLMSSCMQHVQQEVLETMA
YAGKALKAGSSQARIYFDAGHSAWHSPAQMASWLQQADISNSAHGIATNTSNYRW
TADEVAYAKAVLSAIGNPSLRAVIDTSRNGNGPAGNEWCDPSGRAIGTPSTTNTGDP
MIDAFLWIKLPGEADGCIAGAGQFVPQAAYEMAIAAGGTNPNPNPNPTPTPTPTPTPP
PGSSGACTATYTIANEWNDGFQATVTVTANQNITGWTVTWTFTDGQTITNAWNAD
VSTSGSSVTARNVGHNGTLSQGASTEFGFVGSKGNSNSVPTLTCAAS

Endoglucanase E-4 (EC 3.2.1.4) (Cellulase E-4) (Cellulase E4) (Endo-1,4-beta-glucanase E-4) Thermobifida fusca (Thermomonospora fusca)

MSVTEPPPRRRGRHSRARRFLTSLGATAALTAGMLGVPLATGTAHAEPAFNYAEAL
QKSMFFYEAQRSGKLPENNRVSWRGDSGLNDGADVGLDLTGGWYDAGDHVKFGF
PMAFTATMLAWGAIESPEGYIRSGQMPYLKDNLRWVNDYFIKAHPSPNVLYVQVGD
GDADHKWWGPAEVMPMERPSFKVDPSCPGSDVAAETAAAMAASSIVFADDDPAYA
ATLVQHAKQLYTFADTYRGVYSDCVPAGAFYNSWSGYQDELVWGAYWLYKATGD
DSYLAKAEYEYDFLSTEQQTDLRSYRWTIAWDDKSYGTYVLLAKETGKQKYIDDAN
RWLDYWTVGVNGQRVPYSPGGMAVLDTWGALRYAANTAFVALVYAKVIDDPVR
KQRYHDFAVRQINYALGDNPRNSSYVVGFGNNPPRNPHHRTAHGSWTDSIASPAEN
RHVLYGALVGGPGSPNDAYTDDRQDYVANEVATDYNAGFSSALAMLVEEYGGTPL
ADFPPTEEPDGPEIFVEAQINTPGTTFTEIKAMIRNQSGWPARMLDKGTFRYWFTLDE
GVDPADITVSSAYNQCATPEDVHHVSGDLYYVEIDCTGEKIFPGGQSEHRREVQFRIA
GGPGWDPSNDWSFQGIGNELAPAPYIVLYDDGVPVWGTAPEEGEEPGGGEGPGGGE
EPGEDVTPPSAPGSPAVRDVTSTSAVLTWSASSDTGGSGVAGYDVFLRAGTGQEQK
VGSTTRTSFTLTGLEPDTTYIAAVVARDNAGNVSQRSTVSFTTLAENGGGPDASCTV
GYSTNDWDSGFTASIRITYHGTAPLSSWELSFTFPAGQQVTHGWNATWRQDGAAVT
ATPMSWNSSLAPGATVEVGFNGSWSGSNTPPTDFTLNGEPCALA

Endoglucanase E-5 (EC 3.2.1.4) (Cellulase E-5) (Cellulase E5) (Endo-1,4-beta-glucanase E-4) Thermobifida fusca (Thermomonospora fusca)

MAKSPAARKGXPPVAVAVTAALALLIALLSPGVAQAAGLTATVTKESSWDNGYSAS
VTVRNDTSSTVSQWEVVLTLPGGTTVAQVWNAQHTSSGNSHTFTGVSWNSTIPPGG
TASSGFIASGSGEPTHCTINGAPCDEGSEPGGPGGPGTPSPDPGTQPGTGTPVERYGK
VQVCGTQLCDEHGNPVQLRGMSTHGIQWFDHCLTDSSLDALAYDWKADIIRLSMYI
QEDGYETNPRGFTDRMHQLIDMATARGLYVIVDWHILTPGDPHYNLDRAKTFFAEI
AQRHASKTNVLYEIANEPNGVSWASIKSYAEEVIPVIRQRDPDSVIIVGTRGWSSLGV
SEGSGPAEIAANPVNASNIMYAFHFYAASHRDNYLNALREASELFPVFVTEFGTETYT
GDGANDFQMADRYIDLMAERKIGWTKWNYSDDFRSGAVFQPGTCASGGPWSGSSL
KASGQWVRSKLQS

Endoglucanase E-2 (EC 3.2.1.4) (Cellulase E-2) (Cellulase E2) (Endo-1,4-beta-glucanase E-2) Thermobifida fusca (Thermomonospora fusca)

MSPRPLRALLGAAAAALVSAAALAFPSQAAANDSPFYVNPNMSSAEWVRNNPNDP
RTPVIRDRIASVPQGTWFAHHNPGQITGQVDALMSAAQAAGKIPILVVYNAPGRDCG
NHSSGGAPSHSAYRSWIDEFAAGLKNRPAYIIVEPDLISLMSSCMQHVQQEVLETMA
YAGKALKAGSSQARIYFDAGHSAWHSPAQMASWLQQADISNSAHGIATNTSNYRW
TADEVAYAKAVLSAIGNPSLRAVIDTSRNGNGPAGNEWCDPSGRAIGTPSTTNTGDP
MIDAFLWIKLPGEADGCIAGAGQFVPQAAYEMAIAAGGTNPNPNPNPTPTPTPTPTPP
PGSSGACTATYTIANEWNDGFQATVTVTANQNITGWTVTWTFTDGQTITNAWNAD
VSTSGSSVTARNVGHNGTLSQGASTEFGFVGSKGNSNSVPTLTCAAS

Endoglucanase E-4 (EC 3.2.1.4) (Cellulase E-4) (Cellulase E4) (Endo-1,4-beta-glucanase E-4) Thermobifida fusca (Thermomonospora fusca)

MSVTEPPPRRRGRHSRARRFLTSLGATAALTAGMLGVPLATGTAHAEPAFNYAEAL
QKSMFFYEAQRSGKLPENNRVSWRGDSGLNDGADVGLDLTGGWYDAGDHVKFGF
PMAFTATMLAWGAIESPEGYIRSGQMPYLKDNLRWVNDYFIKAHPSPNVLYVQVGD
GDADHKWWGPAEVMPMERPSFKVDPSCPGSDVAAETAAAMAASSIVFADDDPAYA
ATLVQHAKQLYTFADTYRGVYSDCVPAGAFYNSWSGYQDELVWGAYWLYKATGD
DSYLAKAEYEYDFLSTEQQTDLRSYRWTIAWDDKSYGTYVLLAKETGKQKYIDDAN
RWLDYWTVGVNGQRVPYSPGGMAVLDTWGALRYAANTAFVALVYAKVIDDPVR
KQRYHDFAVRQINYALGDNPRNSSYVVGFGNNPPRNPHHRTAHGSWTDSIASPAEN
RHVLYGALVGGPGSPNDAYTDDRQDYVANEVATDYNAGFSSALAMLVEEYGGTPL
ADFPPTEEPDGPEIFVEAQINTPGTTFTEIKAMIRNQSGWPARMLDKGTFRYWFTLDE
GVDPADITVSSAYNQCATPEDVHHVSGDLYYVEIDCTGEKIFPGGQSEHRREVQFRIA
GGPGWDPSNDWSFQGIGNELAPAPYIVLYDDGVPVWGTAPEEGEEPGGGEGPGGGE
EPGEDVTPPSAPGSPAVRDVTSTSAVLTWSASSDTGGSGVAGYDVFLRAGTGQEQK
VGSTTRTSFTLTGLEPDTTYIAAVVARDNAGNVSQRSTVSFTTLAENGGGPDASCTV
GYSTNDWDSGFTASIRITYHGTAPLSSWELSFTFPAGQQVTHGWNATWRQDGAAVT
ATPMSWNSSLAPGATVEVGFNGSWSGSNTPPTDFTLNGEPCALA

Endoglucanase E-5 (EC 3.2.1.4) (Cellulase E-5) (Cellulase E5) (Endo-1,4-beta-glucanase E-4) Thermobifida fusca (Thermomonospora fusca)

MAKSPAARKGXPPVAVAVTAALALLIALLSPGVAQAAGLTATVTKESSWDNGYSAS
VTVRNDTSSTVSQWEVVLTLPGGTTVAQVWNAQHTSSGNSHTFTGVSWNSTIPPGG
TASSGFIASGSGEPTHCTINGAPCDEGSEPGGPGGPGTPSPDPGTQPGTGTPVERYGK
VQVCGTQLCDEHGNPVQLRGMSTHGIQWFDHCLTDSSLDALAYDWKADIIRLSMYI
QEDGYETNPRGFTDRMHQLIDMATARGLYVIVDWHILTPGDPHYNLDRAKTFFAEI
AQRHASKTNVLYEIANEPNGVSWASIKSYAEEVIPVIRQRDPDSVIIVGTRGWSSLGV
SEGSGPAEIAANPVNASNIMYAFHFYAASHRDNYLNALREASELFPVFVTEFGTETYT
GDGANDFQMADRYIDLMAERKIGWTKWNYSDDFRSGAVFQPGTCASGGPWSGSSL
KASGQWVRSKLQS

Thermostable celloxylanase (EC 3.2.1.4) (EC 3.2.1.8) Thermoclostridium stercorarium (Clostridium stercorarium)

MNKFLNKKWSLILTMGGIFLMATLSLIFATGKKAFNDQTSAEDIPSLAEAFRDYFPIG
AAIEPGYTTGQIAELYKKHVNMLVAENAMKPASLQPTEGNFQWADADRIVQFAKEN
GMELRFHTLVWHNQTPTGFSLDKEGKPMVEETDPQKREENRKLLLQRLENYIRAVV
LRYKDDIKSWDVVNEVIEPNDPGGMRNSPWYQITGTEYIEVAFRATREAGGSDIKLY
INDYNTDDPVKRDILYELVKNLLEKGVPIDGVGHQTHIDIYNPPVERIIESIKKFAGLG
LDNIITELDMSIYSWNDRSDYGDSIPDYILTLQAKRYQELFDALKENKDIVSAVVFWG
ISDKYSWLNGFPVKRTNAPLLFDRNFMPKPAFWAIVDPSRLRE

Thermostable celloxylanase (EC 3.2.1.4) (EC 3.2.1.8) Thermoclostridium stercorarium (Clostridium stercorarium)

MNKFLNKKWSLILTMGGIFLMATLSLIFATGKKAFNDQTSAEDIPSLAEAFRDYFPIG
AAIEPGYTTGQIAELYKKHVNMLVAENAMKPASLQPTEGNFQWADADRIVQFAKEN
GMELRFHTLVWHNQTPTGFSLDKEGKPMVEETDPQKREENRKLLLQRLENYIRAVV
LRYKDDIKSWDVVNEVIEPNDPGGMRNSPWYQITGTEYIEVAFRATREAGGSDIKLY
INDYNTDDPVKRDILYELVKNLLEKGVPIDGVGHQTHIDIYNPPVERIIESIKKFAGLG
LDNIITELDMSIYSWNDRSDYGDSIPDYILTLQAKRYQELFDALKENKDIVSAVVFWG
ISDKYSWLNGFPVKRTNAPLLFDRNFMPKPAFWAIVDPSRLRE

Cellulase (EC 3.2.1.4)Thermococcus sp. 2319x1

MNRQKALAMFVLFFVLVGVIGSIPASHATTDTSTYTTPTGIYYEVRGDTIYMINPSSG
EEVPIHLFGVNWFGFETPNYVVHGLWSRNWEDMLLQIKSLGFNAIRLPFCTQSVKPG
TMPTGIDYAKNPDLQGLDSVQIMEKIIKKAGDLGIFVLLDYHRIGCNFIEPLWYTDSF
SEQDYINTWVEVAQRFGKYWNVIGADLKNEPHSSSPAPAAYTDGSGATWGMGNNA
TDWNLAAERIGKAILEVAPHWLIFVEGTQFTTPEIDGSYEWGHNAWWGGNLMGVR
EYPVNLPRNKLVYSPHVYGPDVYDQPYFDPAEGFPDNLPDIWYHHFGYVKLDLGYP
VVIGEFGGKYGHGGDARDVTWQNKIIDWMIQNKFCDFFYWSWNPNSGDTGGILQD
DWTTIWEDKYNNLKRLMDSCSGNAIAPPVPTTTTSTTTTTTTQTPTTSTPTTTTTTTTT
TTTTPPGSIPFETVNILPTSSQYEGTSVEVVCDGTQCASSVWGAPNLWGVVKIGNAT
MDPNVWGWEDVYRTAPQDIGTGSTKMEIGNGVLKVTSLWNINMHPKYNTMAYHE
VIYGAKPWGNQPINAQNFVLPIQVSQLPRILVDTKYTLEKSFPGNNFAFEAWLFKDT
DNMRAPGQGDYEIMVQLYIEGGYPAGYDKGPVLTVDVPIIVDGKLVNQTFELYDVV
ADAGWRFLTFKSTKNYNGSEVVFDYTKFIEIVDGYLSGGNLTNHYLMSLEFGTEVYT
NGCSSFPCTVDVRWTLDKYRFILAPNTMTTEEAMSVLIGEVQPPASTTTSQTTTSTPT
PTTTTTAPPTTTPPAQDVIKIRYPDDGQWPEAPIDGDGDGNPEFYIEINPWNIQSAEGY
AEMTYNLSTGVLHYVQALDDITLKNGGSWVHGYPEIFYGNKPWNNNYATDGEVPL
PGKVSNLSNFYLSVSYKLLPKNGLPINFAIESWLTREPWRNSGINSDEQELMIWLYYD
GLQPAGSKVKEIIVPIVVNGTPVNATFEVWKANIGWEYIAFRIKTPIKEGTVTIPYGAF
ISAAANVTSLANYTELYLEDVEVGTEYGTPSTTSAHLEWWFYNVSLEYRPGEPLLSQ
PPAEGSAPSEGGQTPSEGGATTGTLDAKLVNSWGTGAQYEVAVNLDTSSTWKLLIKI
KDGKISDIWGATIVGTQGDYVVVQPSSPAASATVGFVTSGNAPLVEEAVLLSGDKVL
AMWTAPTASASDLNVTIKIDSEWDSGFVVKIYVTNNGNAPVSNWQIKLRMTSLISGI
WGGTYTVSGDVVTIVPTGSNAVINPGTTIEIGFVASKQGAYVYPELLGVEIL

Endoglucanase (EC 3.2.1.4) Thermogutta terrifontis

MWNIFAIMSIAVACAAACAQEPVTVLRWDFEEIVGEQNVQALPAEWIASSKEGIAVE
RTEQGGRCLHVWVDAATGPGSRNIRYRLPVDKLRGQRVRVNALVRAKGVSQPPKP
WNGIKCMLRIESGGEIQWPQQNLPGGDFDWRPIQFVVAVPDDCQQVDLIVGLENVT
GDAWFDNIAVEVIPKKKLSSNHKEVFKGHNLPRLRGAMIGPHVTNADLLEFGNVWK
ANHIRWQLIWNGFPHSPADSATLDEYRQWLDGALKRLEAALPVCREAGILVTVDLH
TPPGGRNEASECRIFHDREFQKAFIDIWEDIARRFADSDVVWGYDLVNEPVEGMVPD
GLMNWQRLAEETARRVRAIDQKHAIIIEPAPWGSPSSIALLDPIDVPGVVYSVHMYVP
HAFTHQGVYDNPVGIVYPGTIDGKWYDRNTLRKVLEPVRRFQEENGVHIYIGEFSAI
RWAPADSACQYLKDCIEIFEEYGWDWAYHAFREWDGWSVEHGPDRNDRNRTATPT
DRALLLRSWYAENVKPQFSDKKKD

Endo-1,4-beta-glucanase (EC 3.2.1.4) Thermotoga maritima

MVLMTKPGTSDFVWNGIPLSMELNLWNIKEYSGSVAMKFDGEKITFDADIQNLSPKE
PERYVLGYPEFYYGYKPWENHTAEGSKLPVPVSSMKSFSVEVSFDIHHEPSLPLNFA
METWLTREKYQTEASIGDVEIMVWFYFNNLTPGGEKIEEFTIPFVLNGESVEGTWEL
WLAEWGWDYLAFRLKDPVKKGRVKFDVRHFLDAAGKALSSSARVKDFEDLYFTV
WEIGTEFGSPETKSAQFGWKFENFSIDLEVRE

Endoglucanase M (EC 3.2.1.4) Thermotoga maritima (strain ATCC 43589/DSM 3109/JCM 10099/NBRC 100826/MSB8)

MKELIRKLTEAFGPSGREEEVRSIILEELEGHIDGHRIDGLGNLIVWKGSGEKKVILDA
HIDEIGVVVTNVDDKGFLTIEPVGGVSPYMLLGKRIRFENGTIGVVGMEGETTEERQE
NVRKLSFDKLFIDIGANSREEAQKMCPIGSFGVYDSGFVEVSGKYVSKAMDDRIGCA
VIVEVFKRIKPAVTLYGVFSVQEEVGLVGASVAGYGVPADEAIAIDVTDSADTPKAIK
RHAMRLSGGPALKVKDRASISSKRILENLIEIAEKFDIKYQMEVLTFGGTNAMGYQR
TREGIPSATVSIPTRYVHSPSEMIAPDDVEATVDLLIRYLGA

Major extracellular endoglucanase (EC 3.2.1.4) (Cellulase) (Endo-1,4-beta-glucanase) Xanthomonas campestris pv. campestris (strain ATCC 33913/DSM 3586/NCPPB 528/LM 568/P 25

MSIFRTASTLALATALALAAGPAFSYSINNSRQIVDDSGKVVQLKGVNVFGFETGNH
VMHGLWARNWKDMIVQMQGLGFNAVRLPFCPATLRSDTMPASIDYSRNADLQGLT
SLQILDKVIAEFNARGMYVLLDHHTPDCAGISELWYTGSYTEAQWLADLRFVANRY
KNVPYVLGLDLKNEPHGAATWGTGNAATDWNKAAERGSAAVLAVAPKWLIAVEG
ITDNPVCSTNGGIFWGGNLQPLACTPLNIPANRLLLAPHVYGPDVFVQSYFNDSNFPN
NMPAIWERHFGQFAGTHALLLGEFGGKYGEGDARDKTWQDALVKYLRSKGINQGF
YWSWNPNSGDTGGILRDDWTSVRQDKMTLLRTLWGTAGNTTPTPTPTPTPTPTPTPT
PTPTPTPGTSTFSTKVIVDNSWNGGYCNRVQVTNTGTASGTWSIAVPVTGTVNNAW
NATWSQSGSTLRASGVDENRTLAAGATAEFGFCAAS

Major extracellular endoglucanase (EC 3.2.1.4) (Cellulase) (Endo-1,4-beta-glucanase) Xanthomonas campestris pv. campestris (strain ATCC 33913/DSM 3586/NCPPB 528/LMG 568/P 25)

MSIFRTASTLALATALALAAGPAFSYSINNSRQIVDDSGKVVQLKGVNVFGFETGNH
VMHGLWARNWKDMIVQMQGLGFNAVRLPFCPATLRSDTMPASIDYSRNADLQGLT
SLQILDKVIAEFNARGMYVLLDHHTPDCAGISELWYTGSYTEAQWLADLRFVANRY
KNVPYVLGLDLKNEPHGAATWGTGNAATDWNKAAERGSAAVLAVAPKWLIAVEG
ITDNPVCSTNGGIFWGGNLQPLACTPLNIPANRLLLAPHVYGPDVFVQSYFNDSNFPN
NMPAIWERHFGQFAGTHALLLGEFGGKYGEGDARDKTWQDALVKYLRSKGINQGF
YWSWNPNSGDTGGILRDDWTSVRQDKMTLLRTLWGTAGNTTPTPTPTPTPTPTPTPT
PTPTPTPGTSTFSTKVIVDNSWNGGYCNRVQVTNTGTASGTWSIAVPVTGTVNNAW
NATWSQSGSTLRASGVDFNRTLAAGATAEFGFCAAS

Endoglucanase, family GH5 (EC 3.2.1.4) Zobellia galactanivorans (strain DSM 12802/CCUG 47099/CIP 106680/NCIMB 13871/Dsij)

MRKTVLIFMVLSVNFSLFTSCGEKEHSDGAHVSGDPDIREMSKEEGNEEDGEDDGN
MREIAPKEFVLDMGAGWNLGNAMDTYNSDETAWGNPLTTKAMIDEIAKMGFKTLR
LPVTWKFHIGEGPDYLIEANWLDKVEAIANFALENEMYVIINIHHDETWILPTYEKAD
EVKDELSKVWTQIANRFKTYGDYLIFETLNEPRHKGTPEEWKGGTQEGRDAVNQYH
QVSVDAIRATGGNNAKRKIMVSTYAASTASNALNDYLVPNGDKNVIVSVHSYFPYQ
FCLDGTDSTWGTEADKTALLAELDKIRDKFIVEDNRAVVMGEWGSTFSDNPEDRLA
HAEFYARACAERGICPIWWDNGNVDEFGIFNRNTLEWNYPEIAEAIVKETTEARSKA
KTE

Suitable exogenous or heterologous nucleic acid sequences encoding a protein with cellulase activity may also include nucleic acid sequences which can be derived as a result of the degenerate genetic code from the amino acid sequences identified above as well as functional equivalents with cellulase activity.

Suitable exogenous or heterologous nucleic acid sequences that that encode a protein with glucosidase activity, carbohydrase activity, glucanase activity, galactosidase activity, fructofuranosidase activity, mannanase activity, mannosidase activity, thioglucosidase activity, maltase activity, lactase activity, sucrase activity, hexosanase activity, pentosanase activity, fructosyltransferase activity, chitinase activity, or pectinase activity.

For the purposes of the present disclosure, heterologous carbohydrases and the transgenes that express them can be derived from a variety of organisms that natively express such enzymes, such as certain bacteria, cyanobacteria, fungi, algae, and other microbes or animals.

In certain aspects, the C. necator may be engineered to express one or more functional fragments of a carbohydrase. That is, the functional fragment is a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length polypeptide. The functional fragment can generally, but not always, be comprised of a continuous region of the protein, wherein the region has functioning carbohydrase activity.

An engineered strain of C. necator can express a carbohydrase alone or in combination with one or more of digestive enzyme, including but not limited to, other carbohydrase enzymes, a protease, a lipase, and/or a phytase.

### D. Lipase Enzymes

C. necator does not naturally produce significant amounts of digestive enzymes exhibiting lipase activity. Accordingly, the present disclosure provides for the introduction of nucleic acids encoding a lipase enzyme. Thus, the engineered C. necator of the present disclosure may be modified to express a lipase enzyme. Accordingly, various carbohydrase enzymes may be used in combination with the above-disclosed proteases and carbohydrases to provide a further engineered biosynthesis of digestive enzymes.

Suitable exogenous or heterologous nucleic acid sequences that encode a protein with lipase activity include, but are not limited to, a nucleic acid sequences that code for proteins or polypeptides having lipase activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Acinetobacter baumannii (strain AYE) Lipase:
MKKTATALTLACSLLSVMSISQAQAADNIDVSFQTILQQERNWAGLQSKSLKVGDIT
WSYSEGGSSTKPTLLLIHGLAGSRDNWNRVAHYLTTNYHVIIPDLPGSGETIVSQDFD
YSVPNLAEKLRRFVEAANLKGPIHIAGHSLGGSIALLYAGQYPFETKSLFLVDSGGIFR
SANTIYLKDPTYLKQLLVSKKGDFNYLLKQTMFNPPFIPKEFLQAQEKLMINQAPQT
QKLVDQLIALNKVYTPDSFAVLTKTIDAPTLILWGKQDKIINVEVANELKRLLKNAQP
PVILENVGHMPILEAEQLVIQQYVPFLLKVETNQSSKTTTP
Aeribacillus pallidus Lipase:
MAVESRRPSVIRPGLFSVEAAADRRRREFDHHNEAILHKQVPIDVVFFGDSITHWWD
VSTYFGRSGRVVVNRGIGGDTSEYARRRFAADVLQLKPKYAVILIGVNNTWALDAW
HPEERRTAEQICEEVVADVESMLKAAHEHGIVPILCSILPTRIDRYARNEERNRLIVRI
NGGLKDLAGRLNVIYVDYHTALADADGVTLQEGLADDGLHPHVLGYDIMAAVLRE
TLAGHGIDL
Aeromonas hydrophila Lipase:
MKKKLIYAAVVSALLAGCGGSDDNKGDTSSYLDYLLTGSNAVGPSALAARAWDGT
LKFSTETADLSNPVSAMSTLDGWSTTQAIQIVPVTSSGITVQAPTTAEFGASVAPLYL
LEVTFDSTALRPSGVKKVLTYGVDFVVAASAWQAEPGSAQAVEPLPCLANDSGHRT
AERQSRRCLKAGSDYGNYKNNAGSNAQEQTINGLIALQEGLFKAATGIATDHVIFSD
WFGTQSGADVLVAVKGAAASVLKADPVTLDAAKLWKQDAWEHQPARHLYPGRD
RPTCLPDPAGCRAVPAAEQKDAIATAFGPVLRSTRLLKRPRSIPVPSSCLTSSPHRRPQ
VPGARPRPSPGTVPSQPVRHRQCAEGVTRSDRRAGGGGRGSGPAGDADCRSDPPER
AAGRGEQADWGDAHLRRQAAGRRAEHWSLQPAADAGRGAIRADACLRQGCPQHH
HGCHHLSARRDLGQRERLRPGAGPDLEDLCRHAGGQEGGAGGDRSSAARRAWLRL
SGSMDTVTTSDNPTPYLNLSYLTVARDNLKQSVAICWACVWRLAWPTPRAIGTAGS
LKVHFLGHSLGASRVPTCCGRQPDHRQRASGCPVQVRYRWPGHAGSHSAAAAELA
DFGPTIKMGVLTSGSAELKAGFTAYAPNCTDGGAYLLRQRVPAEPGRGHSATAATR
CRVQLCGPVGAGFG
Bacillus subtilis (strain 168) Lipase EstA:
MKFVKRRIIALVTILMLSVTSLFALQPSAKAAEHNPVVMVHGIGGASFNFAGIKSYLV
SQGWSRDKLYAVDFWDKTGTNYNNGPVLSRFVQKVLDETGAKKVDIVAHSMGGA
NTLYYIKNLDGGNKVANVVTLGGANRLTTGKALPGTDPNQKILYTSIYSSADMIVM
NYLSRLDGARNVQIHGVGHIGLLYSSQVNSLIKEGLNGGGQNTN
Burkholderia cepacia (Pseudomonas cepacia) Triacylglycerol lipase:
MARTMRSRVVAGAVACAMSIAPFAGTTAVMTLATTHAAMAATAPAAGYAATRYPI
ILVHGLSGTDKYAGVLEYWYGIQEDLQQNGATVYVANLSGFQSDDGPNGRGEQLL
AYVKTVLAATGATKVNLVGHSQGGLSSRYVAAVAPDLVASVTTIGTPHRGSEFADF
VQDVLAYDPTGLSSSVIAAFVNVFGILTSSSHNTNQDALAALQTLTTARAATYNQNY
PSAGLGAPGSCQTGAPTETVGGNTHLLYSWAGTAIQPTLSVFGVTGATDTSTLPLVD
PANVLDLSTLALFGTGTVMINRGSGQNDGLVSKCSALYGKVLSTSYKWNHLDEINQ
LLGVRGAYAEDPVAVIRTHANRLKLAGV
Clostridium novyi Liposomase:
MQSKLKKFISTISVSLFTLASVLTFSMAKPVNAFAKENQKVSQNNYPIILCHGCNGW
GRTENFGTVAFGARYYWGGNVDLQQKLIENGFTTYTAAVGPLSSNWDRACELYAQI
KGGRVDYGEAHAKKHGHSRYGRYYRGFYPEWGTKDENGNIRKVHLIGHSQGGQTI
RMLTTLLGQGKQEEISATGENTNELFKGGHDWVSGLITLASPHDGTTLADMKGTDA
AAAIGIGAVGSMLGNISNSDIIFDLKVDQWGLKRKPSESFISYFNRCATSKMWNSKDI
CSVDLSTNGAVEQNKWVKAQPNVYYFSWACCGTIRNPISIFGHHMASPIRMSDKGL
YNVQWALQAQLMGTYSRNNPRRAIPVIDSKWWPNDGYVNTISENGPKAGSTDEIVN
YNGTPQIGKWNFMGVKDMDHEDIIGRHWNGAPNFFIDMAQMLRNLPVTE
Geobacillus stearothermophilus (Bacillus stearothermophilus) Lipase:
MMKGCRVMVVLLGLWFVFGLSVPGGRTEAASPRANDAPIVLLHGFTGWGREEMLG
FKYWGGVRGDIEQWLNDNGYRTYTLAVGPLSSNWDRACEAYAQLVGGTVDYGAA
HAANDGHARFGRTYPGLLPELKRGGRVHIIAHSQGGQTARMLVSLLENGSQEEREY
AKEHNVSLSPLFEGGHRFVLSVTTIATPHDGTTLVNMVDFTDRFFDLQKAVLEAAAV
ASNAPYTSEIYDFKLDQWGLRREPGESFDHYFERLKRSPVWTSTDTARYDLSVPGAE
TLNRWVKASPNTYYLSFSTERTYRGALTGNYYPELGMNAFSAIVCAPFLGSYRNAA
LGIDSHWLGNDGIVNTISMNGPKRGSNDRIVPYDGTLKKGVWNDMGTYKVDHLEVI
GVDPNPSFNIRAFYLRLAEQLASLRP
Geobacillus zalihae Lipase:
MKCCRIMFVLLGLWFVFGLSVPGGRTEAASLRANDAPIVLLHGFTGWGREEMFGFK
YWGGVRGDIEQWLNDNGYRTYTLAVGPLSSNWDRACEAYAQLVGGTVDYGAAHA
AKHGHARFGRTYPGLLPELKRGGRIHIIAHSQGGQTARMLVSLLENGSQEEREYAKA
HNVSLSPLFEGGHHFVLSVTTIATPHDGTTLVNMVDFTDRFFDLQKAVLEAAAVASN
VPYTSQVYDFKLDQWGLRRQPGESFDHYFERLKRSPVWTSTDTARYDLSVSGAEKL
NQWVQASPNTYYLSFSTERTYRGALTGNHYPELGMNAFSAVVCAPFLGSYRNPTLG
IDDRWLENDGIVNTVSMNGPKRGSSDRIVPYDGTLKKGVWNDMGTYNVDHLEIIGV
DPNPSFDIRAFYLRLAEQLASLQP
Moraxella sp. (strain TA144) Lipase 2:
MPILPVPALNALLTKTIKTIKTGAAKNAHQHHVLHHTLKGLDNLPAPVLERINRRLK
ASTAEQYPLADAHLRLILAISNKLKRPLAIDKLPKLRQKFGTDAVSLQAPSVWQQNA
DASGSTENAVSWQDKTIANADGGDMTVRCYQKSTQNSERKSTDEAAMLFFHGGGF
CIGDIDTHHEFCHTVCAQTGWAVVSVDYRMAPEYPAPTALKDCLAAYAWLAEHSQ
SLGASPSRIVLSGDSAGGCLAALVAQQVIKPIDALWQDNNQAPAADKKVNDTFKNS
LADLPRPLAQLPLYPVTDYEAEYPSWELYGEGLLLDHNDAEVENSAYTQHSGLPQS
HPLISVMHGDNTQLCPSYIVVAELDILRDEGLAYAELLQKEGVQVQTYTVLGAPHGF
INLMSVHQGLGNQTTYIINEFACLVQNLLTSEGDKPNLRA
Mycobacterium tuberculosis (strain ATCC 25618/H37Rv) Triacylglycerol Lipase:
MVSYVVALPEVMSAAATDVASIGSVVATASQGVAGATTTVLAAAEDEVSAAIAALF
SGHGQDYQALSAQLA VFHERFVQALTGAAKGYAAAELANASLLQSEFASGIGNGFA
TIHQEIQRAPTALAAGFTQVPPFAAAQAGIFTGTPSGAAGFDIASLWPVKPLLSLSALE
THFAIPNNPLLALIASDIPPLSWFLGNSPPPLLNSLLGQTVQYTTYDGMSVVQITPAHP
TGEYVVAIHGGAFILPPSIFHWLNYSVTAYQTGATVQVPIYPLVQEGGTAGTVVPAM
AGLISTQIAQHGVSNVSVVGDSAGGNLALAAAQYMVSQGNPVPSSMVLLSPWLDV
GTWQISQAWAGNLAVNDPLVSPLYGSLNGLPPTYVYSGSLDPLAQQAVVLEHTAVV
QGAPFSFVLAPWQIHDWILLTPWGLLSWPQINQQLGIAA
Pelosinus fermentans DSM 17108 Lipase:
MNSYPIVLVHGFMGWGRNEVLGLKYWGGITDYEQELSSYGYTAYTATVGPVSSNW
DRACELYAYIKGGTVDYGHAHSTQKGHSRYGRTYPGLYPEWGNLTTEGKVNKIHL
VAHSMGGQTVRTLVQLLKEGSEEERNTTPSQLSSLFAGGKSWVHSITTIASPHDGTTL
ADGINIFGDFAKNLVASLASFTGAGEKLIYDFKLDQWGLNRKSGESLTDYTNRVENS
AIWNSTNDLANWDLSTDGARVLNQWVKAQSDIYYFSYSTCATVPSILTSNELPHVIY
MTPLLYPFGRFIGSYTRNEQGRVIIDNSWKPNDGVVNTISQNGPKIWSSDKIVNYNGV
PQIGKWNSMPLLDTIDHMDACGIGTNALTLSWYKGLAEKLSQLTISNLE
Photorhabdus luminescens (Xenorhabdus luminescens) Lipase 1:
MKRSFIFAPGMLALSISAISNAHAYNNLYVFGDSLSDGGNNGRYTVDGINGTESKLY
NDFIAQQLGIELVNSKKGGTNYAAGGATAVADLNNKHNTQDQVMGYLASHSNRAD
HNGMYVHWIGGNDVDAALRNPADAQKIITESAMAASSQVHALLNAGAGLVIVPTV
PDVGMTPKIMEFVLSKGGATSKDLAKIHAVVNGYPTIDKDTRLQVIHGVFKQIGSDV
SGGDAKKAEETTKQLIDGYNELSSNASKLVDNYNQLEDMALSQENGNIVRVDVNAL
LHEVIANPLRYGFLNTIGYACAQGVNAGSCRSKDTGFDASKPFLFADDFHPTPEAHHI
VSQYTVSVLNAPYRVMLLTNANNVPVKGALASLDGRLQQLRNVDNEQGKLGVFGG
YSGNHSHTLTLGSDYQIMDNILLGGMISRYQDNSSPADNFHYDGRGYVFTAYGLWR
YYDKGWISGDLHYLDMKYEDITRGIVLNDWLRKENASTSGHQWGGRITAGWDIPLT
SAVTTSPIIQYAWDKSYVKGYRESGNNSTAMHFGEQRYDSQVGTLGWRLDTNFGYF
NPYAEVRFNHQFGDKRYQIRSAINSTQTSFVSESQKQDTHWREYTIGMNAVITKDW
GAFASISRNDGDVQNHTYSFSLGVNASF
Proteus mirabilis Alpha/beta Hydrolase:
MSTKYPIVLVHGLAGFNEIVGFPYFYGIADALRQDGHQVFTASLSAFNSNEVRGKQL
WQFVQTLLQETQAKKVNFIGHSQGPLACRYVAANYPDSVASVTSINGVNHGSEIAD
LYRRIMRKDSIPEYIVEKVLNAFGTIISTESGHRGDPQDAIAALESLTTEQVTEFNNKY
PQALPKTPGGEGDEIVNGVHYYCFGSYIQGLIAGEKGNLLDPTHAAMRVLNTFFTEK
QNDGLVGRSSMRLGKLIKDDYAQDHIDMVNQVAGLVGYNEDIVAIYTQHAKYLAS
KQL
Pseudomonas aeruginosa (strain ATCC 15692/DSM 22644/CIP 104116/JCM 14847/
LMG 12228/1C/PRS101/Triacylglycerol Lipase:
PAO1)MKKKSLLPLGLAIGLASLAASPLIQASTYTQTKYPIVLAHGMLGFDNILGVDY
WFGIPSALRRDGAQVYVTEVSQLDTSEVRGEQLLQQVEEIVALSGQPKVNLIGHSHG
GPTIRYVAAVRPDLIASATSVGAPHKGSDTADFLRQIPPGSAGEAVLSGLVNSLGALI
SFLSSGSTGTQNSLGSLESLNSEGAARFNAKYPQGIPTSACGEGAYKVNGVSYYSWS
GSSPLTNFLDPSDAFLGASSLTFKNGTANDGLVGTCSSHLGMVIRDNYRMNHLDEV
NQVFGLTSLFETSPVSVYRQHANRLKNASL
Psychrobacter immobilis Lipase 1:
MLLKRLCFAALFSLSMVGCTNAPNALAVNTTQKIIQYERNKSDLEIKSLTLASGDKM
VYAENGNVAGEPLLLIHGFGGNKDNFTRIARQLEGYHLIIPDLLGFGESSKPMSADYR
SEAQRTRLHELLQAKGLASNIHVGGNSMGGAISVAYAAKYPKDVKSLWLVDSAGF
WSAGIPKSLEGATLENNPLLIKSNEDFYKMYDFVMYKPPYLPKSVKAVFAQERIKNK
ELDAKILEQIVTDNVEERAKIIAQYKIPTLVVWGDKDQIIKPETVNLIKKIIPQAQVIM
MEDVGHVPMVEALDETADNYKAFRSILEAQR
Serratia marcescens Extracellular Lipase:
MGIFSYKDLDENASKALFSDALAISTYAYHNIDNGFDEGYHQTGFGLGLPLTLITALI
GSTQSQGGLPGLPWNPDSEQAAQEAVNNAGWSVISATQLGYAGKTDARGTYYGET
AGYTTAQAEVLGKYDSEGNLTAIGISFRGTSGPRESLIGDTIGDVINDLLAGFGPKGY
ADGYTLKAFGNLLGDVAKFAQAHGLSGEDVVVSGHSLGGLAVNSMAAQSDANWG
GFYAQSNYVAFASPTQYEAGGKVINIGYENDPVFRALDGTSLTLPSLGVHDAPHTSA
TNNIVNFNDHYASDAWNLLPFSILNIPTWLSHLPFFYQDGLMRVLNSEFYSLTDKDST
IIVSNLSNVTRGNTWVEDLNRNAETHSGPTFIIGSDGNDLIKGGKGNDYLEGRDGDDI
FRDAGGYNLIAGGKGHNIFDTQQALKNTEVAYDGNTLYLRDAKGGITLADDISTLRS
KETSWLIFNKEVDHQVTAAGLKSDSGLKAYAAATGGDGDDVLQARSHDAWLFGN
AGNDTLIGHAGGNLTFVGGSGDDILKGVGNGNTFLFSGDFGRDQLYGFNASDKLVFI
GTEGASGNIRDYATQQNDDLVLAFGHSQVTLIGVSLDHISTDQVVLA
Staphylococcus aureus Lipase 2:
MLRGQEERKYSIRKYSIGVVSVLAATMFVVSSHEAQASEKTSTNAAAQKETLNQPG
EQGNAITSHQMQSGKQLDDMHKENGKSGTVTEGKDTLQSSKHQSTQNSKTIRTQND
NQVKQDSERQGSKQSHQNNATNNTERQNDQVQNTHHAERNGSQSTTSQSNDVDKS
QPSIPAQKVIPNHDKAAPTSTTPPSNDKTAPKSTKAQDATTDKHPNQQDTHQPAHQII
DAKQDDTVRQSEQKPQVGDLSKHIDGQNSPEKPTDKNTDNKQLIKDALQAPKTRST
TNAAADAKKVRPLKANQVQPLNKYPVVFVHGFLGLVGDNAPALYPNYWGGNKFK
VIEELRKQGYNVHQASVSAFGSNYDRAVELYYYIKGGRVDYGAAHAAKYGHERYG
KTYKGIMPNWEPGKKVHLVGHSMGGQTIRLMEEFLRNGNKEEIAYHKAHGGEISPL
FTGGHNNMVASITTLATPHNGSQAADKFGNTEAVRKIMFALNRFMGNKYSNIDLGL
TQWGFKQLPNESYIDYIKRVSKSKIWTSDDNAAYDLTLDGSAKLNNMTSMNPNITY
TTYTGVSSHTGPLGYENPDLGTFFLMATTSRIIGHDAREEWRKNDGVVPVISSLHPSN
QPFVNVTNDEPATRRGIWQVKPIIQGWDHVDFIGVDFLDFKRKGAELANFYTGIIND
LLRVEATESKGTQLKAS
Streptomyces coelicolor (strain ATCC BAA-471/A3(2)/M145) Lipase 2:
MPKPALRRVMTATVAAVGTLALGLTDATAHAAPAQATPTLDYVALGDSYSAGSGV
LPVDPANLLCLRSTANYPHVIADTTGARLTDVTCGAAQTADFTRAQYPGVAPQLDA
LGTGTDLVTLTIGGNDNSTFINAITACGTAGVLSGGKGSPCKDRHGTSFDDEIEANTY
PALKEALLGVRARAPHARVAALGYPWITPATADPSCFLKLPLAAGDVPYLRAIQAHL
NDAVRRAAEETGATYVDFSGVSDGHDACEAPGTRWIEPLLFGHSLVPVHPNALGER
RMAEHTMDVLGLD
Vibrio cholerae serotype 01 (strain ATCC 39315/E1 Tor Inaba N16961) )
Triacylglycerol Lipase:
MNKIIILIALSLFSSSIWAGTSAHALSQQGYTQTRYPIVLVHGLFGFDTLAGMDYFHGI
PQSLTRDGAQVYVAQVSATNSSERRGEQLLAQVESLLAVTGAKKVNLIGHSHGGPTI
RYVASVRPDLVASVTSIGGVHKGSAVADLVRGVIPSGSVSEQVAVGLTQGLVALIDL
LSGGKAHPQDPLASLAALTTEGSLKFNQYYPEGVPTSACGEGAYQVNGVRYYSWSG
AATVTNILDPSDVAMGLIGLVFNEPNDGLVATCSTHLGKVIRDDYRMNHLDEINGLL
GIHSLFETDPVTLYRQHANRLKQAGL
Cupriavidus metallidurans (strain ATCC 43123/DSM 2839/NBRC 102507/CH34)
(Ralstonia metallidurans)) Triacylglycerol Lipase:
MEHGNRRGPGRACAHQDTVTLKAGTRRSTSAIARTAAGTAAAIAAAIAMAWPASA
TASSDYAKTRYPIVLVHGLTGAAKMGGVIDYWYGIPEVLRANGATVYVSTVPSENS
DEERALALQAYVRAVKLETGADKVNLIGHSQGGPTSRMLAAMSPQDVASVTTIGSP
HRGSQLADVVLDVINGISSIPLAGPALVNVIQGVTNLLGWFNGLENGQALNQDAIAS
ARTLTSQGATEQNARLNATLAPGSQSALGPDCDTPGAVAEQRQGRDASGKLVTYTQ
AAYSWTGRGGPINLLRSNLLDPSTAAMAAAAGVMGMKGAGPNDGMVSVCSAKWG
RVLSTDYYWNHLDEINQMLGMYQDVDPRTVILNHANRLRNDQL
Cupriavidus necator (strain ATCC 17699/DSM 428/KCTC 22496/NCIMB 10442/
H16/Stanier 337) (Ralstonia eutropha) Triacylglycerol Lipase:
MEHSGHHACGRRTPALREQKAGKALRRLAGTATLVAAAMLAQPAPALAASGDYA
KTRYPIVLVHGLTGAARMGGVLDYWYGIPEVLRANGAQVYVATVPSFNSDEERAL
ALQAYVRAVKLESGADKVNLIGHSQGGPTSRMLAAMSPQDVASVTTIGSPHRGSEV
ADTVLDLINGVNSIPIAGPVLVGIIQGVLDTVGWFNGIGNGQALDQDALASLKILTTR
GAAEQNARLDATLVPGTKSALGPDCNTAGAVSEQRQARDAAGNFVTHTQAAYSWT
GQGGPFSLLRSNLLDPTTVMMSASAGLMDLKGAGPNDGLVSVCSSKWGRVLATGY
YWNHLDEVNQMAGLYQDVDPRTVILSHANRLRNDQL
Cupriavidus taiwanensis (strain DSM 17343/BCRC 17206/CCUG 44338/CIP
107171/LMG 19424/R1) (Ralstonia taiwanensis (strain LMG 19424)) Triacylglycerol
Lipase Putative:
MEHGENHACGPRTPVSRPQQSPNPLHRLAGAAAALAAAAMLAQPVPAAAALTANG
DYAKTRYPIVLVHGLTGAAKMAGVLDYWYGIPEVLRDHGAQVYVATVPSFNSDAE
RALALQAYVRAVKLETGADKVNLIGHSQGGPTARVLAAMSPQDVASVTTIGSPHRG
SEVADTVLDLINGIGAIPIAGPVLVSLIQGVFDTVGWFNGISNGQALDQDALASLESLT
TRGAAGQNARLDATLVPGTKSALGPDCDSPGAVSEQRQARDAAGNLVTHRQAAYS
WTGQGGPLSLLRSNLLDPSTVMMSTSGRLMALKGAGANDGLVSVCSSKWGRVLAT
GYYWNHLDEVNQMAGLYQDADPRTVILNHANRLRNDQL
Clostridium butyricum Lipase:
MNDKRIYFRIRNIIIFTLLMNMIFIFNIMKTKILCYIIGIIVLLIYLYINIIPLRLKKLNKRV
CIMVSGYELIADSSLAVLIEIILYIFLLNTDNLTINSKEFIINGITAVCVLIIPVINGFMRM
MFTSRNLGIVNRLLIVLLWWAPILNFIILNGCCKKVRYEYFYEQSKELRNDVRKENE
VCRTRYPIVMVHGIFFRDWMFINYWGRIPKELMRNGAQIFYGKQQSSNAVCKSAQE
LKENILKIINDTGCEKVNIIAHSKGGLDSRYAISCLGLSKYVASLTTVNTPHRGCKYV
DFLLDKAPDKFKNYVAKKYNRTFVKLGDKNPDFLAGVADLTLEKCSQFNIKVKDVE
GVLYQSVTSKMKNMFSSGFPLNIGYILAKIFDGENDGLVEVSSAKWGNFLGTLTAGK
KGISHGDMVDLTRQDIRGYDVCEFYVDLVRKLKEKGN
Candida albicans (strain SC5314/ATCC MYA-2876) (Yeast) Lipase 4:
MLFLLFLLVAPIYAGLILPTKPSNDPFYNAPAGFEKAAVGEILQSRKTPKPITGVFVPV
KIQNSWQLLVRSEDSFGNPNVIVTTVMEPFNADPSKLASYQVFEDSAKADCAPSYAL
QFGSDVTTIATQVETYLLAPLLDQGYYVVSPDYEGPKSTFTVGKQSGQAVLNSIRAA
LKSGKITNLAEDAKVVMWGYSGGSLASGWAAALQPNYAPELGGNLLGAALGGFVT
NITATAEATDGTVFAGIMANALGGVANEYPEFKQILQNDTDKQSVFDQFDNHCLAD
GVINYIGKHFLSGTNKIFKSGWNILKNPTISKIVEDNGLVYQKQLVPKIPILIYHGAIDQ
IVPIVNVKKTYQNWCDAGIASLEFSEDATNGHITETIVGAPVALTWIINRFNGKQTVS
GCQHVKRTSNFEYPNIPPSILNYFKAALNILIQKGLGPDIQKDQVNPDGLKKIPILV
Aspergillus niger (strain ATCC MYA-4892/CBS 513.88/FGSC A1513) Putative
Lipase atg 15:
MKGLRGHNKKSFWGNTRLSDLLWPVTLLPGLISAYQPVYLGSRQSSPFLPPQIPLGDS
PPLSDTHEFTLRHIFHRGTYQDPDLHRRLDITPDTRLRAVSDAGIESDVVTPNTPLVAS
SQPLIIERLADRRLSVIEEHLVTARSSGSAVALSPSDWVMDTLPGPNVTEKKTVVALA
MMTANDYIEVPGTGDWQDIHGRFNHSGSFGWQGDGLRGHVYADKINSTVVISLKG
TSPALFDGEGTTTNDKINDNLFFSCCCGQGGSYLWRQVCDCQTTLYNANLTCIVEA
MLDENRYYRASLDLYSNITEMYPNANVWLTGHSLGGAVSSLLGLTFGVPVVTFEAV
PEALPAARLGLPSPPGHDSRYPQSRRNTGSYHFGHTADPVYMGTCNGVSSVCTWGG
YAMESACHTGQMCVYDTVEDKGWRVGLSKHRINYVIANVLAGYDDVPPCAPEEEC
YDCELWKFFRSNGSEITTTTSATSTSTSTTTSRTSTCKTPGWWGCLDQTTTTETTSTST
STSTCKTPGWFGCKDPTTTSDITSVPTITTTEPPMTSTTTCKTPGWFGCKDETTTQVIA
PAATLTITEPPVTSTTTGKHLGRF
Diutina rugosa (Yeast) (Candida rugosa) Lipase 1:
MELALALSLIASVAAAPTATLANGDTITGLNAIINEAFLGIPFAEPPVGNLRFKDPVPY
SGSLDGQKFTSYGPSCMQQNPEGTYEENLPKAALDLVMQSKVFEAVSPSSEDCLTIN
VVRPPGTKAGANLPVMLWIFGGGFEVGGTSTFPPAQMITKSIAMGKPIIHVSVNYRV
SSWGFLAGDEIKAEGSANAGLKDQRLGMQWVADNIAAFGGDPTKVTIFGESAGSMS
VMCHILWNDGDNTYKGKPLFRAGIMQSGAMVPSDAVDGIYGNEIFDLLASNAGCGS
ASDKLACLRGVSSDTLEDATNNTPGFLAYSSLRLSYLPRPDGVNITDDMYALVREGK
YANIPVIIGDQNDEGTFFGTSSLNVTTDAQAREYFKQSFVHASDAEIDTLMTAYPGDI
TQGSPFDTGILNALTPQFKRISAVLGDLGFTLARRYFLNHYTGGTKYSFLSKQLSGLP
VLGTFHSNDIVFQDYLLGSGSLIYNNAFIAFATDLDPNTAGLLVKWPEYTSSSQSGNN
LMMINALGLYTGKDNFRTAGYDALFSNPPSFFV
Pseudozyma antarctica (Yeast) (Candida antarctica) Lipase
BMKLLSLTGVAGVLATCVAATPLVKRLPSGSDPAFSQPKSVLDAGLTCQGASPSSVS
KPILLVPGTGTTGPQSFDSNWIPLSTQLGYTPCWISPPPFMLNDTQVNTEYMVNAITA
LYAGSGNNKLPVLTWSQGGLVAQWGLTFFPSIRSKVDRLMAFAPDYKGTVLAGPLD
ALAVSAPSVWQQTTGSALTTALRNAGGLTQIVPTTNLYSATDEIVQPQVSNSPLDSS
YLFNGKNVQAQAVCGPLFVIDHAGSLTSQFSYVVGRSALRSTTGQARSADYGITDCN
PLPANDLTPEQKVAAAALLAPAAAAIVAGPKQNCEPDLMPYARPFAVGKRTCSGIVT
P
Saccharomyces cerevisiae (strain ATCC 204508/S288c) (Baker's yeast)
Triacylglycerol Lipase 3:
MKETAQEYKVSAVIPTLLKNWILRVVYATLDHIPPFVWEILHVITDIYFFWVQKLINY
VRPHSRVIYYNAIKKLDECDTYQMWCQQASVVDEITGANLWRRNFFSRRYDENSVI
EQYSILENMLREEKYDVVKEKFSTTGPCMLRNFAGIGDKKLFTKSLMGTKLLIEQYL
TRILEGLDILNNQTLTPTSFFQRCKLSLGTTALILQGGSLFGLFHLGVIRGLLLQDLMP
NIISGSSMGACVASLFGCLSNEQLKQLLTDDNLLNIIKNDVDLLKSCGYGNLEQHLNL
GTLIQNLIHHGYSQDVYLFIRFVMKYIVKEKTFEEVYQITGKVFNIVIHPTDKSCPNLL
NYVTTPNVLIKSAIECSLGSGVISEDTSLLCKNLENEIEPFLNINKNKQVKFLTPENAN
NPSITESPYTRLTELFNVNNFIVSLARPYLAPLVVNDLKHEIKTSKYYYYKHYPNMPPI
NANTVRKTQRSSSQSPIKAGTVEDLEPEPLMSPVPPSSAVNDSAEYIIPELGIPQLNFTE
MEPLAFKFKYHLERKLKNIATMEFRHRMEVLDNLGLLCSLIKRLIIDEKTPRSATEIA
VVPRMKSLSLTRIIEGQLNNIPYWIKSGERSTWPALALIKTRCAVEFKLDDIIRARRSR
Yarrowia lipolytica (strain CLIB 122/E 150) (Yeast) (Candida lipolytica) Lipase:
MSVTSTSLNGTFNGISEDGIEIFKGIKYANIPYRWAYAERIDDYDNGVEDCTQEGMAC
PQVLPFDYNIEKGPKEMPFDEFECSNLMITRPQGATNLPVFVWIHGGGNLAGNGYCS
DHNPVPFVKHSIVAGRPVLHVMIEYRLSAFGYLAVPDTNGNWVGNWGARDQYTAL
QWISKHIVEFGGDPSQITIGGESAGSIGLHALMVHESMKPKEECIIHNVILSSGTMDRM
GTGTISENAFKPIYDGIKTLVGDINTCSADELLEAQIKAGLDLGFYLQDDFFPPDWRN
VRFKVSRVLLSDVIVDGTNFKNKINPAVRVTPENDFDHKVFKLYNISTEDTWEDYHY
KMMLFKGDETFIRGNQQLELLFEQENIPVWRQLFDQIHPNDPSRLCHHAVDLYYMW
DNWEMPEDKHAVARQYQDTLTKFVYGQDPWPVDKLHYVHDNQFEILDKSQFGDF
RNVPALKFLLGFSAEELGELTKKYTGEGHYTL

Suitable exogenous or heterologous nucleic acid sequences encoding a protein with lipase activity may also include nucleic acid sequences which can be derived as a result of the degenerate genetic code from the amino acid sequences identified above as well as functional equivalents with lipase activity.

For the purposes of the present disclosure, heterologous lipases and the transgenes that express them can be derived from a variety of organisms that natively express such enzymes, such as certain bacteria, cyanobacteria, fungi, algae, and other microbes or animals.

In certain aspects, the C. necator may be engineered to express one or more functional fragments of a lipase. That is, the functional fragment is a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length polypeptide. The functional fragment can generally, but not always, be comprised of a continuous region of the protein, wherein the region has functioning lipase activity.

An engineered strain of C. necator can express a lipase alone or in combination with one or more of digestive enzyme, including but not limited to, other lipase enzymes, a protease, a carbohydrase, and/or a phytase.

### E. Phytase Enzymes

C. necator does not naturally produce significant amounts of digestive enzymes exhibiting phytase activity sufficient to catalyze phytic acid. Accordingly, the present disclosure provides for the introduction of nucleic acids encoding a phytase enzyme. In the context of a single cell protein product, for example, in the use of animal feed produces, may have a significant beneficial effect on the performance of an animal, including improving one or more of the following: feed conversion ratio (FCR), ability to digest a raw material (e.g. nutrient digestibility, such as amino acid digestibility), nitrogen retention, survival, carcass yield, growth rate, weight gain, feed efficiency animals resistance to necrotic enteritis, immune response of the subject, the growth of beneficial bacteria in the gastrointestinal tract of a subject.

Thus, the engineered C. necator of the present disclosure may be modified to express a phytase enzyme.

Suitable exogenous or heterologous nucleic acid sequences that encode a protein with phytase activity include, but are not limited to, a nucleic acid sequences that code for proteins or polypeptides having phytase activity with at least 50% identity or similarity (e.g., at least 60%, at least 70%, at least 80%, at least 90%, at least 95%, at least 96%, at least 97%, at least 98%, or at least 99%) at the amino acid level with the following sequences:

Bacillus sp. DS11 3-phytase:
MNHSKTLLLTAAAGLMLTCGAVSSQAKHKLSDPYHFTVNAAAETEPVDTAGDAAD
DPAIWLDPKNPQNSKLITTNKKSGLAVYSLEGKMLHSYHTGKLNNVDIRYDFPLNG
KKVDIAAASNRSEGKNTIEIYAIDGKNGTLQSITDPNRPIASAIDEVYGFSLYHSQKTG
KYYAMVTGKEGEFEQYELNADKNGYISGKKVRAFKMNSQTEGMAADDEYGSLYIA
EEDEAIWKFSAEPDGGSNGTVIDRADGRHLTPDIEGLTIYYAADGKGYLLASSQGNS
SYAIYERQGQNKYVADFQITDGPETDGTSDTDGIDVLGFGLGPEYPFGLFVAQDGENI
DHGQKANQNFKMVPWERIADKIGFHPQVNKQVDPRKMTDRSGK
Bacillus licheniformis β propeller phytase:
MNFYKTLALSTLAASLLSPSWSILPRAEASAYKDFSVTADAETEPVDTPDDAADDPAI
WVHPKQPEKSRLITTNKKSGLIVYDLNGKQLAAYPFGKLNNVDLRYNFPLDGKKIDI
AGASNRSDGKNTVEIYAFDGEKNKLKNIVNPQKPIQTDIEEVYGFSLYHSQKTGKFY
AMVTGKNVEFEQYELFDNGKGQVEGKKVRSFKMSSQTEGLAADDEYGKMYIAEED
AAIWSFSAEPNGGDKGKIVDRAGGTHLTADIEGLTIYYGEDGEGYLIASSQGDNRYAI
YDRRGKNDYVTDFSIDDGKEIDGTSDTDGIDVIGFGLGKKYPYGIFVAQDGENTENG
QPANQNFKIVSWEKIADALDDKPDIDDQVNPRKLKKRAK
Citrobacter braakii phytase:
MSTFIIRLLIFSLLCGSFSIHAEEQNGMKLERVVIVSRHGVRAPTKFTPIMKDVTPDQW
PQWDVPLGWLTPRGGELVSELGQYQRLWFTSKGLLNNQTCPSPGQVAVIADTDQRT
RKTGEAFLAGLAPKCQIQVHYQKDEEKNDPLFNPVKMGKCSFNTLKVKNAILERAG
GNIELYTQRYQSSFRTLENVLNFSQSETCKTTEKSTKCTLPEALPSEFKVTPDNVSLPG
AWSLSSTLTEIFLLQEAQGMPQVAWGRITGEKEWRDLLSLHNAQFDLLQRTPEVARS
RATPLLDMIDTALLTNGTTENRYGIKLPVSLLFIAGHDTNLANLSGALDLKWSLPGQP
DNTPPGGELVFEKWKRTSDNTDWVQVSFVYQTLRDMRDIQPLSLEKPAGKVDLKLI
ACEEKNSQGMCSLKSFSRLIKEIRVPECAVTE
Escherichia coli phytase:
MKAILIPFLSLLIPLTPQSAFAQSEPELKLESVVIVSRHGVRAPTKATQLMQDVTPDA
WPTWPVKLGWLTPRGGELIAYLGHYQRQRLVADGLLAKKGCPQSGQVAIIADVDE
RTRKTGEAFAAGLAPDCAITVHTQADTSSPDPLFNPLKTGVCQLDNANVTDAILSRA
GGSIADFTGHRQTAFRELERVLNFPQSNLCLKREKQDESCSLTQALPSELKVSADNVS
LTGAVSLASMLTEIFLLQQAQGMPEPGWGRITDSHQWNTLLSLHNAQFYLLQRTPEV
ARSRATPLLDLIKTALTPHPPQKQAYGVTLPTSVLFIAGHDTNLANLGGALELNWTL
PGQPDNTPPGGELVFERWRRLSDNSQWIQVSLVFQTLQQMRDKTPLSLNTPPGEVKL
TLAGCEERNAQGMCSLAGFTQIVNEARIPACSL

Suitable exogenous or heterologous nucleic acid sequences encoding a protein with phytase activity may also include nucleic acid sequences which can be derived as a result of the degenerate genetic code from the amino acid sequences identified above as well as functional equivalents with phytase activity.

For the purposes of the present disclosure, heterologous phytases and the transgenes that express them can be derived from a variety of organisms that natively express such enzymes, such as certain bacteria, cyanobacteria, fungi, algae, and other microbes or animals.

In certain aspects, the C. necator may be engineered to express one or more functional fragments of a phytase. That is, the functional fragment is a polypeptide fragment of a protein that has at least 25%, e.g., at least 30%, at least 40%, at least 50%, at least 60%, at least 70%, at least 75%, at least 80%, at least 85%, at least 90%, at least 95%, at least 98%, at least 99%, or at least 100% of the activity of the corresponding mature, full-length polypeptide. The functional fragment can generally, but not always, be comprised of a continuous region of the protein, wherein the region has functioning phytase activity.

An engineered strain of C. necator can express a phytase alone or in combination with one or more of digestive enzyme, including but not limited to, other phytase enzymes, a protease, a lipase, and/or a carbohydrase.

### F. Expression of Transgenes

The nucleic acid sequences (e.g., transgenes) that are transfected or otherwise incorporated into the engineered C. necator of the present disclosure can be incorporated into an expression cassette. In doing so, the nucleic acid sequences that encode a protease, carbohydrase, lipase, and/or phytase can be operably linked with one or more regulatory signals for initiating, inducing, or enhancing gene expression. These regulatory sequences are intended to make possible the specific expression of the genes and proteins and may be referred to as a “gene construct.” The gene construct may also comprise one or more enhancer sequences in operable linkage with the promoter, which make possible an enhanced expression of the nucleic acid sequence. Additional advantageous sequences, such as further regulatory elements or terminator sequences, may also be inserted at the 3′ end of the DNA sequences. The transgenes may be present in one or more copies of the expression cassette (or gene construct).

The regulatory sequences or factors preferably have a positive influence on the gene expression of the introduced genes and thereby increase expression. The regulatory elements can advantageously be strengthened at the transcription level by using strong transcription signals such as promoters and/or enhancers.

For the purposes of the present disclosure, an expression cassette or expression vector may comprise one transgene encoding a protease, carbohydrase, lipase, or phytase or other enzyme. Alternatively, the expression cassette or expression vector may comprise multiple transgenes encoding a combination of proteases, carbohydrases, lipases, or phytases, or other enzymes.

Various methods can be used to introduce an expression vector into the C. necator.

Such methods are generally described in Sambrook et ah, Molecular Cloning: A Laboratory Manual, Cold Springs Harbor Laboratory, New York (1989, 1992), in Ausubel et ah, Current Protocols in Molecular Biology, John Wiley and Sons, Baltimore, Md. (1989), Chang et ah, Somatic Gene Therapy, CRC Press, Ann Arbor, Mich. (1995), Vega et ah, Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, and infection with recombinant viral vectors. Stable expression of transgenes can also be achieved, for example, using CRISPR-based systems, TALON, or zinc finger nucleases.

### VII. Single Cell Protein and Uses Thereof

The present disclosure provides edible products, food, and others products, such as single cell protein products, that include protein derived from the engineered C. necator bacterium provided herein. Additionally provided are methods for producing amino acids, digestive enzymes proteins, and other biological nutrients using the engineered C. necator described herein.

Current single cell protein (SCP) products are largely lacking in desirable digestive enzymes. The engineered C. necator disclosed herein can be utilized in methods of preparing a SCP product that includes desirable digestive enzymes and does not require further fortification. The methods may comprise culturing an engineered strain of C. necator, thereby creating biomass comprising protein and digestive enzymes. The methods may also comprise isolating the C. necator or the biomass, and preparing a single-cell protein product from the C. necator or the biomass. The protein-rich biomass may be used as a single cell protein (SCP) product. The biomass may be utilized as an edible product, for instance, an animal feed or human food.

In some embodiments, the animal feed is a fish feed, a livestock or ruminant feed, a swine feed, goat feed, llama feed, turkey feed, a poultry feed, a rodent feed, a dog feed, and a cat feed. The animal feed may be a livestock feed or a domesticated animal feed. In yet other aspects, the human food is a yogurt, a smoothie, a bread product, a pasta product, a nutritional bar, a chip or cracker, a plant-based meat substitute, a cheese, a plant-based cheese, and a powdered nutritional supplement.

One of the major challenges in utilizing biosystems for food production is obtaining the proper dietary balance between the quantities of protein, carbohydrate, and fat. The microbial systems generally considered for food synthesis tend to produce biomass disproportionately high in protein and digestive enzymes. The engineered C. necator strains of the present disclosure can be used to produce a SCP product with a higher proportion of digestive enzymes, particularly, proteases, carbohydrases, lipases, and/or phytases. Thus, the present disclosure provides food products, including human foods and animal feeds comprising a SCP produced from or including the engineered C. necator described herein.

A SCP product, “protein product,” or “microbial protein product” (e.g., one or more of single cell protein, cell lysate, protein concentrate, protein isolate, protein extract, protein hydrolysate, free amino acids, peptides, oligopeptides, or combinations thereof), derived from one or more engineered C. necator strains described herein, may be processed or incorporated into an edible food composition for human and/or animal consumption, a cosmetic, a pharmaceutical product, or a fertilizer.

A food composition (i.e., food product) may be, for example, a food item, a food ingredient, a nutritional product, an animal feed, and/or a pet food product. In some embodiments, the food composition may contain any of at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A cosmetic or pharmaceutical composition may contain any of at least about 0.01%, at least about 0.05%, at least about 0.1%, at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

A fertilizer may contain any of at least about 0.5%, at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to 100% microbial protein product by weight, e.g., by weight on a dry weight basis.

The biomass that is produced from culturing the engineered C. necator strains disclosed herein results in a desirable protein and digestive content. The biomass can have a protein content higher than or at least about any of about 40%, about 50%, about 60%, about 70%, about 80%, about 90%, or about 95% by weight, and a fat content of about 60%, about 50%, about 40%, about 30%, about 20%, about 15%, about 10%, or about 5% by weight.

The biomass or an SCP product created therefrom may comprise free amino acids, polypeptides, proteins, and/or digestive enzymes. In some embodiments, an SCP product derived from the engineered C. necator strains disclosed herein comprises an increased digestive enzyme content relative to an SCP product derived from a wild type or naturally occurring strain of C. necator. For instance, the SCP product comprises about 0.25 wt % to about 25 wt %, about 0.5 wt % to about 25 wt %, about 1 wt % to about 25 wt %, about 5 wt % to about 25 wt %, about 10 wt % to about 25 wt %, about 15 wt % to about 25 wt %, about 20 wt % to about 25 wt %, about 0.5 wt % to about 20 wt %, about 1 wt % to about 20 wt %, about 5 wt % to about 20 wt %, about 10 wt % to about 20 wt %, about 15 wt % to about 20 wt %, about 0.5 wt % to about 15 wt %, about 1 wt % to about 15 wt %, about 5 wt % to about 15 wt %, about 10 wt % to about 15 wt %, about 0.5 wt % to about 10 wt %, about 1 wt % to about 10 wt %, about 5 wt % to about 10 wt %, about 0.5 wt % to about 5 wt %, or about 1 wt % to about 5 wt % of digestive enzymes.

Compositions comprising the disclosed SCP product can find application in various industries such as food, pharmaceutical, nutraceutical, cosmetic, agriculture, consumer goods, construction etc. The compositions can be used as food agents for human or animal consumption, cosmetic agents, pharmaceutical agents, nutritional agents, fertilizer and other agricultural agents, industrial agents, or any combination thereof. Illustrative applications include animal feed, food and beverages, infant formula, toddler formula, special dietary needs formula, reduced allergenicity formulas, skin-care and hair-care compositions, fertilizers, etc.

In some aspects, the SCP products as described herein are utilized in the production of a vegetarian or vegan food product. In certain embodiments, the SCP products are utilized in the production of an organic food product and/or pesticide-free and/or herbicide-free and/or fungicide-free and/or antibiotic-free food product. The SCP products may be utilized in a probiotic food product or in a prebiotic food product. In some aspects the SCP product may not include animal protein or fats.

In some embodiments, the SCP product can be incorporated into food products including, but not limited to, dairy products, dairy replacement products, meat products (including livestock, game, poultry, fish, or seafood products), meat replacement and/or imitation meat products (including imitation livestock, game, poultry, fish, or seafood products), bakery products, confections, health and protein bars, protein powders, sports and/or energy drinks, and/or protein shakes and/or smoothies. The type of food and beverage is not particularly limited, and can include, for example, noodles, instant noodles, soups, instant soups, pasta, microwave foods, canned foods, freeze-dried foods, soft drinks, fruit juice drinks, vegetable drinks, infant formula, toddler formula, non-dairy milk, coffee drinks, tea drinks, nutritional beverages, powdered beverages, protein powders, nutritional supplements, concentrated beverages, alcoholic beverages, breads, cake mixes, rice cakes, flour products, chewing gum, gummies, chocolate, caramel, cookies, snacks, chips, pretzels, crackers, biscuits, cakes, pies, confectionery, sauces, processed seasonings, flavor seasonings, cooking mixes, curries, stews, sauces, dressings, oils and fats, butter, margarine, mayonnaise and other condiments, milk drinks, yogurt, lactic acid bacteria drinks, ice creams, cream processed fish products, processed livestock products, agricultural canned products, jams and marmalades, pickles, cereals, nutritional foods, vegan or vegetarian meat substitutes, and the like. In certain embodiments, protein products are textured for incorporation into meat products and/or imitation meat products.

In some aspects, the SCP product has a high ratio of digestive enzymes. In certain embodiments, the SCP product may have an amino acid content is substantially similar, very close, or identical to that recommended by the United Nations Food and Agriculture Organization as “ideal.” In certain embodiments, food products made using the SCP products of the present disclosure represent healthy and/or low-calorie foods. The SCP product may be formed into fibers and/or thermally extruded and/or coagulated into a gel. Gel coagulation occurs at pH falling in a range of about 3 to about 6 upon heating. In some aspects, one or more properties of the SCP product makes it well suited for incorporation into food products, including but not limited to dairy products, dairy replacement products, meat products, meat replacement and/or imitation meat products, bakery products, confections, health and protein bars, protein powders, sports and/or energy drinks, and/or protein shakes and/or smoothies. The SCP product may be used as a meat extender, for example, as a meat extender in food products. In some aspects, water absorption and/or fat binding properties of the SCP product aids in reducing shrinkage (fat and water loss) on cooking and/or enhances the moisture and texture of the cooked patty or other meat or food item.

The SCP product may impart improved nutrition, water absorption, fat binding properties, texture, and/or eating qualities to a food product, such as a cereal based product. The SCP product may be used to fortify or is otherwise incorporated into a cereal based product. The cereal based product may be a breakfast cereal, cookie, cake, pie, brownie, muffin, or bread. In some aspects, the protein product may be used as a replacement for milk proteins (e.g. sodium caseinate) and/or as a vitamin and/or mineral supplement in milk or dairy products. The protein SCP ingredient may be used in one or more of non-fat dried milk, powdered milk, or dairy type drinks, such as, but not limited to, instant breakfast mixes, or imitation dairy type drinks including but not limited to soy milk, rice milk, and almond milk. The SCP product ingredient may be used in nutritionally fortified (e.g., protein, vitamin, and/or mineral fortified) candies, deserts, or treats.

The SCP product may be processed to produce a food product or ingredient thereof, in a process that includes heating the protein product, optionally in combination with other ingredients, optionally under shearing agitation, followed by extrusion to produce a product of desired texture (e.g., chewy, crunchy, crispy, resists dispersion in water, etc.). The SCP product can be processed to produce a food product or ingredient thereof, in a process that includes combining the SCP product with one or more additional protein sources (including, but not limited to, pea, rice, glutinous rice, wheat, gluten, soy, hemp, canola, insects, algae, and/or buckwheat).

In some aspects, free amino acids are included, either as part of the SCP product or supplemental to the SCP product, to impart a desired flavor. In one non-limiting embodiment, glutamic acid is included, thereby imparting an umami flavor to the food product.

In some aspects, for example, in a meat substitute or artificial meat product, a hydrogel, lipogel, and/or emulsion can be combined the SCP product, for example, as an agent release system (e.g., for release of a coloring agent, a flavor agent, a fatty acid, a leavening agent, a gelling agent (e.g., bicarbonate (e.g., potassium bicarbonate), calcium hydroxide, and/or alginate (e.g., sodium or potassium alginate)), wherein the agent(s) may be released during cooking of the food product to simulate animal meat).

In some aspects, a food product comprising the SCP produce may also include one or more plant protein source such as, but not limited to, pea, rice, glutinous rice, wheat, gluten, soy, hemp, canola, insects, algae, and/or buckwheat, in combination with a protein product produced by microorganisms as described herein (e.g., one or more of single cell protein, cell lysate, protein concentrate, protein isolate, protein extract, protein hydrolysate, free amino acids, peptides, oligopeptides, or combinations thereof), wherein the protein product imparts a flavor to the food composition, such as, for example, a meat-like flavor (including a livestock, game, poultry, or seafood meat-like flavor).

In some aspects, a food product, for example, a meat substitute or artificial meat product, includes a heme compound, such as a heme-containing polypeptide. In one embodiment, the food product includes heme (e.g., heme-containing polypeptide) from the microorganism from which the protein product is derived.

A meat substitute or artificial or imitation meat product (e.g., a livestock (e.g., beef, pork), game, poultry, fish, or seafood analogue product) may include a protein product produced by microorganisms as described herein (e.g., one or more of single cell protein, cell lysate, protein concentrate, protein isolate, protein extract, protein hydrolysate, free amino acids, peptides, oligopeptides, or combinations thereof). In some embodiments, the meat analogue product is a vegan product that does not contain any ingredients from animal sources. In some embodiments, an enhanced meat product which contains animal protein (e.g., a beef, poultry, pork, fish, seafood, or egg product) and comprises a protein product ingredient produced by microorganisms as described herein (e.g., one or more of single cell protein, cell lysate, protein concentrate, protein isolate, protein extract, protein hydrolysate, free amino acids, peptides, oligopeptides, or combinations thereof)), is provided. For example, the protein product may be included as an extender in an enhanced meat product or in a meat analogue product, e.g., the SCP product replaces any of at least about 10%, at least about 20%, at least about 30%, at least about 40%, at least about 50%, at least about 60%, or at least about 70% of the meat ingredient or an artificial or imitation meat ingredient (for example, a plant-based artificial or imitation meat analogue ingredient) to produce the enhanced meat product or meat analogue/imitation meat product, respectively.

In some aspects, the protein product is used as an aquaculture feed or in an aquaculture feed formulation. In some embodiments, the protein-rich biomass is used as a high-protein substitute for fishmeal used in aquaculture and/or other animal feed products. The animal feed may include up to 20% (w/w) or up to 10% (w/w) SCP product, wherein the SCP product comprises engineered C. necator cells described herein.

Protein and/or biomass produced according to the present disclosure may be converted to animal feed using methods and processes well known in the art and science of chemistry, chemical engineering, and food science. The feed produced through the disclosure may be used to grow organisms including but not limited to one or more of the following: other microorganisms, yeast, fungi, zooplankton, shellfish (e.g., shrimp, prawns, crabs, scallops, clams, mussels, etc.) or other invertebrates, fish, birds, and mammals. In certain non-limiting embodiments, the fish include but are not limited to one or more of: tilapia, tuna, salmon, cod, cobia, and haddock. The birds may include, but are not limited, to chickens, pheasants, or turkeys. The mammals may include but are not limited to one or more of: rodents, rabbits, goats, sheep, pigs, cows, horses, deer, dogs, cats, buffalo, llamas, alpacas, non-human primates, and aquatic mammals (e.g., dolphins, whales, manatees, etc.). The feed may be used to grow live-feed that in turn sustain finfish larvae through the first weeks of life. The feed produced may be used to grow zooplankton organisms including but not limited to one or more of the following: rotifers [Phylum Rotifera]; order Cladoceran (e.g., Daphnia sp., Moina sp.); sub-class Copepoda (e.g., Cyclops); Brine shrimp (Anemia sp.).

In some embodiments, the animal feed comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the animal feed comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the animal feed comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

The microbial cells of the present disclosure may be boiled prior to feeding to another organism (e.g., human or animal). The cells may sonicated, or otherwise lysed or ruptured prior to feeding to another organism (e.g., human or animal).

In some embodiments, the fertilizer comprises up to 1% (w/w), up to 5% (w/w), up to 10% (w/w), up to 15% (w/w), up to 20% (w/w), up to 25% (w/w), up to 30% (w/w), up to 35% (w/w), up to 40% (w/w), up to 45% (w/w), up to 50% (w/w), up to 55% (w/w), up to 60% (w/w), up to 65% (w/w), up to 70% (w/w), up to 75% (w/w) or more of one or more of the disclosed SCP products. In some embodiments, the fertilizer comprises at least 1% (w/w), at least 5% (w/w), at least 10% (w/w), at least 15% (w/w), at least 20% (w/w), at least 25% (w/w), at least 30% (w/w), at least 35% (w/w), at least 40% (w/w), at least 45% (w/w), at least 50% (w/w), at least 55% (w/w), at least 60% (w/w), at least 65% (w/w), at least 70% (w/w), at least 75% (w/w) or more of one or more of the disclosed SCP products. In some embodiments, the fertilizer comprises about 1% (w/w), about 5% (w/w), about 10% (w/w), about 15% (w/w), about 20% (w/w), about 25% (w/w), about 30% (w/w), about 35% (w/w), about 40% (w/w), about 45% (w/w), about 50% (w/w), about 55% (w/w), about 60% (w/w), about 65% (w/w), about 70% (w/w), about 75% (w/w) or more of one or more of the disclosed SCP products.

Starting with wet or dry microbial biomass produced as described herein, in certain embodiments, protein may be concentrated using a process comprising one or more of the following steps: liquid-solid extraction, removal and recovery of a solvent from the liquid extract, removal and recovery of a solvent from the solid (e.g., the protein concentrate), and drying and grinding of the solid (e.g., the protein concentrate).

A solid-liquid extraction may be performed batchwise or continuously. The solid-liquid extraction may be performed using one or more of: horizontal belt extractors; basket extractors; stationary extractors; and/or rotary cell extractors.

A non-polar solvent may be utilized in a solvent extraction step. A non-polar solvent may be utilized in combination with an alcohol solvent. A non-polar solvent may be utilized in combination with an aqueous alcohol solution. A non-polar solvent can be utilized to extract neutral lipids from an extract produced using alcohol and/or an aqueous alcohol solution. The non-polar solvent may be utilized that has a boiling point range (i.e., distillation range) of 65° C. to 70° C. A non-polar solvent may be utilized that consists primarily of six-carbon alkanes. In certain aspects, hexane is utilized as a non-polar solvent. The hexane utilized as a non-polar solvent may comply with the strict quality specifications required for the extraction of edible oils from soybean and other plant-based sources, including but not limited to: boiling (distillation) range, maximum non-volatile residue, flash point, maximum sulfur, maximum cyclic hydrocarbons, color and specific gravity. In certain aspects, “supercritical extraction” using liquid carbon dioxide under high pressure is utilized for solvent extraction.

The cell mass, i.e., microbial biomass produced as described herein may be kept in liquid suspension when subjected to solvent extraction or if dried, may be fed as a loose power with open, porous structure into a solvent extraction process. The rate of extraction can be increased by applying one or more of agitation and/or increasing the temperature. Higher temperature can result in higher solubility of the extractable material (e.g., lipid), and/or higher diffusion coefficients.

Water-free (absolute) low aliphatic alcohols, such as ethanol or isopropanol, are suitable solvents for lipids at high temperature, but the solubility of oils in these solvents decreases drastically as the temperature is lowered. In certain embodiments, lipid extraction takes place at high temperature one or more alcohol, including but not limited to ethanol, isopropanol, and/or methanol. In certain such embodiments, the lipid extract is cooled, and lipid saturation occurs. In certain such embodiments, the excess lipid separates as a distinct phase, which can be recovered by a solid-liquid separation process, such as, but not limited to, centrifugation. In certain such embodiments, the solvent, i.e., alcohol(s), is reheated and sent back for solvent extraction.

When a concentration gradient is used to transfer the extractable substance out of a solid, keeping the gradient high can facilitate the extraction process. In certain embodiments, the principle of counter-current multistage extraction is utilized to exploit this effect. In certain embodiments, the solvent extraction process is divided into a number of contact stages. In certain embodiments, each stage comprises the mixing of solid, e.g., microbial biomass and/or protein concentrate, and the solvent phases, and the separation of the two streams after extraction is achieved. In certain embodiments, in going from one stage to the next, the solids, e.g., microbial biomass and/or protein concentrate, and the solvent flow in opposite directions. Thus, microbial biomass and/or protein concentrate with the lowest extractable content (e.g., lipids) are contacted with the leanest solvent, resulting in higher extractable yield (e.g., lipid yield) and high driving force throughout the extractor.

The cell culture may be harvested in a logarithmic phase and/or in an arithmetic phase and/or in a stationary phase. Extraction can be performed using batch, semi-continuous and/or continuous solvent extractors.

In batch processes, a certain quantity of microbial biomass and/or biological material is contacted with a certain volume of fresh solvent. In certain embodiments, the extract is drained off, distilled and the solvent is recirculated through the extractor until the residual extractable content (e.g., lipid content) in the batch of microbial biomass and/or biological material is reduced to a targeted level.

A semi-continuous solvent extraction system may utilize that consists of several batch extractors connected in series. In certain such embodiments, the solvent and/or extract flows from one extractor to the next one in the series. In certain non-limiting embodiments, a French Stationary Basket Extractor is utilized.

A continuous solvent extraction process may be utilized in which microbial biomass and/or biological material and/or protein concentrate and solvent are fed continuously into an extractor.

A protein product (e.g., one or more of single cell protein, cell lysate, protein concentrate, protein isolate, protein extract, protein hydrolysate, free amino acids, peptides, oligopeptides, or combinations thereof), is derived from and/or includes biomass and/or protein isolate, protein extract, protein hydrolysate, free amino acids, peptides, and/or oligopeptides derived from one or more engineered C. necator strains described herein and may be produced by any method described herein. The disclosed protein products provide the additional benefit of including desirable digestive enzymes, which means the products can be prepared without separate or additional fortification with digestive enzymes.

### VIII. Examples of Embodiments

The following non-limiting list of embodiments provides examples of particular implementations of the present disclosure. Those skilled in the art will understand that additional and alternative embodiments may also be disclosed here.
Embodiment 1. A strain of Cupriavidus necator, comprising a transgene encoding a heterologous digestive enzyme selected from a protease, a carbohydrase, a lipase, and a phytase.
Embodiment 2. The strain of C. necator of embodiment 1, wherein the digestive enzyme is a protease.
Embodiment 3. The strain of C. necator of embodiment 1, wherein the digestive enzyme is a carbohydrase.
Embodiment 4. The strain of C. necator of embodiment 1, wherein the digestive enzyme is a lipase.
Embodiment 5. The strain of C. necator of embodiment 1, wherein the digestive enzyme is a phytase.
Embodiment 6. The strain of C. necator of any one of embodiments 1-5, further comprising a second transgene encoding a second heterologous digestive enzyme selected from a protease, a carbohydrase, a lipase, and a phytase.
Embodiment 7. The strain of C. necator of embodiment 6, further comprising a third transgene encoding a third heterologous digestive enzyme selected from a protease, a carbohydrase, a lipase, and a phytase.
Embodiment 8. A strain of C. necator that expresses a one or more heterologous digestive enzymes selected from a protease, a carbohydrase, a lipase, a phytase, and any combination thereof.
Embodiment 9. The strain of C. necator of embodiment 8, wherein the strain of C. necator expresses at least two heterologous digestive enzymes selected from a protease, a carbohydrase, a lipase, and a phytase.
Embodiment 10. The strain of C. necator of embodiment 8, wherein the strain of C. necator expresses at least three heterologous digestive enzymes selected from a protease, a carbohydrase, a lipase, and a phytase.
Embodiment 11. The strain of C. necator of any one of embodiments 1-10, wherein the protease is a savinase or a keratinase.
Embodiment 12. The strain of C. necator of any one of embodiments 1-11, wherein the carbohydrase is a cellulase, a β-glucanase, a xylanase, an amylase, or a galactosidase.
Embodiment 13. The strain of C. necator of any one of embodiments 1-12, wherein the heterologous digestive enzyme is from a bacteria, a fungi, a cyanobacteria, or a eukaryote.
Embodiment 14. The strain of C. necator of embodiment 13, wherein the bacteria is a Bacillus species (sp).
Embodiment 15. The strain of C. necator of embodiment 14, wherein the Bacillus sp. is selected from Bacillus subtilis, Bacillus licheniformis, Bacillus amyloliquefaciens, and Bacillus lentus.
Embodiment 16. The strain of C. necator of embodiment 13, wherein the eukaryote is a yeast.
Embodiment 17. The strain of C. necator of any one of embodiments 1-16, wherein the transgene(s) is/are codon optimized for expression in Cupriavidus necator.
Embodiment 18. The strain of C. necator of any one of embodiments 1-17, wherein the strain of C. necator is modified, optionally by adaptive laboratory evolution or genetic modification.
Embodiment 19. The strain of C. necator of any one of embodiments 1-18, wherein the strain of C. necator grows autotrophically at up to 40° C.
Embodiment 20. The strain of C. necator of embodiments 18 or 19, wherein the strain of C. necator has a H₂:CO₂ uptake ratio that is lower than the H₂:CO₂ uptake ratio of a wild type or naturally occurring strain of C. necator cultured under corresponding temperatures and conditions.
Embodiment 21. The strain of C. necator of embodiment 20, wherein the strain of C. necator comprises a full or partial deletion of a gene or locus encoding a membrane-bound hydrogenase.
Embodiment 22. The strain of C. necator of embodiment 21, wherein the membrane-bound hydrogenase is hoxKGXZ.
Embodiment 23. The strain of C. necator of any one of embodiments 1-22, wherein the strain of C. necator produces about 40%, about 50%, about 60%, about 70%, about 80%, about 90%, or about 95% by weight of protein.
Embodiment 24. The strain of C. necator of any one of embodiments 1-23, wherein the strain of C. necator is deposited at DSM34774.
Embodiment 25. A method of preparing a single-cell protein product, comprising: (a) culturing the strain of C. necator of any one of embodiments 1-24, thereby creating biomass comprising protein and a digestive enzymes, (b) isolating the C. necator or the biomass, and (c) preparing a single-cell protein product from the C. necator or the biomass.
Embodiment 26. A method of producing a digestive enzyme, comprising culturing the strain of C. necator of any one of embodiments 1-24 such that the digestive enzyme is produced.
Embodiment 27. The method of embodiments 25 or 26, wherein culturing comprising growing the strain of C. necator in a fermentation tank.
Embodiment 28. The method of embodiment 27, wherein the fermentation tank is a gas fermentation tank.
Embodiment 29. The method of any one of embodiment 25-28, wherein culturing comprising growing the strain of C. necator in autotrophic conditions.
Embodiment 30. The method of embodiment 29, wherein the autotrophic conditions comprise providing carbon monoxide (CO) or carbon dioxide (CO₂) as a carbon source.
Embodiment 31. The method of any one of embodiments 25-30, wherein culturing comprising growing the strain of C. necator at a temperature above 30° C. and up to 40° C.
Embodiment 32. The method of any one of embodiments 26-31 further comprising isolating the digestive enzyme.
Embodiment 33. The method of any one of embodiments 26-31 further comprising isolating the strain of C. necator.
Embodiment 34. A single-cell protein (SCP) composition, comprising the strain of C. necator of any one of embodiments 1-24 or biomass created therefrom.
Embodiment 35. The SCP composition of embodiment 34, wherein the composition comprises at least about 40%, about 50%, about 60%, about 70%, about 80%, about 90%, or about 95% by weight of protein.
Embodiment 36. The SCP composition of embodiments 34 or 35, wherein the composition comprises at least about 40%, about 50%, about 60%, about 70%, about 80%, about 90%, or about 95% by weight of a digestive enzyme component.
Embodiment 37. The SCP composition of embodiment 36, wherein the digestive enzyme component comprises a protease, a carbohydrase, a lipase, a phytase, and any combination thereof.
Embodiment 38. The SCP composition of any one of embodiments 34-37, wherein the composition is produced by culturing the strain of C. necator of any one of embodiments 1-24, thereby creating biomass comprising protein and digestive enzyme, and isolating the C. necator or the biomass.
Embodiment 39. An edible product, comprising (a) the strain of C. necator of any one of embodiments 1-24 or biomass created therefrom, or (b) the SCP composition of any one of embodiments 34-38.
Embodiment 40. The edible product of embodiment 39, wherein the edible product is an animal feed.
Embodiment 41. The edible product of embodiment 40, wherein the animal feed is selected from a fish feed, a livestock or ruminant feed, a swine feed, goat feed, llama feed, turkey feed, a poultry feed, a rodent feed, a dog feed, and a cat feed.
Embodiment 42. The edible product of embodiment 39, wherein the edible product is a human food.
Embodiment 43. The edible product of embodiment 42, wherein the human food is selected from a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, and a cereal or cereal product.
Embodiment 44. A method of preparing an edible product, comprising mixing (a) the strain of C. necator of any one of embodiments 1-24 or biomass created therefrom, or (b) the SCP composition of any one of embodiments 34-38 with one or more edible ingredients.
Embodiment 45. A nutritive or consumable composition comprising a strain of C. necator described herein.
Embodiment 46. The composition according to embodiment 45, wherein the composition is or comprises a yogurt, a smoothie, a bread product, a pasta product, a nutritional bar, a chip or cracker, a plant-based meat substitute, a cheese, a plant-based cheese, a powdered nutritional supplement, a dairy product, a dairy replacement product, a meat product, a bakery product, a confection, a protein bar, a protein powder, a sport and/or energy drink, a protein shake and/or smoothie, noodles, instant noodles, a soup, an instant soup, a microwaveable food, a canned food, a freeze-dried food, a soft drink, a fruit juice drink, a vegetable drink, an infant formula, a toddler formula, a non-dairy milk, a coffee drink, a tea drink, a nutritional beverage, a powdered beverage, a nutritional supplement, a concentrated beverage, an alcoholic beverage, a cake mix, a rice cake, a flour product, chewing gum, gummies, chocolate, caramel, a cookie, chips, pretzels, crackers, biscuits, cakes, pies, a sauce, a processed seasoning, a flavor seasoning, a cooking mix, a curry, a stew, a dressing, an oils/fat, a butter, a margarine, a mayonnaise and other condiments, a lactic acid bacteria drink, an ice cream, a cream processed fish product, a processed livestock product, an agricultural canned product, a jam or marmalade, a pickled product, or a cereal or cereal product.
Embodiment 47. The composition according to any of embodiments 45 to 46, wherein the composition is incorporated into one or more articles, converted into one or more second products, end-user products, consumer products, or any combination thereof.
Embodiment 48. The composition according to any of embodiments 45 to 47, wherein the composition is incorporated into pet food or animal feed.
Embodiment 49: A recombinant C1-fixing microorganism comprising a transgene encoding a heterologous digestive enzyme selected from a protease, a carbohydrase, a lipase, and a phytase.
Embodiment 50: A recombinant C1-fixing microorganism that expresses a one of more heterologous digestive enzymes selected from a protease, a carbohydrase, a lipase, a phytase, and any combination thereof.
Embodiment 51: The recombinant C1-fixing microorganism of embodiments 49 or 50, wherein the microorganism is selected from the group consisting of Cupriavidus necator and Ralstonia eutropha.
Embodiment 52: The recombinant C1-fixing microorganism of any one of embodiments 48-51, wherein the microorganism does not natively produce polyhydroxyalkanoates (PHAs).
Embodiment 53: The microorganism of any one of embodiments 49-52, wherein the microorganism expresses at least two heterologous digestive enzymes selected from a protease, a carbohydrase, a lipase, and a phytase.
Embodiment 54: The strain of C. necator of embodiment 538, wherein the microorganism expresses at least three heterologous digestive enzymes selected from a protease, a carbohydrase, a lipase, and a phytase.
Embodiment 55: The microorganism of any one of embodiments 49-54, wherein the protease is a savinase or a keratinase.
Embodiment 56: The microorganism of any one of embodiments 49-55, wherein the carbohydrase is a cellulase, a β-glucanase, a xylanase, an amylase, or a galactosidase.
Embodiment 57: The microorganism of any one of embodiments 49-56, wherein the heterologous digestive enzyme is from a bacteria, a fungi, a cyanobacteria, or a eukaryote.
Embodiment 58: The microorganism of embodiment 57, wherein the bacteria is a Bacillus species (sp).
Embodiment 59: The microorganism of embodiment 58, wherein the Bacillus sp. is selected from Bacillus subtilis, Bacillus licheniformis, Bacillus amyloliquefaciens, and Bacillus lentus.
Embodiment 60: The microorganism of embodiment 57, wherein the eukaryote is a yeast.
Embodiment 61: The microorganism of any one of embodiments 49-60, wherein the transgene is codon optimized for the microorganism.
Embodiment 62: The microorganism of any one of embodiments 49-61, wherein the microorganism is modified, optionally by adaptive laboratory evolution or genetic modification.
Embodiment 63: The microorganism of any one of embodiments 49-62, wherein the microorganism grows autotrophically at up to 40° C.
Embodiment 64: The microorganism of embodiment 62 or 63, wherein the microorganism has a H₂:CO₂ uptake ratio that is lower than the H₂:CO₂ uptake ratio of a wild type or naturally occurring strain of C. necator cultured under corresponding temperatures and conditions.
Embodiment 65: The microorganism of embodiment 64, wherein the microorganism comprises a full or partial deletion of a gene or locus encoding a membrane-bound hydrogenase.
Embodiment 66: The microorganism of embodiment 65, wherein the membrane-bound hydrogenase is hoxKGXZ.
Embodiment 67: The microorganism of any one of embodiments 49-66, wherein the microorganism produces about 40%, about 50%, about 60%, about 70%, about 80%, about 90%, or about 95% by weight of protein.
Embodiment 68: A method of culturing the microorganism according to any one of embodiments 49-66.
Embodiment 69: A product comprising the microorganism according to any one of embodiments 49-66 or a biological component (e.g., protein, etc.) derived from the microorganism.

### IX. EXAMPLES

The following examples are given to illustrate the present disclosure. It should be understood, however, that the disclosure is not to be limited to the specific conditions or details described in these examples.

### Example 1—ALE Strain of Heat Resistant C. necator Bacteria

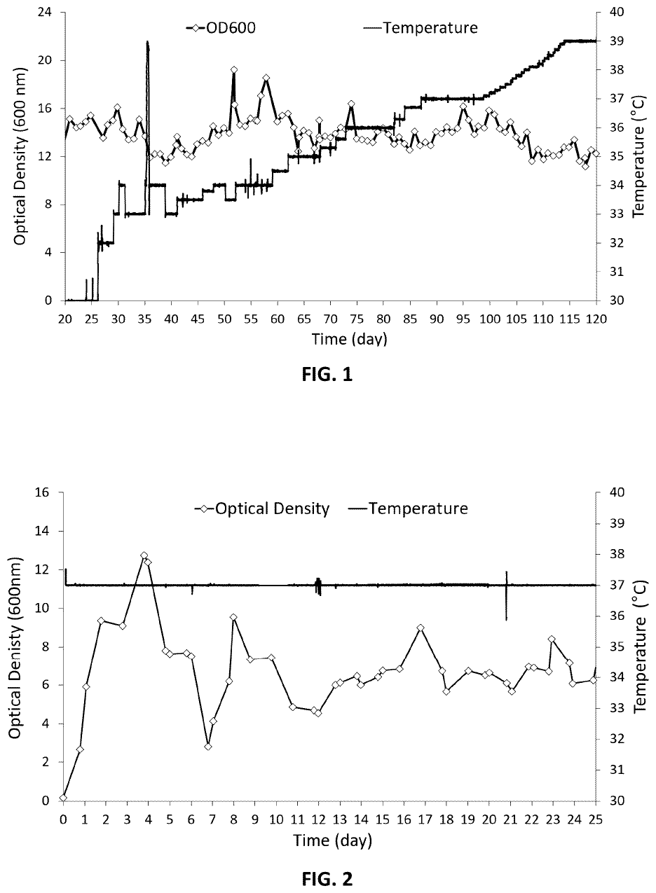

A continuous-flow stirred tank reactor (CSTR) was inoculated using a base strain of C. necator (H16 PHB-4) and grown continuously on minimal media and H₂/O₂/CO₂ inputs. The base strain, DSM 541, is a naturally occurring C. necator mutant that is unable to produce or express polyhydroxybutyrate (PHB). Once the culture was established and growing at steady-state, the reactor temperature was increased step-wise and held to allow the organism to adapt. These step changes were continued over ˜100 days and brought the reactor temperature from 30 to 39° C. (FIG. 1). Glycerol stocks were created and Whole Genome Sequencing conducted at various temperatures along the way to preserve progress and determine what beneficial mutations arose. Later, a separate CSTR was inoculated at 37° C. from the glycerol stock harvested at 37° C. during the evolution. This adapted strain started successfully, whereas other CSTRs using the base strain would only start at 30 and not 37° C., thus confirming the creation of a distinct phenotype.

As demonstrated in Table 1, the base strain was capable of growth on rich media during plate and inoculum growth at 30 to 37° C., but fails to grow autotrophically (minimal media, H₂/O₂/CO₂ feed) at 37° C. and above. However, the temperature evolved strain DSM 34774 surprisingly demonstrated growth under autotrophic conditions at 37° C. (Table 1, FIG. 2). Further, DSM 34774 was capable of growth on CO₂/H₂/O₂ gas mixtures at elevated temperatures of up to 39° C.

| Run # | 1 | 2 | 3 | 4 | 5 |
|---|---|---|---|---|---|
| Strain | DSM 541 | DSM 541 | DSM 541 | DSM 541 | DSM 34774 |
| Plate growth [° C.] | 30 | 30 | 37 | 37 | 37 |
| Inoculum growth [° C.] | 30 | 30 | 37 | 37 | 37 |
| Reactor startup [° C.] | 30 | 37 | 37 | 30 | 37 |
| Successful startup? | Yes | No | No | Yes | Yes |

The evolved strain DSM 34744 was equally capable of sufficient culture and growth at lower temperatures ranging from 30-37° C.

### Example 2—Expression of Protease Enzymes in Cupriavidus necator

Heat adapted strains of C. necator were transformed to express a savinase derived from Bacillus amyloliquefaciens or a savinase derived from Bacillus lentus. Genes encoding these proteases and linked HiBiT tags were expressed via rhamnose inducible promoter.

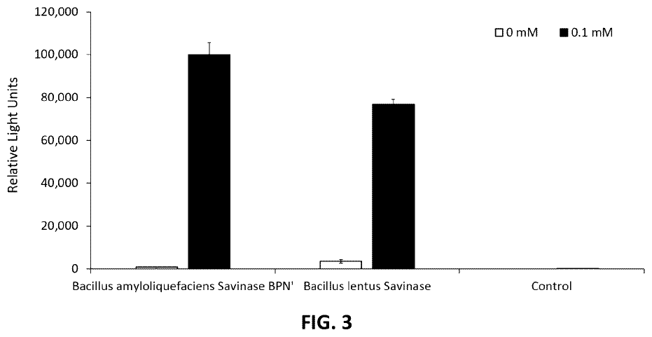

Protease expression in total cell lysate of synthetic constructs with HiBiT tags detected using was analyzed via a HiBiT Lytic Detection System. As demonstrated in FIG. 3, protease expression resulted in significant expression detection upon addition of 0.1 mM rhamnose indicating significant protease enzyme overexpression in heat adapted strains of C. necator.

### Example 3—Expression and Activity of Phytase Enzymes in Cupriavidus necator

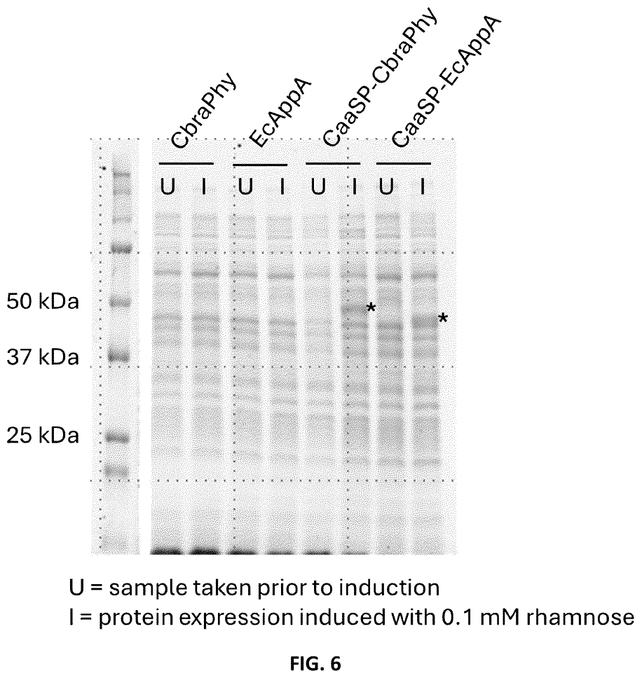

As demonstrated in FIG. 5, heat adapted strains of C. necator were transformed to express a phytase derived from Escherichia coli (EcAppA) or a phytase derived from Citrobacter braakii (CbraPhy). The phytase amino acid sequence was either modified or unmodified with the periplasmic targeting sequence CaaSP. Genes were codon optimized for C. necator and expressed via a rhamnose inducible promoter. Phytase expression was evaluated with a SDS-PAGE gel comparing samples before induction and 2 days after the addition of 0.1 mM rhamnose. Bands were observed for phytases with the CaaSP sequence. Phytase activity was evaluated in clarified lysate by measuring the release of phosphate from 5 mM phytic acid over 2 hr incubation at 50° C. Phytase activity was normalized to total protein. Phosphate was measured via malachite green assay kit (Sigma-Aldrich, MAK307). Total protein was measured via bicinchoninic acid assay. Activity above a negative control strain was observed for phytases CbrBraPhy, EcAppA, and CaaSP-CbraPhy. As depicted in FIG. 6, expression of phytase enzymes in Cupravidus necator. Overproduction of two phytases, CaaSP-CbraPhy and CaaSP-EcAppA, in C. necator was observed in an SDS-PAGE gel stained with Coomassie. An extra protein band was observed at the correct sizes (48.8 kDa and 47.4 kDa, respectively) in the induced lane relative to the uninduced lane.

It should be appreciated that all combinations of the disclosed concepts are provided as being part of the inventive subject matter disclosed herein and may be employed in any combination to achieve the benefits described herein.

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

1. A strain of Cupriavidus necator, comprising a transgene encoding a heterologous digestive enzyme selected from a protease, a carbohydrase, a lipase, and a phytase.

2. The strain of C. necator of claim 1, wherein the digestive enzyme is a protease.

3. The strain of C. necator of claim 1, wherein the digestive enzyme is a carbohydrase.

4. The strain of C. necator of claim 1, wherein the digestive enzyme is a lipase.

5. The strain of C. necator of claim 1, wherein the digestive enzyme is a phytase.

6. The strain of C. necator of claim 1, further comprising a second transgene encoding a second heterologous digestive enzyme selected from a protease, a carbohydrase, a lipase, and a phytase.

7. The strain of C. necator of claim 6, further comprising a third transgene encoding a third heterologous digestive enzyme selected from a protease, a carbohydrase, a lipase, and a phytase.

8. A strain of C. necator that expresses a one or more heterologous digestive enzymes selected from a protease, a carbohydrase, a lipase, a phytase, and any combination thereof.

9. The strain of C. necator of claim 8, wherein the strain of C. necator expresses at least two heterologous digestive enzymes selected from a protease, a carbohydrase, a lipase, and a phytase.

10. The strain of C. necator of claim 8, wherein the strain of C. necator expresses at least three heterologous digestive enzymes selected from a protease, a carbohydrase, a lipase, and a phytase.

11. The strain of C. necator of claim 1, wherein the protease is a savinase or a keratinase.

12. The strain of C. necator of claim 1, wherein the carbohydrase is a cellulase, a β-glucanase, a xylanase, an amylase, or a galactosidase.

13. The strain of C. necator of claim 1, wherein the heterologous digestive enzyme is from a bacteria, a fungi, a cyanobacteria, or a eukaryote.

14. The strain of C. necator of claim 13, wherein the bacteria is a Bacillus species (sp).

15. The strain of C. necator of claim 14, wherein the Bacillus sp. is selected from Bacillus subtilis, Bacillus licheniformis, Bacillus amyloliquefaciens, and Bacillus lentus.

16. The strain of C. necator of claim 13, wherein the eukaryote is a yeast.

17. The strain of C. necator of claim 1, wherein the transgene(s) is/are codon optimized for expression in Cupriavidus necator.

18. The strain of C. necator of claim 1, wherein the strain of C. necator is modified, optionally by adaptive laboratory evolution or genetic modification.

19. The strain of C. necator of claim 1, wherein the strain of C. necator grows autotrophically at up to 40° C.

20. The strain of C. necator of claim 18, wherein the strain of C. necator has a H₂:CO₂ uptake ratio that is lower than the H₂:CO₂ uptake ratio of a wild type or naturally occurring strain of C. necator cultured under corresponding temperatures and conditions.
